(19) United States
(12) Patent Application Publication
(10) Pub. No.: US 2023/0128114 A1
(43) Pub. Date: Apr. 27, 2023

(54) METHODS AND COMPOSITIONS FOR TREATING OR PREVENTING AN INFLAMMATORY CONDITION

(71) Applicant: Incannex Healthcare Limited, Norwest, New South Wales (AU)

(72) Inventors: Joel Bradley LATHAM, North Kellyville (AU); Sudhanshu AGARWAL, Toorak (AU); Mark Robert BLEACKLEY, South Morang (AU)

(21) Appl. No.: 17/907,322

(22) PCT Filed: Mar. 15, 2021
(86) PCT No.: PCT/AU2021/050226

§ 371 Date: Sep. 26, 2022

(30) Foreign Application Priority Data
Apr. 2, 2020 (AU) 2020901030
Jul. 14, 2020 (AU) 2020902432
Nov. 2, 2020 (AU) 2020903985
Nov. 18, 2020 (AU) 2020904264
Feb. 3, 2021 (AU) 2021900241
Feb. 10, 2021 (AU) 2021900324

(51) Int. Cl. A61K 31/05; A61K 31/4706; A61K 9/00; A61P 37/02

(52) U.S. Cl. CPC A61K 31/05; A61K 31/4706; A61K 9/0053; A61P 37/02

(57) ABSTRACT

Compositions including cannabidiol (CBD) and hydroxychloroquine (HCQ) or pharmaceutically acceptable salts thereof, and their use in methods for the treatment of an inflammatory condition, or for modulating an immune response. The methods and compositions are useful for the treatment or prevention of an inflammatory condition, including but not limited to, inflammatory bowel disease, arthritis, and inflammatory respiratory conditions, such as chronic obstructive pulmonary disease (COPD), asthma, bronchitis, cystic fibrosis (CF) and acute respiratory distress syndrome (ARDS).

### RELATED APPLICATIONS

This application claims priority from Australian Provisional Patent Application Nos. 2020901030, 2020902432, 2020903985, 2020904264, 2021900241 and 2021900324, filed on 2 Apr. 2020, 14 Jul. 2020, 2 Nov. 2020, 18 Nov. 2020, 3 Feb. 2021 and 10 Feb. 2021, respectively, the entire contents of which are hereby incorporated by reference.

### FIELD

The present disclosure relates generally to methods and compositions useful for modulating an inflammatory response. In an embodiment, the present disclosure relates to methods and compositions useful for the treatment or prevention of an inflammatory condition, including but not limited to, inflammatory bowel disease, arthritis, and inflammatory respiratory conditions, such as chronic obstructive pulmonary disease (COPD), asthma, bronchitis, cystic fibrosis (CF) and acute respiratory distress syndrome (ARDS).

### BACKGROUND

Inflammatory conditions are characterised by inflammation, the complex biological response to a noxious stimulus such as damage, auto-immunity, or an infection by a microbial pathogen and/or virus. The clinical features of an inflammatory condition are likely to depend on the noxious stimulus (or stimuli), but are typically associated with heat, pain, redness and swelling of the affected organ or tissue.

Inflammatory conditions are commonly associated with the upregulation of pro-inflammatory cytokines, such as interleukin-1 beta (IL-1β), interleukin-6 (IL-6), tumour-necrosis factor alpha (TNF-α), interleukin-1 alpha (IL-1α), interleukin-12 (IL-12(p70)), interferon gamma (IFN-γ) and/or macrophage inflammatory protein (MIP-1α), which modulate a range of signaling pathways to promote inflammatory reactions. Inflammatory conditions associated with the upregulation of pro-inflammatory cytokines include arthritis, inflammatory bowel disease, pain, gout, fibromyalgia, endometriosis, alcoholic liver disease, psoriasis/dermatitis, lupus and inflammatory respiratory conditions such as acute respiratory distress syndrome (ARDS), chronic obstructive pulmonary disease (COPD), asthma, bronchitis, bronchiectasis, sarcoidosis and pulmonary inflammation.

Inflammatory respiratory conditions are among the most prevalent diseases globally. Although different inflammatory respiratory conditions express different inflammatory responses, there are some shared pathological features. For example, the innate neutrophilic inflammation shared by COPD and ARDS is also observed in some patients with severe asthma.

ARDS is an inflammatory condition affecting pulmonary tissue, which is most commonly caused by sepsis. ARDS is characterised by poor oxygenation, pulmonary infiltrates, and acuity of onset (Diamond et al. 2020, Acute Respiratory Distress Syndrome (ARDS), in: StatPearls; Treasure Island (FL): StatPearls Publishing; 2020). ARDS typically begins within a week of the inciting event with a clinical presentation including capillary endothelial injury and diffuse alveolar damage, bilateral lung infiltrates and severe progressive hypoxemia in the absence of any evidence of cardiogenic pulmonary edema. Once ARDS develops, patients usually have varying degrees of pulmonary artery vasoconstriction and, subsequently, may develop pulmonary hypertension.

ARDS has many risk factors. Besides pulmonary infection or aspiration, extra-pulmonary causes include sepsis, trauma, transfusion, near drowning, drug overdose, fat embolism, inhalation injury, and pancreatitis. These extra-thoracic illnesses and/or injuries trigger an inflammatory cascade culminating in pulmonary injury.

Sepsis-associated ARDS is a complication arising from severe sepsis, being a systemic response to an infection of the blood. Any viable microbe, such as bacteria, fungi and viruses, can be the source of the infection. Sepsis is relatively common, occurring in approximately 750,000 adults annually in the United States, of which approximately 6% of patients develop ARDS. The mortality rate of patients diagnosed with ARDS is high, with an acute mortality rate exceeding 30% despite optimal critical care.

The main pathophysiologic mechanism underlying ARDS is unrestrained and perpetual inflammation, resulting in edema. Respiratory failure leaves to multiple organ removal from the alveolar space, resulting in the accumulation of protein-rich fluid inside the alveoli. Edema produces diffuse alveolar damage, with the release of pro-inflammatory cytokines, including TNF-α, IL-1β, and IL-6. Neutrophils are recruited to the lungs by cytokines, become activated and release toxic mediators, such as reactive oxygen species and proteases. Extensive free radical production overwhelms endogenous anti-oxidants and causes oxidative cell damage. The initial phase of ARDS is the exudative phase, which is characterised by fluid accumulation in the pulmonary tissue, followed by a proliferation phase, which is characterised by resolution of pulmonary edema, proliferation of type II alveolar cells, fibroblasts, and myofibroblasts, and new matrix deposition. The proliferation phase starts within 72 hours after disease onset and lasts for more than 7 days. Patients who develop pulmonary fibrosis exhibit deterioration of pulmonary compliance, progressive hypoxia, and ventilator dependence, with mortality rate of nearly 60% (Pierrakos et al., 2012, Journal of Clinical Medicine Research, 4: 7-16).

Current standard of care for the treatment of sepsis-associated ARDS is limited to supporting therapies, such as mechanical ventilation and oxygen supplementation, which is typically delivered concurrently with treatments for the underlying infection causing the severe sepsis, if any such treatments are available. However, despite decades of efforts to improve the prognosis of ARDS, no curative treatments are currently available. Previous attempts to develop treatments for ARDS have aimed to reduce the acute pulmonary inflammatory response, reversal of edema, and limiting damage to the lung using, for example, glucocorticoids (Steinberg et al., 2006, The New England Journal of Medicine, 354(16): 1671-1684), surfactant replacement therapy, neutrophil elastase inhibition, anticoagulation drugs, non-steroidal anti-inflammatory drugs and statins (reviewed in, e.g., Boyle et al., 2013, BMC Medicine, 11:166). Unfortunately, however, no pharmacological interventions have been proven to be effective for the treatment of ARDS.

Therefore, there remains an urgent need for the development of pharmacological approaches for the treatment or prevention of inflammatory conditions, including inflammatory bowel disease, arthritis and inflammatory respiratory conditions, such as COPD, asthma, bronchitis, CF and ARDS.

### SUMMARY

In an aspect of the present disclosure, there is provided a method for the treatment or prevention of an inflammatory condition, the method comprising administering to a subject in need thereof an effective amount of cannabidiol (CBD) or a pharmaceutically acceptable salt thereof, and an effective amount of hydroxychloroquine or a pharmaceutically acceptable salt thereof.

In another aspect of the present disclosure, there is provided a use of CBD or a pharmaceutically acceptable salt thereof, and hydroxychloroquine or a pharmaceutically acceptable salt thereof, in the manufacture of medicament for the treatment or prevention of an inflammatory condition.

In another aspect of the present disclosure, there is provided a composition comprising CBD or a pharmaceutically acceptable salt thereof, and hydroxychloroquine or a pharmaceutically acceptable salt thereof.

In another aspect of the present disclosure, there is provided a composition comprising CBD or a pharmaceutically acceptable salt thereof, and hydroxychloroquine or a pharmaceutically acceptable salt thereof, for use in the treatment or prevention of an inflammatory condition.

In another aspect of the present disclosure, there is provided a kit comprising CBD or a pharmaceutically acceptable salt thereof, and hydroxychloroquine or a pharmaceutically acceptable salt thereof, for use in the treatment or prevention of an inflammatory condition.

In another aspect of the present disclosure, there is provided a method of modulating an immune response comprising administering to a subject in need thereof CBD or a pharmaceutically acceptable salt thereof, and hydroxychloroquine or a pharmaceutically acceptable salt thereof.

### BRIEF DESCRIPTION OF THE DRAWINGS

FIG. 1 shows that CBD or hydroxychloroquine can modulate key immune markers associated with sepsis-associated ARDS following intraperitoneal (IP) administration. (A) a graphical representation of mouse IL-1β (pg/mL; y-axis) following IP administration of CBD or hydroxychloroquine (x-axis); (B) a graphical representation of mouse IL-2 (pg/mL; y-axis) following IP administration of CBD or hydroxychloroquine (x-axis); (C) a graphical representation of mouse IL-10 (pg/mL; y-axis) following IP administration of CBD or hydroxychloroquine (x-axis); (D) a graphical representation of mouse IL-12(p70) (pg/mL; y-axis) following IP administration of CBD or hydroxychloroquine (x-axis); (E) a graphical representation of mouse IFN-γ (pg/mL; y-axis) following IP administration of CBD or hydroxychloroquine (x-axis); (F) a graphical representation of mouse TNF-α (pg/mL; y-axis) following IP administration of CBD or hydroxychloroquine (x-axis); (G) a graphical representation of D-dimer level (pg/mL; y-axis) following IP administration of CBD or hydroxychloroquine (x-axis); +p<0.05, vs. sham; *p<0.05, vs. vehicle for CBD (Group 2); #p<0.05, vs. vehicle for hydroxychloroquine (Group 10); one-way ANOVA and Dunnett's test.

FIG. 2 shows that CBD and/or hydroxychloroquine can modulate key immune markers associated with sepsis-associated ARDS following oral administration. (A) a graphical representation of mouse IL-1α (pg/mL; y-axis) following oral administration of CBD and/or hydroxychloroquine (x-axis); (B) a graphical representation of mouse IL-1β (pg/mL; y-axis) following oral administration of CBD and/or hydroxychloroquine (x-axis); (C) a graphical representation of mouse IL-2 (pg/mL; y-axis) following oral administration of CBD and/or hydroxychloroquine (x-axis); (D) a graphical representation of mouse IL-3 (pg/mL; y-axis) following oral administration of CBD and/or hydroxychloroquine (x-axis); (E) a graphical representation of mouse IL-4 (pg/mL; y-axis) following oral administration of CBD and/or hydroxychloroquine (x-axis); (F) a graphical representation of mouse IL-5 (pg/mL; y-axis) following oral administration of CBD and/or hydroxychloroquine (x-axis); (G) a graphical representation of mouse IL-6 (pg/mL; y-axis) following oral administration of CBD and/or hydroxychloroquine (x-axis); (H) a graphical representation of mouse IL-9 (pg/mL; y-axis) following oral administration of CBD and/or hydroxychloroquine (x-axis); (I) a graphical representation of mouse IL-10 (pg/mL; y-axis) following oral administration of CBD and/or hydroxychloroquine (x-axis); (J) a graphical representation of mouse IL-12(p40) (pg/mL; y-axis) following oral administration of CBD and/or hydroxychloroquine (x-axis); (K) a graphical representation of mouse IL-12(p70) (pg/mL; y-axis) following oral administration of CBD and/or hydroxychloroquine (x-axis); (L) a graphical representation of mouse IL-13 (pg/mL; y-axis) following oral administration of CBD and/or hydroxychloroquine (x-axis); (M) a graphical representation of mouse IL-17A (pg/mL; y-axis) following oral administration of CBD and/or hydroxychloroquine (x-axis); (N) a graphical representation of mouse Eotaxin (pg/mL; y-axis) following oral administration of CBD and/or hydroxychloroquine (x-axis); (O) a graphical representation of mouse G-CSF (pg/mL; y-axis) following oral administration of CBD and/or hydroxychloroquine (x-axis); (P) a graphical representation of mouse GM-CSF (pg/mL; y-axis) following oral administration of CBD and/or hydroxychloroquine (x-axis); (Q) a graphical representation of mouse IFN-γ (pg/mL; y-axis) following oral administration of CBD and/or hydroxychloroquine (x-axis); (R) a graphical representation of mouse keratinocyte chemoattractant (KC) (pg/mL; y-axis) following oral administration of CBD and/or hydroxychloroquine (x-axis); (S) a graphical representation of mouse MCP-1 (pg/mL; y-axis) following oral administration of CBD and/or hydroxychloroquine (x-axis); (T) a graphical representation of mouse MIP-1α (pg/mL; y-axis) following oral administration of CBD and/or hydroxychloroquine (x-axis); (U) a graphical representation of mouse MIP-1β (pg/mL; y-axis) following oral administration of CBD and/or hydroxychloroquine (x-axis); (V) a graphical representation of mouse RANTES (pg/mL; y-axis) following oral administration of CBD and/or hydroxychloroquine (x-axis); (W) a graphical representation of mouse TNF-α (pg/mL; y-axis) following oral administration of CBD and/or hydroxychloroquine (x-axis); p<0.05, vs. Vehicle control; one-way ANOVA and Dunnett's test.

FIG. 3 shows that CBD and hydroxychloroquine are effective for the treatment of mice with TNBS-induced colitis. (A) A photographic representation of normal colon tissue in sham-treated control mice; (B) A photographic representation of colon tissue from vehicle control mice, *indicates regions of inflammatory cell infiltration in submucosal edema; (C) A photographic representation of colon tissue from mice treated with CBD (1 mg/kg), *indicates regions of inflammatory cell infiltration in submucosal edema; (D) A photographic representation of colon tissue from mice treated with hydroxychloroquine (2.5 mg/kg), *indicates regions of mild abnormality, cyctic dilation and aberrant crypts; (E) A photographic representation of colon tissue from mice treated with CBD (1 mg/kg) and hydroxychloroquine (2.5 mg/kg), *indicates regions of minimal cell infiltration. All images shown at 100× magnification, and stained with hematoxylin and eosin (H&E).

FIG. 4 shows that CBD and hydroxychloroquine are effective for the treatment of mice with pulmonary inflammation. (A) A photographic representation of normal lung tissue in sham-treated control mice; (B) A photographic representation of lung tissue from vehicle control mice; (C) A photographic representation of lung tissue from mice treated with CBD (1 mg/kg); (D) A photographic representation of lung tissue from mice treated with hydroxychloroquine (2.5 mg/kg); (E) A photographic representation of lung tissue from mice treated with CBD (1 mg/kg) and hydroxychloroquine (2.5 mg/kg); (F) A photographic representation of lung tissue from mice treated with CBD (10 mg/kg); (G) A photographic representation of lung tissue from mice treated with hydroxychloroquine (25 mg/kg); and (G) A photographic representation of lung tissue from mice treated with CBD (10 mg/kg) and hydroxychloroquine (25 mg/kg). All images shown at 100× magnification, and stained with hematoxylin and eosin (H&E). Arrows indicate inflammatory cell infiltration.

FIG. 5 shows that CBD and hydroxychloroquine are effective for the treatment of rats with collagen-induced arthritis. (A) A photographic representation of normal hind paw ankle tissue in sham-treated control rats; (B) A photographic representation of hind paw ankle tissue from vehicle control rats; (C) A photographic representation of hind paw ankle tissue from rats treated with CBD (1 mg/kg); (D) A photographic representation of hind paw ankle tissue from rats treated with hydroxychloroquine (2.5 mg/kg); (E) A photographic representation of hind paw ankle tissue from rats treated with CBD (1 mg/kg) and hydroxychloroquine (2.5 mg/kg). All images shown at 50× magnification, and stained with hematoxylin and eosin (H&E).

### DETAILED DESCRIPTION

Unless defined otherwise, all technical and scientific terms used herein have the same meaning as commonly understood by those of ordinary skill in the art to which the disclosure belongs. Any materials and method similar or equivalent to those described herein can be used to practice the present invention.

Throughout this specification, unless the context requires otherwise, the word “comprise”, or variations such as “comprises” or “comprising”, will be understood to imply the inclusion of the stated element or integer or group of elements or integers but not the exclusion of any other element or integer or group of elements or integers.

The phrase “consisting of” means including, and limited to, whatever follows the phrase “consisting of”. Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a compound” includes a single compound, as well as two or more compounds; reference to “an agent” includes one agent, as well as two or more agents; and so forth.

The term “about” will be understood by persons skilled in the art and will vary to some extent depending on the context in which it is used. If there are uses of the term that are not clear to persons skilled in the art, given the context which it is used, “about” will mean up to plus or minus 10% of the particular term.

The present disclosure is predicated, at least in part, on the inventor's surprising finding that the administration of cannabidiol (CBD) and hydroxychloroquine, can synergise to provide potent anti-inflammatory activity, useful for the treatment of arthritis, inflammatory bowel disease and inflammatory respiratory conditions including acute respiratory distress syndrome (ARDS), cystic fibrosis (CF), asthma, bronchitis and chronic obstructive pulmonary disease (COPD). For example, the synergistic effect between CBD and hydroxychloroquine enables a reduction in neutrophil infiltration into the pulmonary tissue to both reduce the proliferation phase of ARDS, and promote the clearance of alveolar fluid. Moreover, the combination of CBD and hydroxychloroquine can reduce the formation and/or severity of colitis lesions and ameliorate symptoms of inflammatory bowel disease, including reduction in myeloperoxidase (MPO) levels in the colon, stool consistency score and reduction in macroscopic damage, thereby enabling the therapeutic use of this combination for patients with inflammatory bowel disease. Further, the combination of CBD and hydroxychloroquine has been shown to synergise to the extent that makes it possible to reduce the dose of hydroxychloroquine to a level that may minimise adverse effects associated with long-term use, while still eliciting a therapeutic effect.

Thus, in an aspect disclosed herein, there is provided a method for the treatment or prevention of an inflammatory condition, the method comprising administering to a subject in need thereof an effective amount of CBD or a pharmaceutically acceptable salt thereof, and an effective amount of hydroxychloroquine or a pharmaceutically acceptable salt thereof.

### Inflammatory Conditions

As used herein, the term “inflammatory condition” typically refers to a condition characterised by inflammation, or the complex biological response to a noxious stimulus such as damage, auto-immunity, or an infection by a microbial pathogen and/or virus. The clinical features of an inflammatory condition are likely to depend on the noxious stimulus (or stimuli), but is typically characterised by heat, pain, redness and swelling of the affected organ or tissue. The inflammatory condition may be acute or chronic.

In an embodiment, the inflammatory condition is selected from the group consisting of an inflammatory respiratory condition, inflammatory bowel disease and arthritis.

In an embodiment, the inflammatory condition is an inflammatory respiratory condition.

Inflammatory respiratory conditions would be known to persons skilled in the art, illustrative examples of which include ARDS, chronic obstructive pulmonary disease (COPD), asthma, bronchitis, bronchiectasis, sarcoidosis and cystic fibrosis (CF).

In an embodiment, the inflammatory respiratory condition is selected from the group consisting of chronic obstructive pulmonary disease (COPD), asthma, bronchitis, cystic fibrosis (CF) and acute respiratory distress syndrome (ARDS).

In an embodiment, the inflammatory respiratory condition is ARDS. In another embodiment, the inflammatory respiratory condition is sepsis-associated ARDS.

In an embodiment, the inflammatory condition is an inflammatory bowel disease.

The term “inflammatory bowel disease” refers to diseases or disorders that involve chronic inflammation of the digestive tract. Such diseases or disorders would be known to persons skilled in the art, illustrative examples of which include ulcerative colitis, and Crohn's disease.

In an embodiment, the inflammatory condition is arthritis.

The term “arthritis” typically refers to diseases that are characterised by inflammation of the joints that may also include the loss of cartilage. Such diseases would be known to persons skilled in the art, illustrative examples of which include rheumatoid arthritis, osteoarthritis, psoriatic arthritis and ankylosing spondylitis.

In an embodiment, the arthritis is rheumatoid arthritis.

In an embodiment, the inflammatory condition is associated with an increase or upregulation in the level of an inflammatory cytokine selected from the group consisting of IL-1β, IL-6, TNF-α, IL-1α, IL-12(p70), IFN-γ, CXCL-1, MCP-1 and MIP-1α, or combinations thereof.

In an embodiment, the inflammatory condition is associated with an increase or upregulation in the level of an inflammatory cytokine selected from the group consisting of IL-1β, IL-6 and TNF-α, or combinations thereof.

Inflammatory conditions associated with an increase or upregulation in the level of IL-1β, IL-6, TNF-α, IL-1α, IL-12(p70), IFN-γ, CXCL-1, MCP-1 and/or MIP-1α would be known to persons skilled in the art, illustrative examples of which include arthritis (as described by, e.g., Feldman et al., 1996, Annual Review of Immunology, 14: 397-440; McInnes et al., 2007, Nature Reviews Immunology, 7: 429-442; Tanaka et al., 2014, Cold Spring Harbor Perspectives in Biology, 6: a016292-a016295; Woo, 2002, Current Rheumatology Reports, 4: 452-457; Kapoor et al., 2011, Nature Reviews Rheumatology, 7: 33-42), inflammatory bowel disease (as described by, e.g., Neurath, 2014, Nature Reviews Immunology, 14: 329-342; Papadakis and Targan, 2000, The Annual Review of Medicine, 51: 289-298), pain (as described by, e.g., Zhang, 2007, International Anesthesiology Clinics, 45: 27-37), gout (as described by, e.g., Busso, 2010, Arthritis Research & Therapy, 12: 206), fibromyalgia (as described by, e.g., Rodriguez-Pintó et al., 2014, Immunology Letters, 161: 200-203), endometriosis (as described by, e.g., Wu and Ho, 2003, American Journal of Reproductive Immunology, 49: 285-296), chronic obstructive pulmonary disease (as described by, e.g., Chung, 2001, European Respiratory Journal, 18: 50s-59s), asthma (as described by, e.g., Rincon and Irvin, 2012, International Journal of Biological Sciences, 8: 1281-1290; Thomas, 2001, Immunology & Cell Biology, 79: 132-140), alcoholic liver disease (as described by, e.g., McClain et al., 1999, in Seminars in Liver Diseases, 205-220; Kawaratani et al., 2013, Mediators of Inflammation, 2013: 495156), psoriasis/dermatitis (as described by, e.g., Jensen, 2010, Current Opinions in Investigative Drug Discovery, 11: 1211-1220; Baliwag et al., 2015, Cytokine, 31: 781-789; Toshitani et al., 1993, Journal of Investigative Dermatology, 100: 299-304), and lupus (as described by, e.g., Davis et al., 2011, Journal of Interferon & Cytokine Research, 31: 781-789).

### Acute Respiratory Distress Syndrome (ARDS)

The terms “acute respiratory distress syndrome”, “adult respiratory distress syndrome”, or “ARDS” may be used interchangeably herein to refer to an inflammatory condition typically characterised by the disruption of the alveolar-capillary barrier, flooding of protein-rich edema fluid into the alveolar space, and cell recruitment due to immune system stimulation. ARDS can develop after direct lung injuries, e.g., pneumonia, aspiration, inhalation injury, near drowning, pulmonary contusion, reperfusion pulmonary edema and fat embolism. ARDS can also occur during the course of indirect lung injuries, e.g., sepsis, severe trauma, acute pancreatitis, cardiopulmonary bypass, massive transfusions and drug overdose.

“Sepsis-associated acute respiratory distress syndrome” or “sepsis-associated ARDS” typically refers to ARDS that is induced after lung infection or infection at extra-pulmonary sites. An aberrant host response to infection leads to disruption of the pulmonary alveolar-capillary barrier, resulting in lung injury characterised by hypoxemia, inflammation, and non-cardiogenic pulmonary edema.

In an embodiment, the ARDS is sepsis-associated ARDS.

The types of pathogens that can cause sepsis-associated ARDS would be known to persons skilled in the art, illustrative examples of which include bacteria, e.g., Streptococcus pneumoniae, Staphylococcus aureus, Pseudomonas aeruginosa, Acinetobacter baumannii; fungi, e.g., Pneumocystis jirovecii, Candida albicans, Candida tropicalis, Candida glabrata; and virus, e.g., cytomegalovirus, influenza, herpes simplex virus-1, respiratory syncytial virus, parainfluenza, human metapneumovirus, enterovirus and coronavirus.

In an embodiment, the sepsis-associated ARDS is caused by a bacterial, fungal or viral infection. In an embodiment, the bacterial infection is selected from the group consisting of Streptococcus pneumoniae, Staphylococcus aureus, Pseudomonas aeruginosa and Acinetobacter baumannii infection. In an embodiment, the viral infection is selected from the group consisting of cytomegalovirus, influenza, herpes simplex virus-1, respiratory syncytial virus, parainfluenza, human metapneumovirus, enterovirus and coronavirus infection. In an embodiment, the fungal infection is selected from the group consisting of Pneumocystis jirovecii, Candida albicans, Candida tropicalis and Candida glabrata infection.

ARDS may be classified using the Berlin Criteria as described by, for example, The ARDS Definition Task Force (2012, American Medical Association, 307(23): 2526-2234), which is based on the ration of arterial oxygen tension and fraction of inspired oxygen when measured at a minimum level of positive end-expiratory pressure (PEEP) of 5 cm H₂O. According to the Berlin Criteria, the severity of ARDS may be classified as mild, moderate or severe (Table 1).

TABLE 1
Classification of ARDS according to ratio of arterial oxygen
tension and fraction of inspired oxygen (PaO₂:FiO₂)

| Classification | PaO₂:FiO₂ |
|---|---|
| Mild | >200 mmHg and ≤300 mmHg¹ |
| Moderate | >100 mmHg and ≤200 mmHg² |
| Severe | ≤100 mmHg² |

¹As measured on ventilator settings that include positive end-expiratory pressure (PEEP) or continuous positive airway pressure (CPAP) ≥ 5 cm H₂O;
²as measured on ventilator settings that include PEEP ≥ 5 cm H₂O.

The classification of ARDS based on the Berlin Criteria may also be made in consideration of risk factors and period of exposure to risk factors (i.e., maximum period between exposure to risk factor and ARDS development is 7 days). Risk factors of ARDS include direct risk factors, i.e., pneumonia, aspiration of gastric contents, inhalation injury, pulmonary contusion, lung vasculitis and drowning, and indirect risk factors, i.e., non-pulmonary sepsis, multiple trauma, pancreatitis, non-cardiogenic shock, drug overdose and transfusion-associated acute lung injury (TRALI).

Imaging the lung pathobiology in ARDS patients and measurement of inflammatory markers or other biomarkers for ARDS may also be used in the classification of ARDS.

Suitable imaging methods would be known to persons skilled in the art, illustrative examples of which include computed tomography (CT), as described by, for example, Puybasset et al. (1998, American Journal of Respiratory and Critical Care Medicine, 158(5): 1644-1655). Similarly, suitable inflammatory markers and other biomarkers for ARDS would be known to persons skilled in the art, illustrative examples of which include the biomarkers reviewed by, for example, Blondonnet et al. (2016, Disease Markers, 2016: 35101373).

The present inventors have surprisingly found that CBD and hydroxychloroquine act synergistically to inhibit the production of inflammatory cytokines in response to a noxious stimulus. The present inventors have also surprisingly found that administration of CBD and hydroxychloroquine can treat conditions associated with pulmonary inflammation, including COPD, asthma, CF and ARDS (e.g., by reducing or alleviating symptoms or severity of ARDS, in particular, by reducing the acute pulmonary inflammatory response, reversing edema, and limiting damage to the lung).

### Cannabidiol (CBD)

“Cannabidiol” or “CBD” is a cannabinoid produced by plants of the genus Cannabis. CBD has antagonist activity on agonists of the CBT and CB2 receptors and acts as an inverse agonist of the CBT and CB2 receptors.

CBD is synthesised in cannabis plants as cannabidiolic acid (CBDA), which decarboxylates to CBD (Table 2). While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying and/or heating the plant material. Persons skilled in the art would be familiar with methods by which decarboxylation of CBDA can be promoted, illustrative examples of which include air-drying, combustion, vaporisation, curing, heating and baking. The decarboxylated CBD will typically bind to and/or stimulate, directly or indirectly, cannabinoid receptors including CB1 and/or CB2.

TABLE 2
Cannabidiol and related cannabinoids

| | | Chemical |
|---|---|---|
| | | properties/ |
| | | [M + H]⁺ ESI |
| Name | Structure | MS |
| cannabidiol (CBD) | 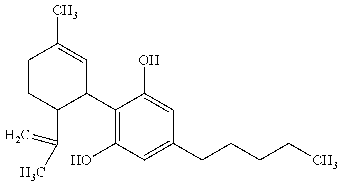 [SMILES: C=C(C)C1CCC(C)=CC1c1c(O)cc(CCCCC)cc1O] | decarboxylation product of CBDA m/z 315.2319 |
| cannabidiolic acid (CBDA) | 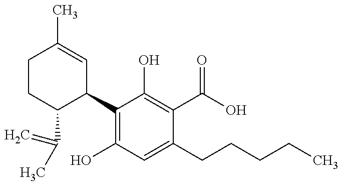 [SMILES: C=C(C)[C@@H]1CCC(C)=C[C@H]1c1c(O)cc(CCCCC)c(C(=O)O)c1O] | m/z 359.2217 |
| cannabigerolic acid (CBGA) | 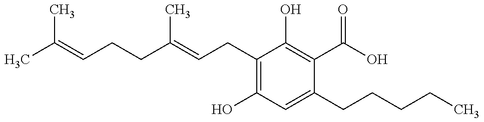 [SMILES: CCCCCc1cc(O)c(C/C=C(\C)CCC=C(C)C)c(O)c1C(=O)O] | m/z 361.2373 |

CBD may be extracted from any suitable plant parts including leaves, flowers or stems and may be produced by any suitable means known to those skilled in the art. For example, CBD extracts may be produced by extraction with supercritical or subcritical C02, or by volatilisation of plant material with a heated gas. Illustrative examples of methods used the extract CBD and other cannabinoids from plant material include the methods described in U.S. patent Ser. No. 10/189,762 and WO 2004/016277.

In an embodiment, the CBD described herein is a synthetic compound.

Synthesised CBD is particularly useful for pharmaceutical development it is largely free from contaminants. A number of methods for the synthesis of CBD are known in the art, illustrative examples of which include methods for the synthesis of CBD as described in U.S. Pat. No. 10,059,682.

The present disclosure further contemplates the use of pharmaceutically acceptable salts of CBD. Suitable pharmaceutically acceptable salts of CBD would be known to persons skilled in the art, illustrative examples of which include salts or esters prepared from pharmaceutically acceptable non-toxic bases or acids, including inorganic bases or acids and organic bases or acids, which would be known to persons skilled in the art.

### Hydroxychloroquine

“Hydroxychloroquine” is a chemical derivative of chloroquine, which features a hydroxyethyl group instead of an ethyl group. Hydroxychloroquine, commonly referred to by the trade name “Plaquenil®” is known to be effective for the treatment of malaria, and has shown efficacy for the treatment of systemic lupus erythematosus, rheumatoid arthritis and Sjögren's Syndrome. Functionally, hydroxychloroquine increases lysosomal pH in antigen presenting cells, and has been demonstrated to inhibit or block the activation of toll-like receptors on plasmacytoid dendritic cells.

As used herein, the term “hydroxychloroquine” includes the racemic hydroxychloroquine, which is 2-[[4-[(7-chloro-4-quinolinyl)amino]pentyl]ethyl]amino]ethanol as disclosed in U.S. Pat. No. 2,546,658, or any of the single enantiomers “(S)-(+) hydroxychloroquine” or “(R)-(−) hydroxychloroquine” as disclosed in U.S. Pat. No. 5,314,894. This term may relate either to the free form of hydroxychloroquine or to any pharmaceutically acceptable salt thereof, such as hydroxychloroquine sulfate.

The present disclosure further contemplates the use of pharmaceutically acceptable salts of hydroxychloroquine. Suitable pharmaceutically acceptable salts would be known to person skilled in the art, illustrative examples of which include salts or esters prepared from pharmaceutically acceptable non-toxic bases or acids, including inorganic bases or acids and organic bases or acids, which would be well known to person skilled in the art.

In an embodiment, the pharmaceutically acceptable salt is hydroxychloroquine sulfate.

### Methods for the Treatment or Prevention of an Inflammatory Condition

The terms “treat”, “treating”, “treatment” and the like are used interchangeably herein to mean relieving, reducing, alleviating, ameliorating or otherwise inhibiting the severity of one or more symptoms of an inflammatory condition in a subject. It is to be understood that the terms “treat”, “treating”, “treatment” and the like, as used herein, do not imply that a subject is treated until the inflammatory condition has been eliminated or are no longer evident. Said treatment may also reduce the severity of the one or more symptoms of an inflammatory condition.

The terms “prevent”, “preventing”, “prevention” and the like are used interchangeably herein to mean preventing the establishment of a condition of a disease, or to otherwise prevent, hinder, retard, abrogate or reverse the onset or progression of a condition or disease or other undesirable symptoms in any way whatsoever.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human.

It is to be understood that the CBD or a pharmaceutically acceptable salt thereof, and the hydroxychloroquine or a pharmaceutically acceptable salt thereof, will be administered to the subject in need thereof in a therapeutically effective amount. As used herein, the terms “therapeutically effective amount” or “effective amount” typically refer to an amount of CBD and an amount of hydroxychloroquine that is sufficient to affect one or more beneficial or desired therapeutic outcomes (e.g., reduction in inflammatory mediators, reduction in white blood cell count and/or neutrophil infiltration, reduction in acute pulmonary inflammatory response, reversal of edema, and limiting damage to the lung). Said beneficial or desired therapeutic outcomes may be quantified by measuring clinical parameters, illustrative examples of which include the measurement of oxygenation index (OI[F_{IO2}×mean airway pressure×100)/Pao₂]) or oxygenation saturation index (OSI[F_{IO2}×mean airway pressure×100)/oxygen saturation by pulse oximetry (Spo₂] as described by Des Prez et al. (2017, Chest, 152(6): 1151-1158), determination of the ratio of arterial oxygen tension and fraction of inspired oxygen (PaO₂:FiO₂) as described by The ARDS Definition Task Force (2012, supra), detection of bilateral pulmonary opacities using chest radiography, detection of altered levels of immunological biomarkers as described by Blondonnet et al. (2016, supra), endoscopic and histological evaluation of colonic mucosa and stool analysis (including fecal occult blood score and stool consistency). Subjective measures of said beneficial or desired therapeutic outcomes can also be made using clinical instruments known in the art, illustrative examples of this include the Lung Injury Score (LIS) (Murray et al., 1988, American Review of Respiratory Disease, 138(3): 720) the American-European Consensus Conference (AECC) Definition (Bernard et al., 1994, American Journal of Respiratory and Critical Care Medicine, 149: 818-824), illness severity scores (e.g., Acute Physiology and Chronic Health Evaluation II (APACHE II) and Simplified Index Score II), the Berlin Criteria (The ARDS Definition Task Force, 2012, supra), Crohn's Disease Activity Index (CDAI; Best et al., 1976, Gastroenterology, 70: 439-444) and Mayo Score for Ulcerative Colitis (UC; Lewis et al., 2008, Inflammatory Bowel Disease, 14: 1660-1666)

Changes in the symptoms or severity of an inflammatory condition as measured by any of the quantitative methods or clinical instruments described elsewhere herein may be expressed using any appropriate statistical measure to demonstrate the magnitude of the reduction in the symptoms or severity of an inflammatory condition. In an embodiment, the methods disclosed herein reduce in the symptoms or severity of an inflammatory condition by at least 10%, preferably at least 20%, preferably at least 30%, preferably at least 40%, preferably at least 50%, preferably at least 60%, preferably at least 70%, preferably at least 80%, preferably at least 90%, or more preferably at least 100% as compared to a subject with the same inflammatory condition who has not been administered CBD and hydroxychloroquine.

An effective amount can be provided in one or more administrations. The exact amount required may vary depending on factors such as the nature and severity of the inflammatory condition to be treated, the age and general health of the subject, and the form in which the active agents are to be administered.

In an embodiment, the hydroxychloroquine or a pharmaceutically acceptable salt thereof is administered at a dose of from about 1 mg to about 1000 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 7 mg, 71 mg, 712 mg, 713 mg, 714 mg, 715 mg, 716 mg, 77 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, or 1000 mg).

Thus, in an embodiment the hydroxychloroquine or a pharmaceutically acceptable salt thereof is administered at a dose of from about 1 mg to about 1000 mg, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, preferably about 100 mg, preferably about 101 mg, preferably about 102 mg, preferably about 103 mg, preferably about 104 mg, preferably about 105 mg, preferably about 106 mg, preferably about 107 mg, preferably about 108 mg, preferably about 109 mg, preferably about 110 mg, preferably about 111 mg, preferably about 112 mg, preferably about 113 mg, preferably about 114 mg, preferably about 115 mg, preferably about 116 mg, preferably about 117 mg, preferably about 118 mg, preferably about 119 mg, preferably about 120 mg, preferably about 121 mg, preferably about 122 mg, preferably about 123 mg, preferably about 124 mg, preferably about 125 mg, preferably about 126 mg, preferably about 127 mg, preferably about 128 mg, preferably about 129 mg, preferably about 130 mg, preferably about 131 mg, preferably about 132 mg, preferably about 131 mg, preferably about 134 mg, preferably about 135 mg, preferably about 136 mg, preferably about 137 mg, preferably about 138 mg, preferably about 139 mg, preferably about 140 mg, preferably about 141 mg, preferably about 142 mg, preferably about 143 mg, preferably about 144 mg, preferably about 145 mg, preferably about 146 mg, preferably about 147 mg, preferably about 148 mg, preferably about 149 mg, preferably about 150 mg, preferably about 151 mg, preferably about 152 mg, preferably about 153 mg, preferably about 154 mg, preferably about 155 mg, preferably about 156 mg, preferably about 157 mg, preferably about 158 mg, preferably about 159 mg, preferably about 160 mg, preferably about 161 mg, preferably about 162 mg, preferably about 163 mg, preferably about 164 mg, preferably about 165 mg, preferably about 166 mg, preferably about 167 mg, preferably about 168 mg, preferably about 169 mg, preferably about 170 mg, preferably about 171 mg, preferably about 172 mg, preferably about 173 mg, preferably about 174 mg, preferably about 175 mg, preferably about 176 mg, preferably about 177 mg, preferably about 178 mg, preferably about 179 mg, preferably about 180 mg, preferably about 181 mg, preferably about 182 mg, preferably about 183 mg, preferably about 184 mg, preferably about 185 mg, preferably about 186 mg, preferably about 187 mg, preferably about 188 mg, preferably about 189 mg, preferably about 190 mg, preferably about 191 mg, preferably about 192 mg, preferably about 193 mg, preferably about 194 mg, preferably about 195 mg, preferably about 196 mg, preferably about 197 mg, preferably about 198 mg, preferably about 199 mg, preferably about 200 mg, preferably about 201 mg, preferably about 202 mg, preferably about 203 mg, preferably about 204 mg, preferably about 205 mg, preferably about 206 mg, preferably about 207 mg, preferably about 208 mg, preferably about 209 mg, preferably about 210 mg, preferably about 211 mg, preferably about 212 mg, preferably about 213 mg, preferably about 214 mg, preferably about 215 mg, preferably about 216 mg, preferably about 217 mg, preferably about 218 mg, preferably about 219 mg, preferably about 220 mg, preferably about 221 mg, preferably about 222 mg, preferably about 223 mg, preferably about 224 mg, preferably about 225 mg, preferably about 226 mg, preferably about 227 mg, preferably about 228 mg, preferably about 229 mg, preferably about 230 mg, preferably about 231 mg, preferably about 232 mg, preferably about 233 mg, preferably about 234 mg, preferably about 235 mg, preferably about 236 mg, preferably about 237 mg, preferably about 238 mg, preferably about 239 mg, preferably about 240 mg, preferably about 241 mg, preferably about 242 mg, preferably about 243 mg, preferably about 244 mg, preferably about 245 mg, preferably about 246 mg, preferably about 247 mg, preferably about 248 mg, preferably about 249 mg, preferably about 250 mg, preferably about 251 mg, preferably about 252 mg, preferably about 253 mg, preferably about 254 mg, preferably about 255 mg, preferably about 256 mg, preferably about 257 mg, preferably about 258 mg, preferably about 259 mg, preferably about 260 mg, preferably about 261 mg, preferably about 262 mg, preferably about 263 mg, preferably about 264 mg, preferably about 265 mg, preferably about 266 mg, preferably about 267 mg, preferably about 268 mg, preferably about 269 mg, preferably about 270 mg, preferably about 271 mg, preferably about 272 mg, preferably about 273 mg, preferably about 274 mg, preferably about 275 mg, preferably about 276 mg, preferably about 277 mg, preferably about 278 mg, preferably about 279 mg, preferably about 280 mg, preferably about 281 mg, preferably about 282 mg, preferably about 283 mg, preferably about 284 mg, preferably about 285 mg, preferably about 286 mg, preferably about 287 mg, preferably about 288 mg, preferably about 289 mg, preferably about 290 mg, preferably about 291 mg, preferably about 292 mg, preferably about 293 mg, preferably about 294 mg, preferably about 295 mg, preferably about 296 mg, preferably about 297 mg, preferably about 298 mg, preferably about 299 mg, preferably about 300 mg, preferably about 301 mg, preferably about 302 mg, preferably about 303 mg, preferably about 304 mg, preferably about 305 mg, preferably about 306 mg, preferably about 307 mg, preferably about 308 mg, preferably about 309 mg, preferably about 310 mg, preferably about 311 mg, preferably about 312 mg, preferably about 313 mg, preferably about 314 mg, preferably about 315 mg, preferably about 316 mg, preferably about 317 mg, preferably about 318 mg, preferably about 319 mg, preferably about 320 mg, preferably about 321 mg, preferably about 322 mg, preferably about 323 mg, preferably about 324 mg, preferably about 325 mg, preferably about 326 mg, preferably about 327 mg, preferably about 328 mg, preferably about 329 mg, preferably about 330 mg, preferably about 331 mg, preferably about 332 mg, preferably about 333 mg, preferably about 334 mg, preferably about 335 mg, preferably about 336 mg, preferably about 337 mg, preferably about 338 mg, preferably about 339 mg, preferably about 340 mg, preferably about 341 mg, preferably about 342 mg, preferably about 343 mg, preferably about 344 mg, preferably about 345 mg, preferably about 346 mg, preferably about 347 mg, preferably about 348 mg, preferably about 349 mg, preferably about 350 mg, preferably about 351 mg, preferably about 352 mg, preferably about 353 mg, preferably about 354 mg, preferably about 355 mg, preferably about 356 mg, preferably about 357 mg, preferably about 358 mg, preferably about 359 mg, preferably about 360 mg, preferably about 361 mg, preferably about 362 mg, preferably about 363 mg, preferably about 364 mg, preferably about 365 mg, preferably about 366 mg, preferably about 367 mg, preferably about 368 mg, preferably about 369 mg, preferably about 370 mg, preferably about 371 mg, preferably about 372 mg, preferably about 373 mg, preferably about 374 mg, preferably about 375 mg, preferably about 376 mg, preferably about 377 mg, preferably about 378 mg, preferably about 379 mg, preferably about 380 mg, preferably about 381 mg, preferably about 382 mg, preferably about 383 mg, preferably about 384 mg, preferably about 385 mg, preferably about 386 mg, preferably about 387 mg, preferably about 388 mg, preferably about 389 mg, preferably about 390 mg, preferably about 391 mg, preferably about 392 mg, preferably about 393 mg, preferably about 394 mg, preferably about 395 mg, preferably about 396 mg, preferably about 397 mg, preferably about 398 mg, preferably about 399 mg, preferably about 400 mg, preferably about 401 mg, preferably about 402 mg, preferably about 403 mg, preferably about 404 mg, preferably about 405 mg, preferably about 406 mg, preferably about 407 mg, preferably about 408 mg, preferably about 409 mg, preferably about 410 mg, preferably about 411 mg, preferably about 412 mg, preferably about 413 mg, preferably about 414 mg, preferably about 415 mg, preferably about 416 mg, preferably about 417 mg, preferably about 418 mg, preferably about 419 mg, preferably about 420 mg, preferably about 421 mg, preferably about 422 mg, preferably about 423 mg, preferably about 424 mg, preferably about 425 mg, preferably about 426 mg, preferably about 427 mg, preferably about 428 mg, preferably about 429 mg, preferably about 430 mg, preferably about 431 mg, preferably about 432 mg, preferably about 433 mg, preferably about 434 mg, preferably about 435 mg, preferably about 436 mg, preferably about 437 mg, preferably about 438 mg, preferably about 439 mg, preferably about 440 mg, preferably about 441 mg, preferably about 442 mg, preferably about 443 mg, preferably about 444 mg, preferably about 445 mg, preferably about 446 mg, preferably about 447 mg, preferably about 448 mg, preferably about 449 mg, preferably about 450 mg, preferably about 451 mg, preferably about 452 mg, preferably about 453 mg, preferably about 454 mg, preferably about 455 mg, preferably about 456 mg, preferably about 457 mg, preferably about 458 mg, preferably about 459 mg, preferably about 460 mg, preferably about 461 mg, preferably about 462 mg, preferably about 463 mg, preferably about 464 mg, preferably about 465 mg, preferably about 466 mg, preferably about 467 mg, preferably about 468 mg, preferably about 469 mg, preferably about 470 mg, preferably about 471 mg, preferably about 472 mg, preferably about 473 mg, preferably about 474 mg, preferably about 475 mg, preferably about 476 mg, preferably about 477 mg, preferably about 478 mg, preferably about 479 mg, preferably about 480 mg, preferably about 481 mg, preferably about 482 mg, preferably about 483 mg, preferably about 484 mg, preferably about 485 mg, preferably about 486 mg, preferably about 487 mg, preferably about 488 mg, preferably about 489 mg, preferably about 490 mg, preferably about 491 mg, preferably about 492 mg, preferably about 493 mg, preferably about 494 mg, preferably about 495 mg, preferably about 496 mg, preferably about 497 mg, preferably about 498 mg, preferably about 499 mg, preferably about 500 mg, preferably about 501 mg, preferably about 502 mg, preferably about 503 mg, preferably about 504 mg, preferably about 505 mg, preferably about 506 mg, preferably about 507 mg, preferably about 508 mg, preferably about 509 mg, preferably about 510 mg, preferably about 511 mg, preferably about 512 mg, preferably about 513 mg, preferably about 514 mg, preferably about 515 mg, preferably about 516 mg, preferably about 517 mg, preferably about 518 mg, preferably about 519 mg, preferably about 520 mg, preferably about 521 mg, preferably about 522 mg, preferably about 523 mg, preferably about 524 mg, preferably about 525 mg, preferably about 526 mg, preferably about 527 mg, preferably about 528 mg, preferably about 529 mg, preferably about 530 mg, preferably about 531 mg, preferably about 532 mg, preferably about 533 mg, preferably about 534 mg, preferably about 535 mg, preferably about 536 mg, preferably about 537 mg, preferably about 538 mg, preferably about 539 mg, preferably about 540 mg, preferably about 541 mg, preferably about 542 mg, preferably about 543 mg, preferably about 544 mg, preferably about 545 mg, preferably about 546 mg, preferably about 547 mg, preferably about 548 mg, preferably about 549 mg, preferably about 550 mg, preferably about 551 mg, preferably about 552 mg, preferably about 553 mg, preferably about 554 mg, preferably about 555 mg, preferably about 556 mg, preferably about 557 mg, preferably about 558 mg, preferably about 559 mg, preferably about 560 mg, preferably about 561 mg, preferably about 562 mg, preferably about 563 mg, preferably about 564 mg, preferably about 565 mg, preferably about 566 mg, preferably about 567 mg, preferably about 568 mg, preferably about 569 mg, preferably about 570 mg, preferably about 571 mg, preferably about 572 mg, preferably about 573 mg, preferably about 574 mg, preferably about 575 mg, preferably about 576 mg, preferably about 577 mg, preferably about 578 mg, preferably about 579 mg, preferably about 580 mg, preferably about 581 mg, preferably about 582 mg, preferably about 583 mg, preferably about 584 mg, preferably about 585 mg, preferably about 586 mg, preferably about 587 mg, preferably about 588 mg, preferably about 589 mg, preferably about 590 mg, preferably about 591 mg, preferably about 592 mg, preferably about 593 mg, preferably about 594 mg, preferably about 595 mg, preferably about 596 mg, preferably about 597 mg, preferably about 598 mg, preferably about 599 mg, preferably about 600 mg, preferably about 601 mg, preferably about 602 mg, preferably about 603 mg, preferably about 604 mg, preferably about 605 mg, preferably about 606 mg, preferably about 607 mg, preferably about 608 mg, preferably about 609 mg, preferably about 610 mg, preferably about 611 mg, preferably about 612 mg, preferably about 613 mg, preferably about 614 mg, preferably about 615 mg, preferably about 616 mg, preferably about 617 mg, preferably about 618 mg, preferably about 619 mg, preferably about 620 mg, preferably about 621 mg, preferably about 622 mg, preferably about 623 mg, preferably about 624 mg, preferably about 625 mg, preferably about 626 mg, preferably about 627 mg, preferably about 628 mg, preferably about 629 mg, preferably about 630 mg, preferably about 631 mg, preferably about 632 mg, preferably about 633 mg, preferably about 634 mg, preferably about 635 mg, preferably about 636 mg, preferably about 637 mg, preferably about 638 mg, preferably about 639 mg, preferably about 640 mg, preferably about 641 mg, preferably about 642 mg, preferably about 643 mg, preferably about 644 mg, preferably about 645 mg, preferably about 646 mg, preferably about 647 mg, preferably about 648 mg, preferably about 649 mg, preferably about 650 mg, preferably about 651 mg, preferably about 652 mg, preferably about 653 mg, preferably about 654 mg, preferably about 655 mg, preferably about 656 mg, preferably about 657 mg, preferably about 658 mg, preferably about 659 mg, preferably about 660 mg, preferably about 661 mg, preferably about 662 mg, preferably about 663 mg, preferably about 664 mg, preferably about 665 mg, preferably about 666 mg, preferably about 667 mg, preferably about 668 mg, preferably about 669 mg, preferably about 670 mg, preferably about 671 mg, preferably about 672 mg, preferably about 673 mg, preferably about 674 mg, preferably about 675 mg, preferably about 676 mg, preferably about 677 mg, preferably about 678 mg, preferably about 679 mg, preferably about 680 mg, preferably about 681 mg, preferably about 682 mg, preferably about 683 mg, preferably about 684 mg, preferably about 685 mg, preferably about 686 mg, preferably about 687 mg, preferably about 688 mg, preferably about 689 mg, preferably about 690 mg, preferably about 691 mg, preferably about 692 mg, preferably about 693 mg, preferably about 694 mg, preferably about 695 mg, preferably about 696 mg, preferably about 697 mg, preferably about 698 mg, preferably about 699 mg, preferably about 700 mg, preferably about 701 mg, preferably about 702 mg, preferably about 703 mg, preferably about 704 mg, preferably about 705 mg, preferably about 706 mg, preferably about 707 mg, preferably about 708 mg, preferably about 709 mg, preferably about 710 mg, preferably about 711 mg, preferably about 712 mg, preferably about 713 mg, preferably about 714 mg, preferably about 715 mg, preferably about 716 mg, preferably about 717 mg, preferably about 718 mg, preferably about 719 mg, preferably about 720 mg, preferably about 721 mg, preferably about 722 mg, preferably about 723 mg, preferably about 724 mg, preferably about 725 mg, preferably about 726 mg, preferably about 727 mg, preferably about 728 mg, preferably about 729 mg, preferably about 730 mg, preferably about 731 mg, preferably about 732 mg, preferably about 733 mg, preferably about 734 mg, preferably about 735 mg, preferably about 736 mg, preferably about 737 mg, preferably about 738 mg, preferably about 739 mg, preferably about 740 mg, preferably about 741 mg, preferably about 742 mg, preferably about 743 mg, preferably about 744 mg, preferably about 745 mg, preferably about 746 mg, preferably about 747 mg, preferably about 748 mg, preferably about 749 mg, preferably about 750 mg, preferably about 751 mg, preferably about 752 mg, preferably about 753 mg, preferably about 754 mg, preferably about 755 mg, preferably about 756 mg, preferably about 757 mg, preferably about 758 mg, preferably about 759 mg, preferably about 760 mg, preferably about 761 mg, preferably about 762 mg, preferably about 763 mg, preferably about 764 mg, preferably about 765 mg, preferably about 766 mg, preferably about 767 mg, preferably about 768 mg, preferably about 769 mg, preferably about 770 mg, preferably about 771 mg, preferably about 772 mg, preferably about 773 mg, preferably about 774 mg, preferably about 775 mg, preferably about 776 mg, preferably about 777 mg, preferably about 778 mg, preferably about 779 mg, preferably about 780 mg, preferably about 781 mg, preferably about 782 mg, preferably about 783 mg, preferably about 784 mg, preferably about 785 mg, preferably about 786 mg, preferably about 787 mg, preferably about 788 mg, preferably about 789 mg, preferably about 790 mg, preferably about 791 mg, preferably about 792 mg, preferably about 793 mg, preferably about 794 mg, preferably about 795 mg, preferably about 796 mg, preferably about 797 mg, preferably about 798 mg, preferably about 799 mg, preferably about 800 mg, preferably about 801 mg, preferably about 802 mg, preferably about 803 mg, preferably about 804 mg, preferably about 805 mg, preferably about 806 mg, preferably about 807 mg, preferably about 808 mg, preferably about 809 mg, preferably about 810 mg, preferably about 811 mg, preferably about 812 mg, preferably about 813 mg, preferably about 814 mg, preferably about 815 mg, preferably about 816 mg, preferably about 817 mg, preferably about 818 mg, preferably about 819 mg, preferably about 820 mg, preferably about 821 mg, preferably about 822 mg, preferably about 823 mg, preferably about 824 mg, preferably about 825 mg, preferably about 826 mg, preferably about 827 mg, preferably about 828 mg, preferably about 829 mg, preferably about 830 mg, preferably about 831 mg, preferably about 832 mg, preferably about 833 mg, preferably about 834 mg, preferably about 835 mg, preferably about 836 mg, preferably about 837 mg, preferably about 838 mg, preferably about 839 mg, preferably about 840 mg, preferably about 841 mg, preferably about 842 mg, preferably about 843 mg, preferably about 844 mg, preferably about 845 mg, preferably about 846 mg, preferably about 847 mg, preferably about 848 mg, preferably about 849 mg, preferably about 850 mg, preferably about 851 mg, preferably about 852 mg, preferably about 853 mg, preferably about 854 mg, preferably about 855 mg, preferably about 856 mg, preferably about 857 mg, preferably about 858 mg, preferably about 859 mg, preferably about 860 mg, preferably about 861 mg, preferably about 862 mg, preferably about 863 mg, preferably about 864 mg, preferably about 865 mg, preferably about 866 mg, preferably about 867 mg, preferably about 868 mg, preferably about 869 mg, preferably about 870 mg, preferably about 871 mg, preferably about 872 mg, preferably about 873 mg, preferably about 874 mg, preferably about 875 mg, preferably about 876 mg, preferably about 877 mg, preferably about 878 mg, preferably about 879 mg, preferably about 880 mg, preferably about 881 mg, preferably about 882 mg, preferably about 883 mg, preferably about 884 mg, preferably about 885 mg, preferably about 886 mg, preferably about 887 mg, preferably about 888 mg, preferably about 889 mg, preferably about 890 mg, preferably about 891 mg, preferably about 892 mg, preferably about 893 mg, preferably about 894 mg, preferably about 895 mg, preferably about 896 mg, preferably about 897 mg, preferably about 898 mg, preferably about 899 mg, preferably about 900 mg, preferably about 901 mg, preferably about 902 mg, preferably about 903 mg, preferably about 904 mg, preferably about 905 mg, preferably about 906 mg, preferably about 907 mg, preferably about 908 mg, preferably about 909 mg, preferably about 910 mg, preferably about 911 mg, preferably about 912 mg, preferably about 913 mg, preferably about 914 mg, preferably about 915 mg, preferably about 916 mg, preferably about 917 mg, preferably about 918 mg, preferably about 919 mg, preferably about 920 mg, preferably about 921 mg, preferably about 922 mg, preferably about 923 mg, preferably about 924 mg, preferably about 925 mg, preferably about 926 mg, preferably about 927 mg, preferably about 928 mg, preferably about 929 mg, preferably about 930 mg, preferably about 931 mg, preferably about 932 mg, preferably about 933 mg, preferably about 934 mg, preferably about 935 mg, preferably about 936 mg, preferably about 937 mg, preferably about 938 mg, preferably about 939 mg, preferably about 940 mg, preferably about 941 mg, preferably about 942 mg, preferably about 943 mg, preferably about 944 mg, preferably about 945 mg, preferably about 946 mg, preferably about 947 mg, preferably about 948 mg, preferably about 949 mg, preferably about 950 mg, preferably about 951 mg, preferably about 952 mg, preferably about 953 mg, preferably about 954 mg, preferably about 955 mg, preferably about 956 mg, preferably about 957 mg, preferably about 958 mg, preferably about 959 mg, preferably about 960 mg, preferably about 961 mg, preferably about 962 mg, preferably about 963 mg, preferably about 964 mg, preferably about 965 mg, preferably about 966 mg, preferably about 967 mg, preferably about 968 mg, preferably about 969 mg, preferably about 970 mg, preferably about 971 mg, preferably about 972 mg, preferably about 973 mg, preferably about 974 mg, preferably about 975 mg, preferably about 976 mg, preferably about 977 mg, preferably about 978 mg, preferably about 979 mg, preferably about 980 mg, preferably about 981 mg, preferably about 982 mg, preferably about 983 mg, preferably about 984 mg, preferably about 985 mg, preferably about 986 mg, preferably about 987 mg, preferably about 988 mg, preferably about 989 mg, preferably about 990 mg, preferably about 991 mg, preferably about 992 mg, preferably about 993 mg, preferably about 994 mg, preferably about 995 mg, preferably about 996 mg, preferably about 997 mg, preferably about 998 mg, preferably about 999 mg, or more preferably about 1000 mg.

In an embodiment, the hydroxychloroquine or a pharmaceutically acceptable salt thereof is administered at an aforementioned dose twice per day.

In an embodiment, the hydroxychloroquine or a pharmaceutically acceptable salt thereof is administered at a dose of at least about 1 mg, twice per day.

In an embodiment, the hydroxychloroquine or a pharmaceutically acceptable salt thereof is administered at a dose from about 1 mg to about 500 mg, twice per day.

In an embodiment, the hydroxychloroquine or a pharmaceutically acceptable salt thereof is administered at a dose of from about 200 mg to about 400 mg, twice per day.

In an embodiment, the hydroxychloroquine or a pharmaceutically acceptable salt thereof is administered at a loading dose of at least about 1 mg, twice per day.

In an embodiment, the hydroxychloroquine or a pharmaceutically acceptable salt thereof is administered at a loading dose of from about 1 mg to about 500 mg, twice per day.

In an embodiment, the hydroxychloroquine or a pharmaceutically acceptable salt thereof is administered at a loading dose of about 400 mg, twice per day.

In an embodiment, the hydroxychloroquine or a pharmaceutically acceptable salt thereof is administered at a maintenance dose of at least about 1 mg, twice per day.

In an embodiment, the hydroxychloroquine or a pharmaceutically acceptable salt thereof is administered at a maintenance dose from about 1 mg to about 500 mg, twice per day.

In an embodiment, the hydroxychloroquine or a pharmaceutically acceptable salt thereof is administered at a maintenance dose of about 200 mg, twice per day.

In an embodiment, the CBD or a pharmaceutically acceptable salt thereof is administered at a dose of from about 1 mg to about 1500 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, 1000 mg, 1100 mg, 1101 mg, 1102 mg, 1103 mg, 1104 mg, 1105 mg, 1106 mg, 1107 mg, 1108 mg, 1109 mg, 1110 mg, 1111 mg, 1112 mg, 1113 mg, 1114 mg, 1115 mg, 1116 mg, 1117 mg, 1118 mg, 1119 mg, 1120 mg, 1121 mg, 1122 mg, 1123 mg, 1124 mg, 1125 mg, 1126 mg, 1127 mg, 1128 mg, 1129 mg, 1130 mg, 1131 mg, 1132 mg, 1133 mg, 1134 mg, 1135 mg, 1136 mg, 1137 mg, 1138 mg, 1139 mg, 1140 mg, 1141 mg, 1142 mg, 1143 mg, 1144 mg, 1145 mg, 1146 mg, 1147 mg, 1148 mg, 1149 mg, 1150 mg, 1151 mg, 1152 mg, 1153 mg, 1154 mg, 1155 mg, 1156 mg, 1157 mg, 1158 mg, 1159 mg, 1160 mg, 1161 mg, 1162 mg, 1163 mg, 1164 mg, 1165 mg, 1166 mg, 1167 mg, 1168 mg, 1169 mg, 1170 mg, 1171 mg, 1172 mg, 1173 mg, 1174 mg, 1175 mg, 1176 mg, 1177 mg, 1178 mg, 1179 mg, 1180 mg, 1181 mg, 1182 mg, 1183 mg, 1184 mg, 1185 mg, 1186 mg, 1187 mg, 1188 mg, 1189 mg, 1190 mg, 1191 mg, 1192 mg, 1193 mg, 1194 mg, 1195 mg, 1196 mg, 1197 mg, 1198 mg, 1199 mg, 1200 mg, 1201 mg, 1202 mg, 1203 mg, 1204 mg, 1205 mg, 1206 mg, 1207 mg, 1208 mg, 1209 mg, 1210 mg, 1211 mg, 1212 mg, 1213 mg, 1214 mg, 1215 mg, 1216 mg, 1217 mg, 1218 mg, 1219 mg, 1220 mg, 1221 mg, 1222 mg, 1223 mg, 1224 mg, 1225 mg, 1226 mg, 1227 mg, 1228 mg, 1229 mg, 1230 mg, 1231 mg, 1232 mg, 1233 mg, 1234 mg, 1235 mg, 1236 mg, 1237 mg, 1238 mg, 239 mg, 1240 mg, 1241 mg, 1242 mg, 1243 mg, 1244 mg, 1245 mg, 1246 mg, 1247 mg, 1248 mg, 1249 mg, 1250 mg, 1251 mg, 1252 mg, 1253 mg, 1254 mg, 1255 mg, 1256 mg, 1257 mg, 1258 mg, 1259 mg, 1260 mg, 1261 mg, 1262 mg, 1263 mg, 1264 mg, 1265 mg, 1266 mg, 1267 mg, 1268 mg, 1269 mg, 1270 mg, 1271 mg, 1272 mg, 1273 mg, 1274 mg, 1275 mg, 1276 mg, 1277 mg, 1278 mg, 1279 mg, 1280 mg, 1281 mg, 1282 mg, 1283 mg, 1284 mg, 1285 mg, 1286 mg, 1287 mg, 1288 mg, 1289 mg, 1290 mg, 1291 mg, 1292 mg, 1293 mg, 1294 mg, 1295 mg, 1296 mg, 1297 mg, 1298 mg, 1299 mg, 1300 mg, 1301 mg, 1302 mg, 303 mg, 1304 mg, 1305 mg, 1306 mg, 1307 mg, 1308 mg, 1309 mg, 1310 mg, 1311 mg, 1312 mg, 1313 mg, 1314 mg, 1315 mg, 1316 mg, 1317 mg, 1318 mg, 1319 mg, 1320 mg, 1321 mg, 1322 mg, 1323 mg, 1324 mg, 1325 mg, 1326 mg, 1327 mg, 1328 mg, 1329 mg, 1330 mg, 1331 mg, 1332 mg, 1333 mg, 1334 mg, 1335 mg, 1336 mg, 1337 mg, 1338 mg, 1339 mg, 1340 mg, 1341 mg, 1342 mg, 1343 mg, 1344 mg, 1345 mg, 1346 mg, 1347 mg, 1348 mg, 1349 mg, 1350 mg, 1351 mg, 1352 mg, 1353 mg, 1354 mg, 1355 mg, 1356 mg, 1357 mg, 1358 mg, 1359 mg, 1360 mg, 1361 mg, 1362 mg, 1363 mg, 1364 mg, 1365 mg, 1366 mg, 1367 mg, 1368 mg, 1369 mg, 1370 mg, 1371 mg, 1372 mg, 1373 mg, 1374 mg, 1375 mg, 1376 mg, 1377 mg, 1378 mg, 1379 mg, 1380 mg, 1381 mg, 1382 mg, 1383 mg, 1384 mg, 1385 mg, 1386 mg, 1387 mg, 1388 mg, 1389 mg, 1390 mg, 1391 mg, 1392 mg, 1393 mg, 1394 mg, 1395 mg, 1396 mg, 1397 mg, 1398 mg, 1399 mg, 1400 mg, 1401 mg, 1402 mg, 1403 mg, 1404 mg, 1405 mg, 1406 mg, 1407 mg, 1408 mg, 1409 mg, 1410 mg, 1411 mg, 1412 mg, 1413 mg, 1414 mg, 1415 mg, 1416 mg, 1417 mg, 1418 mg, 1419 mg, 1420 mg, 1421 mg, 1422 mg, 1423 mg, 1424 mg, 1425 mg, 1426 mg, 1427 mg, 1428 mg, 1429 mg, 1430 mg, 1431 mg, 1432 mg, 1433 mg, 1434 mg, 1435 mg, 1436 mg, 1437 mg, 1438 mg, 1439 mg, 1440 mg, 1441 mg, 1442 mg, 1443 mg, 1444 mg, 1445 mg, 1446 mg, 1447 mg, 1448 mg, 1449 mg, 1450 mg, 1451 mg, 1452 mg, 1453 mg, 1454 mg, 1455 mg, 1456 mg, 1457 mg, 1458 mg, 1459 mg, 1460 mg, 1461 mg, 1462 mg, 1463 mg, 1464 mg, 1465 mg, 1466 mg, 1467 mg, 1468 mg, 1469 mg, 1470 mg, 1471 mg, 1472 mg, 1473 mg, 1474 mg, 1475 mg, 1476 mg, 1477 mg, 1478 mg, 1479 mg, 1480 mg, 1481 mg, 1482 mg, 1483 mg, 1484 mg, 1485 mg, 1486 mg, 1487 mg, 1488 mg, 1489 mg, 1490 mg, 1491 mg, 1492 mg, 1493 mg, 1494 mg, 1495 mg, 1496 mg, 1497 mg, 1498 mg, 1499 mg, or 1500 mg).

Thus, in an embodiment the CBD or a pharmaceutically acceptable salt thereof is administered at a dose of from about 1 mg to about 1500 mg, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, preferably about 100 mg, preferably about 101 mg, preferably about 102 mg, preferably about 103 mg, preferably about 104 mg, preferably about 105 mg, preferably about 106 mg, preferably about 107 mg, preferably about 108 mg, preferably about 109 mg, preferably about 110 mg, preferably about 111 mg, preferably about 112 mg, preferably about 113 mg, preferably about 114 mg, preferably about 115 mg, preferably about 116 mg, preferably about 117 mg, preferably about 118 mg, preferably about 119 mg, preferably about 120 mg, preferably about 121 mg, preferably about 122 mg, preferably about 123 mg, preferably about 124 mg, preferably about 125 mg, preferably about 126 mg, preferably about 127 mg, preferably about 128 mg, preferably about 129 mg, preferably about 130 mg, preferably about 131 mg, preferably about 132 mg, preferably about 131 mg, preferably about 134 mg, preferably about 135 mg, preferably about 136 mg, preferably about 137 mg, preferably about 138 mg, preferably about 139 mg, preferably about 140 mg, preferably about 141 mg, preferably about 142 mg, preferably about 143 mg, preferably about 144 mg, preferably about 145 mg, preferably about 146 mg, preferably about 147 mg, preferably about 148 mg, preferably about 149 mg, preferably about 150 mg, preferably about 151 mg, preferably about 152 mg, preferably about 153 mg, preferably about 154 mg, preferably about 155 mg, preferably about 156 mg, preferably about 157 mg, preferably about 158 mg, preferably about 159 mg, preferably about 160 mg, preferably about 161 mg, preferably about 162 mg, preferably about 163 mg, preferably about 164 mg, preferably about 165 mg, preferably about 166 mg, preferably about 167 mg, preferably about 168 mg, preferably about 169 mg, preferably about 170 mg, preferably about 171 mg, preferably about 172 mg, preferably about 173 mg, preferably about 174 mg, preferably about 175 mg, preferably about 176 mg, preferably about 177 mg, preferably about 178 mg, preferably about 179 mg, preferably about 180 mg, preferably about 181 mg, preferably about 182 mg, preferably about 183 mg, preferably about 184 mg, preferably about 185 mg, preferably about 186 mg, preferably about 187 mg, preferably about 188 mg, preferably about 189 mg, preferably about 190 mg, preferably about 191 mg, preferably about 192 mg, preferably about 193 mg, preferably about 194 mg, preferably about 195 mg, preferably about 196 mg, preferably about 197 mg, preferably about 198 mg, preferably about 199 mg, preferably about 200 mg, preferably about 201 mg, preferably about 202 mg, preferably about 203 mg, preferably about 204 mg, preferably about 205 mg, preferably about 206 mg, preferably about 207 mg, preferably about 208 mg, preferably about 209 mg, preferably about 210 mg, preferably about 211 mg, preferably about 212 mg, preferably about 213 mg, preferably about 214 mg, preferably about 215 mg, preferably about 216 mg, preferably about 217 mg, preferably about 218 mg, preferably about 219 mg, preferably about 220 mg, preferably about 221 mg, preferably about 222 mg, preferably about 223 mg, preferably about 224 mg, preferably about 225 mg, preferably about 226 mg, preferably about 227 mg, preferably about 228 mg, preferably about 229 mg, preferably about 230 mg, preferably about 231 mg, preferably about 232 mg, preferably about 233 mg, preferably about 234 mg, preferably about 235 mg, preferably about 236 mg, preferably about 237 mg, preferably about 238 mg, preferably about 239 mg, preferably about 240 mg, preferably about 241 mg, preferably about 242 mg, preferably about 243 mg, preferably about 244 mg, preferably about 245 mg, preferably about 246 mg, preferably about 247 mg, preferably about 248 mg, preferably about 249 mg, preferably about 250 mg, preferably about 251 mg, preferably about 252 mg, preferably about 253 mg, preferably about 254 mg, preferably about 255 mg, preferably about 256 mg, preferably about 257 mg, preferably about 258 mg, preferably about 259 mg, preferably about 260 mg, preferably about 261 mg, preferably about 262 mg, preferably about 263 mg, preferably about 264 mg, preferably about 265 mg, preferably about 266 mg, preferably about 267 mg, preferably about 268 mg, preferably about 269 mg, preferably about 270 mg, preferably about 271 mg, preferably about 272 mg, preferably about 273 mg, preferably about 274 mg, preferably about 275 mg, preferably about 276 mg, preferably about 277 mg, preferably about 278 mg, preferably about 279 mg, preferably about 280 mg, preferably about 281 mg, preferably about 282 mg, preferably about 283 mg, preferably about 284 mg, preferably about 285 mg, preferably about 286 mg, preferably about 287 mg, preferably about 288 mg, preferably about 289 mg, preferably about 290 mg, preferably about 291 mg, preferably about 292 mg, preferably about 293 mg, preferably about 294 mg, preferably about 295 mg, preferably about 296 mg, preferably about 297 mg, preferably about 298 mg, preferably about 299 mg, preferably about 300 mg, preferably about 301 mg, preferably about 302 mg, preferably about 303 mg, preferably about 304 mg, preferably about 305 mg, preferably about 306 mg, preferably about 307 mg, preferably about 308 mg, preferably about 309 mg, preferably about 310 mg, preferably about 311 mg, preferably about 312 mg, preferably about 313 mg, preferably about 314 mg, preferably about 315 mg, preferably about 316 mg, preferably about 317 mg, preferably about 318 mg, preferably about 319 mg, preferably about 320 mg, preferably about 321 mg, preferably about 322 mg, preferably about 323 mg, preferably about 324 mg, preferably about 325 mg, preferably about 326 mg, preferably about 327 mg, preferably about 328 mg, preferably about 329 mg, preferably about 330 mg, preferably about 331 mg, preferably about 332 mg, preferably about 333 mg, preferably about 334 mg, preferably about 335 mg, preferably about 336 mg, preferably about 337 mg, preferably about 338 mg, preferably about 339 mg, preferably about 340 mg, preferably about 341 mg, preferably about 342 mg, preferably about 343 mg, preferably about 344 mg, preferably about 345 mg, preferably about 346 mg, preferably about 347 mg, preferably about 348 mg, preferably about 349 mg, preferably about 350 mg, preferably about 351 mg, preferably about 352 mg, preferably about 353 mg, preferably about 354 mg, preferably about 355 mg, preferably about 356 mg, preferably about 357 mg, preferably about 358 mg, preferably about 359 mg, preferably about 360 mg, preferably about 361 mg, preferably about 362 mg, preferably about 363 mg, preferably about 364 mg, preferably about 365 mg, preferably about 366 mg, preferably about 367 mg, preferably about 368 mg, preferably about 369 mg, preferably about 370 mg, preferably about 371 mg, preferably about 372 mg, preferably about 373 mg, preferably about 374 mg, preferably about 375 mg, preferably about 376 mg, preferably about 377 mg, preferably about 378 mg, preferably about 379 mg, preferably about 380 mg, preferably about 381 mg, preferably about 382 mg, preferably about 383 mg, preferably about 384 mg, preferably about 385 mg, preferably about 386 mg, preferably about 387 mg, preferably about 388 mg, preferably about 389 mg, preferably about 390 mg, preferably about 391 mg, preferably about 392 mg, preferably about 393 mg, preferably about 394 mg, preferably about 395 mg, preferably about 396 mg, preferably about 397 mg, preferably about 398 mg, preferably about 399 mg, preferably about 400 mg, preferably about 401 mg, preferably about 402 mg, preferably about 403 mg, preferably about 404 mg, preferably about 405 mg, preferably about 406 mg, preferably about 407 mg, preferably about 408 mg, preferably about 409 mg, preferably about 410 mg, preferably about 411 mg, preferably about 412 mg, preferably about 413 mg, preferably about 414 mg, preferably about 415 mg, preferably about 416 mg, preferably about 417 mg, preferably about 418 mg, preferably about 419 mg, preferably about 420 mg, preferably about 421 mg, preferably about 422 mg, preferably about 423 mg, preferably about 424 mg, preferably about 425 mg, preferably about 426 mg, preferably about 427 mg, preferably about 428 mg, preferably about 429 mg, preferably about 430 mg, preferably about 431 mg, preferably about 432 mg, preferably about 433 mg, preferably about 434 mg, preferably about 435 mg, preferably about 436 mg, preferably about 437 mg, preferably about 438 mg, preferably about 439 mg, preferably about 440 mg, preferably about 441 mg, preferably about 442 mg, preferably about 443 mg, preferably about 444 mg, preferably about 445 mg, preferably about 446 mg, preferably about 447 mg, preferably about 448 mg, preferably about 449 mg, preferably about 450 mg, preferably about 451 mg, preferably about 452 mg, preferably about 453 mg, preferably about 454 mg, preferably about 455 mg, preferably about 456 mg, preferably about 457 mg, preferably about 458 mg, preferably about 459 mg, preferably about 460 mg, preferably about 461 mg, preferably about 462 mg, preferably about 463 mg, preferably about 464 mg, preferably about 465 mg, preferably about 466 mg, preferably about 467 mg, preferably about 468 mg, preferably about 469 mg, preferably about 470 mg, preferably about 471 mg, preferably about 472 mg, preferably about 473 mg, preferably about 474 mg, preferably about 475 mg, preferably about 476 mg, preferably about 477 mg, preferably about 478 mg, preferably about 479 mg, preferably about 480 mg, preferably about 481 mg, preferably about 482 mg, preferably about 483 mg, preferably about 484 mg, preferably about 485 mg, preferably about 486 mg, preferably about 487 mg, preferably about 488 mg, preferably about 489 mg, preferably about 490 mg, preferably about 491 mg, preferably about 492 mg, preferably about 493 mg, preferably about 494 mg, preferably about 495 mg, preferably about 496 mg, preferably about 497 mg, preferably about 498 mg, preferably about 499 mg, preferably about 500 mg, preferably about 501 mg, preferably about 502 mg, preferably about 503 mg, preferably about 504 mg, preferably about 505 mg, preferably about 506 mg, preferably about 507 mg, preferably about 508 mg, preferably about 509 mg, preferably about 510 mg, preferably about 511 mg, preferably about 512 mg, preferably about 513 mg, preferably about 514 mg, preferably about 515 mg, preferably about 516 mg, preferably about 517 mg, preferably about 518 mg, preferably about 519 mg, preferably about 520 mg, preferably about 521 mg, preferably about 522 mg, preferably about 523 mg, preferably about 524 mg, preferably about 525 mg, preferably about 526 mg, preferably about 527 mg, preferably about 528 mg, preferably about 529 mg, preferably about 530 mg, preferably about 531 mg, preferably about 532 mg, preferably about 533 mg, preferably about 534 mg, preferably about 535 mg, preferably about 536 mg, preferably about 537 mg, preferably about 538 mg, preferably about 539 mg, preferably about 540 mg, preferably about 541 mg, preferably about 542 mg, preferably about 543 mg, preferably about 544 mg, preferably about 545 mg, preferably about 546 mg, preferably about 547 mg, preferably about 548 mg, preferably about 549 mg, preferably about 550 mg, preferably about 551 mg, preferably about 552 mg, preferably about 553 mg, preferably about 554 mg, preferably about 555 mg, preferably about 556 mg, preferably about 557 mg, preferably about 558 mg, preferably about 559 mg, preferably about 560 mg, preferably about 561 mg, preferably about 562 mg, preferably about 563 mg, preferably about 564 mg, preferably about 565 mg, preferably about 566 mg, preferably about 567 mg, preferably about 568 mg, preferably about 569 mg, preferably about 570 mg, preferably about 571 mg, preferably about 572 mg, preferably about 573 mg, preferably about 574 mg, preferably about 575 mg, preferably about 576 mg, preferably about 577 mg, preferably about 578 mg, preferably about 579 mg, preferably about 580 mg, preferably about 581 mg, preferably about 582 mg, preferably about 583 mg, preferably about 584 mg, preferably about 585 mg, preferably about 586 mg, preferably about 587 mg, preferably about 588 mg, preferably about 589 mg, preferably about 590 mg, preferably about 591 mg, preferably about 592 mg, preferably about 593 mg, preferably about 594 mg, preferably about 595 mg, preferably about 596 mg, preferably about 597 mg, preferably about 598 mg, preferably about 599 mg, preferably about 600 mg, preferably about 601 mg, preferably about 602 mg, preferably about 603 mg, preferably about 604 mg, preferably about 605 mg, preferably about 606 mg, preferably about 607 mg, preferably about 608 mg, preferably about 609 mg, preferably about 610 mg, preferably about 611 mg, preferably about 612 mg, preferably about 613 mg, preferably about 614 mg, preferably about 615 mg, preferably about 616 mg, preferably about 617 mg, preferably about 618 mg, preferably about 619 mg, preferably about 620 mg, preferably about 621 mg, preferably about 622 mg, preferably about 623 mg, preferably about 624 mg, preferably about 625 mg, preferably about 626 mg, preferably about 627 mg, preferably about 628 mg, preferably about 629 mg, preferably about 630 mg, preferably about 631 mg, preferably about 632 mg, preferably about 633 mg, preferably about 634 mg, preferably about 635 mg, preferably about 636 mg, preferably about 637 mg, preferably about 638 mg, preferably about 639 mg, preferably about 640 mg, preferably about 641 mg, preferably about 642 mg, preferably about 643 mg, preferably about 644 mg, preferably about 645 mg, preferably about 646 mg, preferably about 647 mg, preferably about 648 mg, preferably about 649 mg, preferably about 650 mg, preferably about 651 mg, preferably about 652 mg, preferably about 653 mg, preferably about 654 mg, preferably about 655 mg, preferably about 656 mg, preferably about 657 mg, preferably about 658 mg, preferably about 659 mg, preferably about 660 mg, preferably about 661 mg, preferably about 662 mg, preferably about 663 mg, preferably about 664 mg, preferably about 665 mg, preferably about 666 mg, preferably about 667 mg, preferably about 668 mg, preferably about 669 mg, preferably about 670 mg, preferably about 671 mg, preferably about 672 mg, preferably about 673 mg, preferably about 674 mg, preferably about 675 mg, preferably about 676 mg, preferably about 677 mg, preferably about 678 mg, preferably about 679 mg, preferably about 680 mg, preferably about 681 mg, preferably about 682 mg, preferably about 683 mg, preferably about 684 mg, preferably about 685 mg, preferably about 686 mg, preferably about 687 mg, preferably about 688 mg, preferably about 689 mg, preferably about 690 mg, preferably about 691 mg, preferably about 692 mg, preferably about 693 mg, preferably about 694 mg, preferably about 695 mg, preferably about 696 mg, preferably about 697 mg, preferably about 698 mg, preferably about 699 mg, preferably about 700 mg, preferably about 701 mg, preferably about 702 mg, preferably about 703 mg, preferably about 704 mg, preferably about 705 mg, preferably about 706 mg, preferably about 707 mg, preferably about 708 mg, preferably about 709 mg, preferably about 710 mg, preferably about 711 mg, preferably about 712 mg, preferably about 713 mg, preferably about 714 mg, preferably about 715 mg, preferably about 716 mg, preferably about 717 mg, preferably about 718 mg, preferably about 719 mg, preferably about 720 mg, preferably about 721 mg, preferably about 722 mg, preferably about 723 mg, preferably about 724 mg, preferably about 725 mg, preferably about 726 mg, preferably about 727 mg, preferably about 728 mg, preferably about 729 mg, preferably about 730 mg, preferably about 731 mg, preferably about 732 mg, preferably about 733 mg, preferably about 734 mg, preferably about 735 mg, preferably about 736 mg, preferably about 737 mg, preferably about 738 mg, preferably about 739 mg, preferably about 740 mg, preferably about 741 mg, preferably about 742 mg, preferably about 743 mg, preferably about 744 mg, preferably about 745 mg, preferably about 746 mg, preferably about 747 mg, preferably about 748 mg, preferably about 749 mg, preferably about 750 mg, preferably about 751 mg, preferably about 752 mg, preferably about 753 mg, preferably about 754 mg, preferably about 755 mg, preferably about 756 mg, preferably about 757 mg, preferably about 758 mg, preferably about 759 mg, preferably about 760 mg, preferably about 761 mg, preferably about 762 mg, preferably about 763 mg, preferably about 764 mg, preferably about 765 mg, preferably about 766 mg, preferably about 767 mg, preferably about 768 mg, preferably about 769 mg, preferably about 770 mg, preferably about 771 mg, preferably about 772 mg, preferably about 773 mg, preferably about 774 mg, preferably about 775 mg, preferably about 776 mg, preferably about 777 mg, preferably about 778 mg, preferably about 779 mg, preferably about 780 mg, preferably about 781 mg, preferably about 782 mg, preferably about 783 mg, preferably about 784 mg, preferably about 785 mg, preferably about 786 mg, preferably about 787 mg, preferably about 788 mg, preferably about 789 mg, preferably about 790 mg, preferably about 791 mg, preferably about 792 mg, preferably about 793 mg, preferably about 794 mg, preferably about 795 mg, preferably about 796 mg, preferably about 797 mg, preferably about 798 mg, preferably about 799 mg, preferably about 800 mg, preferably about 801 mg, preferably about 802 mg, preferably about 803 mg, preferably about 804 mg, preferably about 805 mg, preferably about 806 mg, preferably about 807 mg, preferably about 808 mg, preferably about 809 mg, preferably about 810 mg, preferably about 811 mg, preferably about 812 mg, preferably about 813 mg, preferably about 814 mg, preferably about 815 mg, preferably about 816 mg, preferably about 817 mg, preferably about 818 mg, preferably about 819 mg, preferably about 820 mg, preferably about 821 mg, preferably about 822 mg, preferably about 823 mg, preferably about 824 mg, preferably about 825 mg, preferably about 826 mg, preferably about 827 mg, preferably about 828 mg, preferably about 829 mg, preferably about 830 mg, preferably about 831 mg, preferably about 832 mg, preferably about 833 mg, preferably about 834 mg, preferably about 835 mg, preferably about 836 mg, preferably about 837 mg, preferably about 838 mg, preferably about 839 mg, preferably about 840 mg, preferably about 841 mg, preferably about 842 mg, preferably about 843 mg, preferably about 844 mg, preferably about 845 mg, preferably about 846 mg, preferably about 847 mg, preferably about 848 mg, preferably about 849 mg, preferably about 850 mg, preferably about 851 mg, preferably about 852 mg, preferably about 853 mg, preferably about 854 mg, preferably about 855 mg, preferably about 856 mg, preferably about 857 mg, preferably about 858 mg, preferably about 859 mg, preferably about 860 mg, preferably about 861 mg, preferably about 862 mg, preferably about 863 mg, preferably about 864 mg, preferably about 865 mg, preferably about 866 mg, preferably about 867 mg, preferably about 868 mg, preferably about 869 mg, preferably about 870 mg, preferably about 871 mg, preferably about 872 mg, preferably about 873 mg, preferably about 874 mg, preferably about 875 mg, preferably about 876 mg, preferably about 877 mg, preferably about 878 mg, preferably about 879 mg, preferably about 880 mg, preferably about 881 mg, preferably about 882 mg, preferably about 883 mg, preferably about 884 mg, preferably about 885 mg, preferably about 886 mg, preferably about 887 mg, preferably about 888 mg, preferably about 889 mg, preferably about 890 mg, preferably about 891 mg, preferably about 892 mg, preferably about 893 mg, preferably about 894 mg, preferably about 895 mg, preferably about 896 mg, preferably about 897 mg, preferably about 898 mg, preferably about 899 mg, preferably about 900 mg, preferably about 901 mg, preferably about 902 mg, preferably about 903 mg, preferably about 904 mg, preferably about 905 mg, preferably about 906 mg, preferably about 907 mg, preferably about 908 mg, preferably about 909 mg, preferably about 910 mg, preferably about 911 mg, preferably about 912 mg, preferably about 913 mg, preferably about 914 mg, preferably about 915 mg, preferably about 916 mg, preferably about 917 mg, preferably about 918 mg, preferably about 919 mg, preferably about 920 mg, preferably about 921 mg, preferably about 922 mg, preferably about 923 mg, preferably about 924 mg, preferably about 925 mg, preferably about 926 mg, preferably about 927 mg, preferably about 928 mg, preferably about 929 mg, preferably about 930 mg, preferably about 931 mg, preferably about 932 mg, preferably about 933 mg, preferably about 934 mg, preferably about 935 mg, preferably about 936 mg, preferably about 937 mg, preferably about 938 mg, preferably about 939 mg, preferably about 940 mg, preferably about 941 mg, preferably about 942 mg, preferably about 943 mg, preferably about 944 mg, preferably about 945 mg, preferably about 946 mg, preferably about 947 mg, preferably about 948 mg, preferably about 949 mg, preferably about 950 mg, preferably about 951 mg, preferably about 952 mg, preferably about 953 mg, preferably about 954 mg, preferably about 955 mg, preferably about 956 mg, preferably about 957 mg, preferably about 958 mg, preferably about 959 mg, preferably about 960 mg, preferably about 961 mg, preferably about 962 mg, preferably about 963 mg, preferably about 964 mg, preferably about 965 mg, preferably about 966 mg, preferably about 967 mg, preferably about 968 mg, preferably about 969 mg, preferably about 970 mg, preferably about 971 mg, preferably about 972 mg, preferably about 973 mg, preferably about 974 mg, preferably about 975 mg, preferably about 976 mg, preferably about 977 mg, preferably about 978 mg, preferably about 979 mg, preferably about 980 mg, preferably about 981 mg, preferably about 982 mg, preferably about 983 mg, preferably about 984 mg, preferably about 985 mg, preferably about 986 mg, preferably about 987 mg, preferably about 988 mg, preferably about 989 mg, preferably about 990 mg, preferably about 991 mg, preferably about 992 mg, preferably about 993 mg, preferably about 994 mg, preferably about 995 mg, preferably about 996 mg, preferably about 997 mg, preferably about 998 mg, preferably about 999 mg, preferably about 1000 mg, preferably about 1100 mg, preferably about 1101 mg, preferably about 1102 mg, preferably about 1103 mg, preferably about 1104 mg, preferably about 1105 mg, preferably about 1106 mg, preferably about 1107 mg, preferably about 1108 mg, preferably about 1109 mg, preferably about 1110 mg, preferably about 1111 mg, preferably about 1112 mg, preferably about 1113 mg, preferably about 1114 mg, preferably about 1115 mg, preferably about 1116 mg, preferably about 1117 mg, preferably about 1118 mg, preferably about 1119 mg, preferably about 1120 mg, preferably about 1121 mg, preferably about 1122 mg, preferably about 1123 mg, preferably about 1124 mg, preferably about 1125 mg, preferably about 1126 mg, preferably about 1127 mg, preferably about 1128 mg, preferably about 1129 mg, preferably about 1130 mg, preferably about 1131 mg, preferably about 1132 mg, preferably about 1133 mg, preferably about 1134 mg, preferably about 1135 mg, preferably about 1136 mg, preferably about 1137 mg, preferably about 1138 mg, preferably about 1139 mg, preferably about 1140 mg, preferably about 1141 mg, preferably about 1142 mg, preferably about 1143 mg, preferably about 1144 mg, preferably about 1145 mg, preferably about 1146 mg, preferably about 1147 mg, preferably about 1148 mg, preferably about 1149 mg, preferably about 1150 mg, preferably about 1151 mg, preferably about 1152 mg, preferably about 1153 mg, preferably about 1154 mg, preferably about 1155 mg, preferably about 1156 mg, preferably about 1157 mg, preferably about 1158 mg, preferably about 1159 mg, preferably about 1160 mg, preferably about 1161 mg, preferably about 1162 mg, preferably about 1163 mg, preferably about 1164 mg, preferably about 1165 mg, preferably about 1166 mg, preferably about 1167 mg, preferably about 1168 mg, preferably about 1169 mg, preferably about 1170 mg, preferably about 1171 mg, preferably about 1172 mg, preferably about 1173 mg, preferably about 1174 mg, preferably about 1175 mg, preferably about 1176 mg, preferably about 1177 mg, preferably about 1178 mg, preferably about 1179 mg, preferably about 1180 mg, preferably about 1181 mg, preferably about 1182 mg, preferably about 1183 mg, preferably about 1184 mg, preferably about 1185 mg, preferably about 1186 mg, preferably about 1187 mg, preferably about 1188 mg, preferably about 1189 mg, preferably about 1190 mg, preferably about 1191 mg, preferably about 1192 mg, preferably about 1193 mg, preferably about 1194 mg, preferably about 1195 mg, preferably about 1196 mg, preferably about 1197 mg, preferably about 1198 mg, preferably about 1199 mg, preferably about 1200 mg, preferably about 1201 mg, preferably about 1202 mg, preferably about 1203 mg, preferably about 1204 mg, preferably about 1205 mg, preferably about 1206 mg, preferably about 1207 mg, preferably about 1208 mg, preferably about 1209 mg, preferably about 1210 mg, preferably about 1211 mg, preferably about 1212 mg, preferably about 1213 mg, preferably about 1214 mg, preferably about 1215 mg, preferably about 1216 mg, preferably about 1217 mg, preferably about 1218 mg, preferably about 1219 mg, preferably about 1220 mg, preferably about 1221 mg, preferably about 1222 mg, preferably about 1223 mg, preferably about 1224 mg, preferably about 1225 mg, preferably about 1226 mg, preferably about 1227 mg, preferably about 1228 mg, preferably about 1229 mg, preferably about 1230 mg, preferably about 1231 mg, preferably about 1232 mg, preferably about 1233 mg, preferably about 1234 mg, preferably about 1235 mg, preferably about 1236 mg, preferably about 1237 mg, preferably about 1238 mg, preferably about 239 mg, preferably about 1240 mg, preferably about 1241 mg, preferably about 1242 mg, preferably about 1243 mg, preferably about 1244 mg, preferably about 1245 mg, preferably about 1246 mg, preferably about 1247 mg, preferably about 1248 mg, preferably about 1249 mg, preferably about 1250 mg, preferably about 1251 mg, preferably about 1252 mg, preferably about 1253 mg, preferably about 1254 mg, preferably about 1255 mg, preferably about 1256 mg, preferably about 1257 mg, preferably about 1258 mg, preferably about 1259 mg, preferably about 1260 mg, preferably about 1261 mg, preferably about 1262 mg, preferably about 1263 mg, preferably about 1264 mg, preferably about 1265 mg, preferably about 1266 mg, preferably about 1267 mg, preferably about 1268 mg, preferably about 1269 mg, preferably about 1270 mg, preferably about 1271 mg, preferably about 1272 mg, preferably about 1273 mg, preferably about 1274 mg, preferably about 1275 mg, preferably about 1276 mg, preferably about 1277 mg, preferably about 1278 mg, preferably about 1279 mg, preferably about 1280 mg, preferably about 1281 mg, preferably about 1282 mg, preferably about 1283 mg, preferably about 1284 mg, preferably about 1285 mg, preferably about 1286 mg, preferably about 1287 mg, preferably about 1288 mg, preferably about 1289 mg, preferably about 1290 mg, preferably about 1291 mg, preferably about 1292 mg, preferably about 1293 mg, preferably about 1294 mg, preferably about 1295 mg, preferably about 1296 mg, preferably about 1297 mg, preferably about 1298 mg, preferably about 1299 mg, preferably about 1300 mg, preferably about 1301 mg, preferably about 1302 mg, preferably about 303 mg, preferably about 1304 mg, preferably about 1305 mg, preferably about 1306 mg, preferably about 1307 mg, preferably about 1308 mg, preferably about 1309 mg, preferably about 1310 mg, preferably about 1311 mg, preferably about 1312 mg, preferably about 1313 mg, preferably about 1314 mg, preferably about 1315 mg, preferably about 1316 mg, preferably about 1317 mg, preferably about 1318 mg, preferably about 1319 mg, preferably about 1320 mg, preferably about 1321 mg, preferably about 1322 mg, preferably about 1323 mg, preferably about 1324 mg, preferably about 1325 mg, preferably about 1326 mg, preferably about 1327 mg, preferably about 1328 mg, preferably about 1329 mg, preferably about 1330 mg, preferably about 1331 mg, preferably about 1332 mg, preferably about 1333 mg, preferably about 1334 mg, preferably about 1335 mg, preferably about 1336 mg, preferably about 1337 mg, preferably about 1338 mg, preferably about 1339 mg, preferably about 1340 mg, preferably about 1341 mg, preferably about 1342 mg, preferably about 1343 mg, preferably about 1344 mg, preferably about 1345 mg, preferably about 1346 mg, preferably about 1347 mg, preferably about 1348 mg, preferably about 1349 mg, preferably about 1350 mg, preferably about 1351 mg, preferably about 1352 mg, preferably about 1353 mg, preferably about 1354 mg, preferably about 1355 mg, preferably about 1356 mg, preferably about 1357 mg, preferably about 1358 mg, preferably about 1359 mg, preferably about 1360 mg, preferably about 1361 mg, preferably about 1362 mg, preferably about 1363 mg, preferably about 1364 mg, preferably about 1365 mg, preferably about 1366 mg, preferably about 1367 mg, preferably about 1368 mg, preferably about 1369 mg, preferably about 1370 mg, preferably about 1371 mg, preferably about 1372 mg, preferably about 1373 mg, preferably about 1374 mg, preferably about 1375 mg, preferably about 1376 mg, preferably about 1377 mg, preferably about 1378 mg, preferably about 1379 mg, preferably about 1380 mg, preferably about 1381 mg, preferably about 1382 mg, preferably about 1383 mg, preferably about 1384 mg, preferably about 1385 mg, preferably about 1386 mg, preferably about 1387 mg, preferably about 1388 mg, preferably about 1389 mg, preferably about 1390 mg, preferably about 1391 mg, preferably about 1392 mg, preferably about 1393 mg, preferably about 1394 mg, preferably about 1395 mg, preferably about 1396 mg, preferably about 1397 mg, preferably about 1398 mg, preferably about 1399 mg, preferably about 1400 mg, preferably about 1401 mg, preferably about 1402 mg, preferably about 1403 mg, preferably about 1404 mg, preferably about 1405 mg, preferably about 1406 mg, preferably about 1407 mg, preferably about 1408 mg, preferably about 1409 mg, preferably about 1410 mg, preferably about 1411 mg, preferably about 1412 mg, preferably about 1413 mg, preferably about 1414 mg, preferably about 1415 mg, preferably about 1416 mg, preferably about 1417 mg, preferably about 1418 mg, preferably about 1419 mg, preferably about 1420 mg, preferably about 1421 mg, preferably about 1422 mg, preferably about 1423 mg, preferably about 1424 mg, preferably about 1425 mg, preferably about 1426 mg, preferably about 1427 mg, preferably about 1428 mg, preferably about 1429 mg, preferably about 1430 mg, preferably about 1431 mg, preferably about 1432 mg, preferably about 1433 mg, preferably about 1434 mg, preferably about 1435 mg, preferably about 1436 mg, preferably about 1437 mg, preferably about 1438 mg, preferably about 1439 mg, preferably about 1440 mg, preferably about 1441 mg, preferably about 1442 mg, preferably about 1443 mg, preferably about 1444 mg, preferably about 1445 mg, preferably about 1446 mg, preferably about 1447 mg, preferably about 1448 mg, preferably about 1449 mg, preferably about 1450 mg, preferably about 1451 mg, preferably about 1452 mg, preferably about 1453 mg, preferably about 1454 mg, preferably about 1455 mg, preferably about 1456 mg, preferably about 1457 mg, preferably about 1458 mg, preferably about 1459 mg, preferably about 1460 mg, preferably about 1461 mg, preferably about 1462 mg, preferably about 1463 mg, preferably about 1464 mg, preferably about 1465 mg, preferably about 1466 mg, preferably about 1467 mg, preferably about 1468 mg, preferably about 1469 mg, preferably about 1470 mg, preferably about 1471 mg, preferably about 1472 mg, preferably about 1473 mg, preferably about 1474 mg, preferably about 1475 mg, preferably about 1476 mg, preferably about 1477 mg, preferably about 1478 mg, preferably about 1479 mg, preferably about 1480 mg, preferably about 1481 mg, preferably about 1482 mg, preferably about 1483 mg, preferably about 1484 mg, preferably about 1485 mg, preferably about 1486 mg, preferably about 1487 mg, preferably about 1488 mg, preferably about 1489 mg, preferably about 1490 mg, preferably about 1491 mg, preferably about 1492 mg, preferably about 1493 mg, preferably about 1494 mg, preferably about 1495 mg, preferably about 1496 mg, preferably about 1497 mg, preferably about 1498 mg, preferably about 1499 mg, or more preferably about or 1500 mg.

In an embodiment, the CBD or a pharmaceutically acceptable salt thereof is administered at an aforementioned dose twice per day.

In an embodiment, the CBD or a pharmaceutically acceptable salt thereof is administered at a dose of at least about 1 mg, twice per day.

In an embodiment, the CBD or a pharmaceutically acceptable salt thereof is administered at a dose of from about 1 mg to about 500 mg, twice per day.

In an embodiment, the CBD or a pharmaceutically acceptable salt thereof is administered at a dose of from about 500 mg to about 1000 mg, twice per day.

The CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof may be administered orally, topically, parenterally, transdermally, by inhalation, intranasally, by irrigation, by implant, by insufflation, topically to the eye, or aurally. The CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof may be administered in dosage unit and in formulations containing conventional non-toxic pharmaceutically acceptable carriers, adjuvants, and vehicles. Formulations include liposomal, nanoparticle, microparticle, polymer-based, dispersion, suspension, coated on a device, powder, microspheres, carrier-mediated, implant and encapsulation. In an embodiment, the CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof are administered orally. In another embodiment, the CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof are administered parenterally.

The term “parenteral” as used herein includes subcutaneous injections, aerosol for administration to lungs or nasal cavity, intravenous, intramuscular, intrathecal, intracranial, injection or infusion techniques. The present disclosure also provides suitable topical, oral and parenteral pharmaceutical formulations for use in the novel methods of treatment described herein.

In a preferred embodiment, the CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof are administered intraperitoneally.

In a preferred embodiment, the CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof are formulated for oral administration.

Suitable dosage forms for oral administration would be known to persons skilled in the art, illustrative examples of which include tablets, aqueous or oily suspensions, lozenges, troches, powders, granules, emulsions, capsules, liquids, syrups or elixirs.

In an embodiment, the CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof is in liquid, oil, tablet or capsule form.

It is further contemplated herein that the CBD or a pharmaceutically acceptable salt thereof, hydroxychloroquine or a pharmaceutically acceptable salt thereof, and compositions comprising CBD or a pharmaceutically acceptable salt thereof and/or hydroxychloroquine or a pharmaceutically acceptable salt thereof may be co-administered with one or more other agents suitable for the amelioration of symptoms associated with an inflammatory condition, such as sepsis-associated ARDS, illustrative examples of which include antibiotics, antiviral agents and antifungal agents.

In an aspect disclosed herein, there is provided a composition comprising CBD or a pharmaceutically acceptable salt thereof, and hydroxychloroquine or a pharmaceutically acceptable salt thereof.

In an embodiment, the composition comprises from about 1 mg to about 1000 mg hydroxychloroquine or a pharmaceutically acceptable salt thereof per dose. In another embodiment, the composition comprises from about 200 mg to about 400 mg hydroxychloroquine or a pharmaceutically acceptable salt thereof per dose.

In an embodiment, the composition comprises from about 1 mg to about 1500 mg CBD or a pharmaceutically acceptable salt thereof per dose. In another embodiment, the composition comprises from about 500 mg to about 1000 mg CBD or a pharmaceutically acceptable salt thereof per dose.

In another embodiment, the composition is formulated for oral administration.

In another embodiment, the composition is formulated for intraperitoneal administration.

Compositions comprising CBD and hydroxychloroquine for oral administration may contain one or more agents selected from the group of sweetening agents, flavoring agents, coloring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In an embodiment, the composition further comprises one or more pharmaceutically acceptable carriers, diluents or excipients.

Suitable pharmaceutically acceptable carriers, diluents or excipients would be known to persons skilled in the art, illustrative examples of which include inert diluents (e.g., calcium carbonate, lactose, calcium phosphate or sodium phosphate), granulating and disintegrating agents (e.g., corn starch or alginic acid), binding agents (e.g., starch, gelatin or acacia), lubricating agents (e.g., magnesium stearate, stearic acid or talc) and material to delay disintegration and absorption in the gastrointestinal tract and thereby provide a sustained action over a longer period (e.g., glyceryl monostearate or glyceryl distearate). Coating may also be performed using techniques described in the U.S. Pat. Nos. 4,256,108, 4,160,452, and 4,265,874 to form osmotic therapeutic tablets for control release.

Compositions disclosed herein may be prepared according to conventional methods well known in the pharmaceutical and nutraceutical industries, such as those described in Remington's Pharmaceutical Handbook (Mack Publishing Co., NY, USA) using suitable excipients, diluents and fillers.

Compositions suitable for oral administration may be presented as discrete units (i.e., dosage forms), each containing a predetermined amount of each component of the composition as a powder, tablet, capsule, granules, as a solution or a suspension in an aqueous liquid or non-aqueous liquid, or as an emulsion.

As described elsewhere herein, the compositions may be formulated for administration as separate unit dosage forms for administration. The unit dosage form may be suitable for a capsule, tablet, oil or liquid solution.

Oral administration of CBD has been demonstrated to be an effective administration route (reviewed by Millar et al., 2018, Frontiers in Pharmacology, 9: 1365). Similarly, oral administration of hydroxychloroquine has also been demonstrated to be effective for absorption with high bioavailability (Tett et al., 1989, British Journal of Clinical Pharmacology, 27: 771-779).

In an embodiment, the composition is for use in the treatment or prevention of an inflammatory condition.

In an embodiment, the composition is for use in the treatment or prevention of an inflammatory respiratory condition.

In an embodiment, the composition is for use in the treatment or prevention of an inflammatory respiratory condition selected from the group consisting of ARDS, COPD, asthma, bronchitis, and CF.

In an embodiment, the composition is for use in the treatment or prevention of ARDS.

In an embodiment, the composition is for use in the treatment or prevention of sepsis-associated ARDS.

In an embodiment, the composition is for use in the treatment or prevention of sepsis-associated ARDS caused by a bacterial, fungal, or viral infection, as described elsewhere herein.

In an embodiment, the composition is for use in the treatment or prevention of inflammatory bowel disease.

In an embodiment, the composition is for use in the treatment or prevention of arthritis. In another embodiment, the composition is for use in the treatment or prevention of rheumatoid arthritis.

In an aspect disclosed herein, there is provided a use of CBD or a pharmaceutically acceptable salt thereof, and hydroxychloroquine or a pharmaceutically acceptable salt thereof, in the manufacture of a medicament for the treatment or prevention of an inflammatory condition.

In an embodiment, the inflammatory condition is an inflammatory respiratory condition.

In an embodiment, the inflammatory respiratory condition is selected from the group consisting of ARDS, COPD, asthma, bronchitis and CF.

In an embodiment, the inflammatory condition is ARDS. In another embodiment, the ARDS is sepsis-associated ARDS.

In another embodiment, the inflammatory condition is inflammatory bowel disease.

In another embodiment, the inflammatory condition is arthritis. In another embodiment, the arthritis is rheumatoid arthritis.

In accordance with the methods disclosed herein, the CBD or a pharmaceutically acceptable salt thereof and hydroxychloroquine or a pharmaceutically acceptable salt thereof may be administered as a single composition or co-administered as separate compositions.

In an embodiment, the CBD or a pharmaceutically acceptable salt thereof and hydroxychloroquine or a pharmaceutically acceptable salt thereof are administered sequentially.

By “sequential” administration it is meant there is an interval between the administration of the CBD and hydroxychloroquine. The interval between sequential administrations may be seconds, minutes, hours or days. In a preferred embodiment, the interval between sequential administrations of the CBD and hydroxychloroquine is less than an hour, preferably less than 30 minutes, most preferably less than 1 minute. Sequential administration may be in any order (i.e., administration of CBD prior to the administration of the hydroxychloroquine, or administration of the hydroxychloroquine prior to the administration of CBD).

In another embodiment, the CBD or a pharmaceutically acceptable salt thereof and hydroxychloroquine or a pharmaceutically acceptable salt thereof are administered simultaneously.

By “simultaneous” administration it is meant that the CBD and hydroxychloroquine are administered at the same time. In accordance with the methods disclosed herein, the CBD and hydroxychloroquine may be administered simultaneously in a single composition or dosage form. Alternatively, the CBD and hydroxychloroquine may be administered simultaneously as two separate compositions or dosage forms.

In some embodiments, periodic re-administration of the active agents (either sequentially or co-administered) may be required to achieve a desirable therapeutic effect. The exact amounts and rates of administration of the CBD and hydroxychloroquine will depend on a number of factors, examples of which are described elsewhere herein, such as the subject's age, body weight, general health, sex and dietary requirements, as well as any drugs or agents used in combination or coincidental with the administration of the composition. Where multiple divided doses are required, these may be administered hourly, daily, weekly, monthly or at other suitable time intervals or the dose may be proportionally reduced as indicated by the exigencies of the situation.

In an embodiment, the CBD or a pharmaceutically acceptable salt thereof and hydroxychloroquine or a pharmaceutically acceptable salt thereof, or compositions comprising CBD or a pharmaceutically acceptable salt thereof and/or hydroxychloroquine or a pharmaceutically acceptable salt thereof are administered twice per day.

In an embodiment, the CBD or a pharmaceutically acceptable salt thereof and hydroxychloroquine or a pharmaceutically acceptable salt thereof, or compositions comprising CBD or a pharmaceutically acceptable salt thereof and/or hydroxychloroquine or a pharmaceutically acceptable salt thereof are administered for a period of at least one week.

In an embodiment, the CBD or a pharmaceutically acceptable salt thereof and hydroxychloroquine or a pharmaceutically acceptable salt thereof, or compositions comprising CBD or a pharmaceutically acceptable salt thereof and/or hydroxychloroquine or a pharmaceutically acceptable salt thereof are administered to the subject for a period of between 1 to 10 weeks (e.g., for 1 week, 2 weeks, 3 weeks, 4 weeks, 5 weeks, 6 weeks, 7 weeks, 8 weeks, 9 weeks, or 10 weeks).

Thus, in an embodiment, the CBD or a pharmaceutically acceptable salt thereof and hydroxychloroquine or a pharmaceutically acceptable salt thereof, or compositions comprising CBD or a pharmaceutically acceptable salt thereof and/or hydroxychloroquine or a pharmaceutically acceptable salt thereof are administered to the subject for a period of between 1 to 10 weeks, preferably for about 1 week, preferably about 2 weeks, preferably about 3 weeks, preferably about 4 weeks, preferably about 5 weeks, preferably about 6 weeks, preferably about 7 weeks, preferably about 8 weeks, preferably about 9 weeks, or more preferably for about 10 weeks.

In an embodiment, the CBD or a pharmaceutically acceptable salt thereof and hydroxychloroquine or a pharmaceutically acceptable salt thereof, or compositions comprising CBD or a pharmaceutically acceptable salt thereof and/or hydroxychloroquine or a pharmaceutically acceptable salt thereof are administered to the subject for a period of between 3 to 4 weeks.

In an embodiment, the subject is administered a loading dose of hydroxychloroquine or a pharmaceutically acceptable salt thereof on the first day of treatment followed by a maintenance dose for each day thereafter, wherein the loading dose is from about 1 mg to about 1000 mg, twice per day, and wherein the maintenance dose is from about 1 mg to about 1000 mg, twice per day. In an embodiment, the subject is administered a loading dose of hydroxychloroquine or a pharmaceutically acceptable salt thereof on the first day of treatment followed by a maintenance dose for each day thereafter, wherein the loading dose is from about 1 mg to about 500 mg, twice per day, and wherein the maintenance dose is from about 1 mg to about 500 mg, twice per day. In an embodiment, the subject is administered a loading dose of hydroxychloroquine or a pharmaceutically acceptable salt thereof on the first day of treatment followed by a maintenance dose for each day thereafter, wherein the loading dose is from about 1 mg, twice per day, and wherein the maintenance dose is from about 1 mg, twice per day.

In an embodiment, the subject is administered from about 1 mg to about 1500 mg CBD or a pharmaceutically acceptable salt thereof with the loading dose of hydroxychloroquine or a pharmaceutically acceptable salt thereof, twice per day. In another embodiment, the subject is administered from about 1 mg to about 1000 mg CBD or a pharmaceutically acceptable salt thereof with the loading dose of hydroxychloroquine or a pharmaceutically acceptable salt thereof, twice per day. In an embodiment, the subject is administered from about 1 mg to about 1500 mg CBD or a pharmaceutically acceptable salt thereof with the maintenance dose of hydroxychloroquine or a pharmaceutically acceptable salt thereof, twice per day. In another embodiment, the subject is administered from about 500 mg to about 1000 mg CBD or a pharmaceutically acceptable salt thereof with the maintenance dose of hydroxychloroquine or a pharmaceutically acceptable salt thereof, twice per day.

The term “loading dose” as used herein refers to an initial higher dose of a drug that is to be given at the commencement of a course of treatment before dropping down to a lower maintenance dose.

The term “maintenance dose” as used herein refers to the maintenance rate (mg/h) of a drug following administration, which is equal to the rate of elimination at steady state.

In other embodiments disclosed herein, the CBD or a pharmaceutically acceptable salt thereof and hydroxychloroquine or a pharmaceutically acceptable salt thereof are formulated as separate unit dosage forms for administration. The unit dosage form may be suitable for oral solution, capsule or tablet form. Those skilled in the art will appreciate that unit dosage forms comprising CBD or a pharmaceutically acceptable salt thereof and/or hydroxychloroquine or a pharmaceutically acceptable salt thereof need not be of the same type. Thus, methods of the present disclosure contemplate the administration of, for example, CBD in a liquid form and the hydroxychloroquine as a capsule or tablet.

### Kits

In an aspect disclosed herein, there is provided a kit comprising CBD or a pharmaceutically acceptable salt thereof, and hydroxychloroquine or a pharmaceutically acceptable salt thereof, for use in the treatment or prevention of an inflammatory condition.

In an embodiment, the kit comprises the CBD or a pharmaceutically acceptable salt thereof and hydroxychloroquine or a pharmaceutically acceptable salt thereof in separate unit dosage forms for the loading dose and maintenance dose, as described elsewhere herein.

### Methods for Modulating an Immune Response

In another aspect disclosed herein, there is provided a method of modulating an immune response comprising administering to a subject in need thereof cannabidiol (CBD) or a pharmaceutically acceptable salt thereof, and hydroxychloroquine or a pharmaceutically acceptable salt thereof.

Persons of ordinary skill in the art would appreciate that modulation of the immune response in accordance with the methods disclosed herein can be determined by a variety of methods known in the art, illustrative examples of which include measuring changes in cytokine production (e.g., levels, concentrations, ratios) in the subject such as before and after treatment or during the course of treatment. The term “modulation”, as used herein, is to be understood to mean a reduction or an increase in the immune response, as determined, for example, by a decrease or increase in the level, concentration and/or ratio of inflammatory mediators, such as cytokines.

The terms “level” and “amount” are used interchangeably herein to refer to a quantitative amount (e.g., moles or number), a semi-quantitative amount, a relative amount (e.g., weight %, or mole % within a class, or a ratio), a concentration, and the like. Thus, these terms encompass absolute or relative amounts or concentrations, including of inflammatory mediators, in a sample.

Inflammatory mediators, such as cytokines, may be quantified or detected using any suitable technique, including, but not limited to, nucleic acid- and protein-based assays. In illustrative nucleic acid-based assays, nucleic acid is isolated from cells contained in a biological sample according to standard methodologies (Sambrook, et al., 1989, Molecular Cloning: A Laboratory Manual; and Ausubel et al., 1994, Current Protocols in Molecular Biology).

In other illustrative embodiments, protein levels of inflammatory mediators can be measured using protein-based assays known in the art. For example, an antibody-based technique may be employed to determine the level of an autoantibody in a sample, illustrative examples of which include immunoassays, such as the enzyme-linked immunosorbent assay (ELISA), immunohistochemistry (IHC) and the radioimmunoassay (RIA).

In an embodiment, protein expression is measured using a multiplexed protein expression analysis method. In another embodiment, the multiplexed protein expression analysis method is a protein microarray or Luminex bead array.

Protein-capture arrays that permit simultaneous detection and/or quantification of a large number of proteins may also be employed. For example, low-density protein arrays on filter membranes, such as the universal protein array system allow imaging of arrayed antigens using standard ELISA techniques and a scanning charge-coupled device (CCD) detector. Exemplary protein capture arrays include protein function arrays comprising spatially addressed protein-binding molecules (i.e., antigens), which can facilitate extensive parallel analysis of autoantibodies with specificity for the antigens that comprise the protein function array. Central to this type of analysis is the retention of the correctly folded protein confirmation of the arrayed antigen. Protein function arrays have been shown to have the required properties of specificity and acceptable background, and are available commercially (e.g., Sengenics). Various methods for the preparation of protein function arrays have been reported (see, e.g., Gnjatic et al., 2009, Journal of Immunological Methods, 341(50): 1-2; PCT/GB01/00395, PCT/GB02/05499, PCT/GB03/00362). Individual spatially distinct functional proteins are typically attached to a support surface, which is generally planar or contoured. Common physical supports include glass slides, silicon, microwells, nitrocellulose or PVDF membranes, and magnetic and other microbeads.

Particles in suspension can also be used as the basis of arrays, providing they are coded for identification; systems include colour coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents (e.g., antibodies to inflammatory mediators or inflammatory mediator-binding fragments thereof) are typically attached to an individual particle to provide the spatial definition or separation of the array. The particles may then be assayed separately, but in parallel, in a compartmentalised way, for example in the wells of a microtiter plate or in separate test tubes.

In an illustrative example, a patient or control sample is delivered to a protein function array under conditions suitable for protein or peptide binding, and the array is washed to remove unbound or non-specifically bound components of the sample from the array. Next, the array is incubated with fluorescently-labelled antibody to detect the interaction between array antigens and inflammatory mediator(s) present in the sample. The presence or amount of protein or peptide bound to each feature of the array is detected using a suitable fluorescence detection system. The amount of protein bound to a feature of the array is proportional to the intensity of fluorescence. In certain embodiments, local background fluorescence obtained from control features of the array are automatically subtracted and relative fluorescent units (rfu) for each feature of the array is recorded.

In some embodiments, the protein function array is a Luminex-based multiplex assay, which is a bead-based multiplexing assay, where beads are internally dyed with fluorescent dyes to produce a specific spectral address. Biomolecules (such as an oligo or antibody) can be conjugated to the surface of beads to capture analytes of interest; that is, inflammatory markers, e.g., cytokines. Flow cytometric or other suitable imaging technologies known to persons skilled in the art can then be used for characterisation of the beads, as well as for detection of analyte presence. The Luminex technology enables are large number of proteins, genes or other gene expression products (e.g., 100 or more, 200 or more, 300 or more, 400 or more) to be detected using very small sample volume (e.g., in a 96 or 384-well plate).

In some embodiments, the level of an inflammatory mediator can be normalised against a housekeeping biomarker. The term “housekeeping biomarker” refers to a biomarker or group of biomarkers (e.g., polynucleotides and/or polypeptides), which are typically found at a constant level in the cell type(s) or tissue(s) being analysed and across the conditions being assessed.

In other embodiments, the level of an inflammatory mediator measured using a protein array can be normalised by both intra- and inter-array data normalisation. For example, the overall median value of all median relative fluorescent units (rfu) of each protein in a protein function array (excluding data from control proteins) is calculated and intra-array normalisation achieved by dividing the median of the quadruplicate spots of each protein on the array, by the overall median value of all the proteins on the array in each sample. Inter-array normalisation can be achieved using bioinformatics software packages that are known in the art. For example, inter-array normalisation can be achieved using the normalize. quantiles package in R (Bolstad et al., 2003, Bioinformatics, 19(2): 185-193).

It would be understood by those skilled in the art, as described elsewhere herein, that the method of analysing the level of an inflammatory mediator in a sample can be quantitative, semi-quantitative or qualitative in nature. For example, quantitative analyses will typically provide a concentration or number of an inflammatory mediator nucleic acid molecule or protein in the sample within an appropriate error margin (e.g., mean+/−standard deviation). By contrast, semi-quantitative or qualitative analyses will typically provide an indication of the relative amount of an inflammatory mediator in a sample. This may involve a comparison of an amount of an inflammatory mediator in a first sample with an amount of an inflammatory mediator in a second sample and making a determination as to the relative amount of the inflammatory mediator between the first and second samples.

It will be understood by persons skilled in the art that, where a comparison is made to a reference value, then the manner in which the sample is assessed for the level of the one or more inflammatory mediators should be substantially identical to the manner in which the reference value is derived in order to ensure that an appropriate comparison can be made for the purposes of determining whether or not the immune response has been modulated in a subject following administration of CBD and hydroxychloroquine

In an embodiment, the method of modulating an immune response comprises decreasing the level of an inflammatory mediator relative to a reference level.

In an embodiment, the inflammatory mediator is a cytokine.

The term “cytokine” as used herein refer to factors that exert a variety of effects on cells, for example, inducing growth or proliferation, illustrative examples of which include IL-1β, IL-2, IL-10, IL-12 (p70), IFN-γ, TNF-α, IL-1α, IL-3, IL-4, IL-5, IL-6, IL-9, IL-12(p40), IL-13, IL-17A, Eotaxin, G-CSF, GM-CSF, IFN-γ, KC, MCP-1(MCAF), MIP-1α, MIP-1β, RANTES and CXCL-1.

In an embodiment, the method of modulating an immune response comprises decreasing the level of an inflammatory cytokine selected from the group consisting of IL-1β, IL-6, TNF-α, IL-1α, IL-12(p70), IFN-γ, CXCL-1, MCP-1 and MIP-1α, relative to a reference level.

In an embodiment, the method of modulating an immune response comprises decreasing the level of an inflammatory cytokine selected from the group consisting of IL-1β, IL-6 and TNF-α relative to a reference level.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### EXAMPLES

### Materials

### Active Agents

Cannabidiol (CBD) and hydroxychloroquine (HCQ), were purchased by Pharmacology Discovery Services Taiwan, Ltd. The formulations are summarised in Table 3.

TABLE 3
Formulations for in vivo analysis

| Test | | | | Light | | |
|---|---|---|---|---|---|---|
| Compound | Vehicle | Solubil. ^{(a)} | Color | Protect^{(b)} | Temp.^{(c)} | mg/mL |
| CBD | Ethanol:Tween20:saline | I | White | Y | 4° C. | 10 |
| | (1:1:18) | | | | | |
| CBD | Ethanol:Tween20:saline | I | Light | Y | 4° C. | 5 and |
| | (1:1:18) | | white | | | 2.5 |
| CBD | Ethanol:Tween20:saline | I | Pale | Y | 4° C. | 1 |
| | (1:1:18) | | white | | | |
| CBD | Ethanol:Tween20:saline | S | Colorless | Y | 4° C. | 0.5 and |
| | (1:1:18) | | | | | 0.1 |
| CBD | Sesame oil | S | Pale | Y | 4° C. | 0.1 and |
| | | | yellow | | | 1 |
| HCQ | 0.9% saline | S | Colorless | Y | RT | 10, 5, 2.5, |
| | | | | | | 1, 0.5, and |
| | | | | | | 0.1 |
| HCQ | PBS | S | Colorless | Y | RT | 0.2 and |
| | | | | | | 2 |
| Dexamethasone | 1% Tween80 | I | Light | N | 4° C. | 1 |
| | | | white | | | |

^{(a)} This is based on a visual observation. S: soluble; I: insoluble (suspension or precipitation).
^{(b)}Y: formula is kept in tube or vial with brown color or covered with aluminum foil; N: no protection from light.
^{(c)}RT: prepared fresh and stored between 20-25° C.; 4° C.: prepared fresh and stored in the refrigerator or kept on ice.

### Animals

Male C57BL/6 or BALB/c mice weighing 19±1 g and female Lewis rats were provided by BioLasco Taiwan. Animals were acclimated for 3 days prior to use and were confirmed to be in good health. All animals were maintained in a hygienic environment with controlled temperature (20-24° C.), humidity (30%-70%) and 12 hours light/dark cycles. Free access to sterilised standard lab diet (MFG, Oriental Yeast Co., Ltd., Japan) and autoclaved tap water was provided. All aspects of the work, including housing, experimentation, and disposal of animals was performed in general accordance with the Guide for the Care and Use of Laboratory Animals: Eighth Edition (National Academy Press, Washington, D.C., 2011) in our AAALAC-accredited laboratory animal facility. The animal care and use protocol was reviewed and approved by the IACUC at Pharmacology Discovery Services Taiwan, Ltd.

### Chemicals

0.9% NaCl (Sintong Chemical Industry Co., Ltd. Taiwan), Bio-Plex mouse cytokine Th1-7plex panel (Bio-Rad, USA), Bio-Plex mouse cytokine group I 23-plex panel (Bio-Rad, USA), Lipopolysaccharide (Escherichia coli 055:B5, Sigma L-2880, USA), Mouse D-Dimer (D2D) ELISA kit (MyBioSource, USA), Phosphate buffered saline (Sigma, USA) and Water for injection (WFI) (Tai-Yu, Taiwan).

### Methods

### Lipopolysaccharide (LPS)-Induced Sepsis

Male C57BL/6 mice weighing 10-20 g were used. Vehicle and active agents (i.e., CBD and hydroxychloroquine) alone or in combination were administered intraperitoneally (IP) (Table 4) or orally (Table 5) at 1 hour before LPS (100 μg/100 μL/mouse) injected intravenously (IV). At 2 hours after LPS injection, blood was collected from all mice by cardiac puncture, sera processed and assayed for biomarkers using either the Th17-plex for IL-1β, IL-2, IL-10, IL-12 (p70), IFN-γ and TNF-α by Luminex, plus IL-6 and D-dimer by ELISA, or the 23-plex for IL-1α, IL-1β, IL-2, IL-3, IL-4, IL-5, IL-6, IL-9, IL-10, IL-12(p40), IL-12(p70), IL-13, IL-17A, Eotaxin, G-CSF, GM-CSF, IFN-γ, KC, MCP-1(MCAF), MIP-1α, MIP-1β, RANTES, and TNF-α by Luminex, plus IL-6 by ELISA. ANOVA followed by Dunnett's test was applied for comparison between vehicle and treatment groups. p<0.05 is considered significant.

For synergy analysis, data were baseline subtracted using the cytokine levels of sham treated mice (i.e., no LPS injection), before normalising the values for each cytokine relative to maximum values across the groups. The normalised values were used to calculate the relative inhibition where a value of 1 represents complete inhibition and a value of 0 represents no inhibition. Synergy was calculated using the statistical methods described elsewhere herein.

### LPS-Induced Pulmonary Inflammation

Male C57BL/B6 mice weighing 10-20 g were used. Vehicle and active agents (i.e., CBD and hydroxychloroquine) alone or in combination were administered intraperitoneally (IP) at 1 hour before LPS (˜80 mg/kg) was administered intratracheally. 24 hours after LPS injection, mice were anaesthetized with pentoparbital and 0.5 mL of PBS was administered twice through a tracheal cannula after which approximately 0.6 mL of broncheoalveolar lavage fluid (BALF) was obtained. The BALF was assayed for IL-1β, TNF-α, MCP-1(MCAF) and CXCL-1 by Luminex. ANOVA followed by Dunnett's test was applied for comparison between vehicle and treatment groups. p<0.05 was considered significant.

In a separate test group of animals with pulmonary inflammation induced as detailed above, lungs were harvested at termination (five lobes per lung) and fixed in 10% neutral buffered formalin for histopathology. Samples were sectioned at 4-6 μm and stained with hematoxylin and eosin (H&E) to examine inflammatory lesions as described by Shackleford et al. (2002, Toxicologic Pathology, 30(1): 93-96). Briefly, a score of 0 corresponds to not present, and a score of 5 indicates a severe/high number of lesions. Scores were averaged across lobes for each animal and then across animals for each treatment group.

For synergy analysis, data were baseline subtracted using the cytokine levels of sham treated mice (i.e., no LPS injection), before normalising the values for each cytokine relative to maximum values across the groups. The normalised values were used to calculate the relative inhibition where a value of 1 represents complete inhibition and a value of 0 represents no inhibition. Synergy was calculated using the statistical methods described elsewhere herein.

### 2,4,6-Trinitrobenzene Sulfonic Acid (TNBS)-Induced Colitis

Male BALB/c were used as an in vivo model of colitis, induced by intracolonic administration of 2,4,6-trinitrobenzene sulfonic acid (TNBS) in 50% ethanol, as previously described by Antoniou et al. (2016, Annals of Medicine and Surgery, 11: 9-15). Briefly, the ethanol disrupts the intestinal barrier, permitting TNBS to interact with colon proteins. Interaction of TNBS with high-molecular weight proteins renders them immunogenic, leading to Th1 mediated inflammation, which is causative of symptoms include inconsistent stool formation and blood in the faeces.

Vehicle and active agents (i.e., CBD and hydroxychloroquine) alone or in combination were administered intraperitoneally (IP) once a day starting from Day 1 (i.e., 24 hours before TNBS) to Day 4, for a total of 4 consecutive days (Days 1-4). On the Day of TNBS challenge (i.e., Day 2), test articles and vehicle were given 2 hrs before TNBS.

Colon tissues were harvested in all animals at termination. Colon samples were taken at 0.5, 2, and 3.5 cm from the anus, fixed in formalin, and then embedded in paraffin blocks. Four-micrometer tissue sections were cut and stained with H&E for histological analysis in accordance with the method of Dieleman et al. (1998, Clinical Experimental Immunology, 114: 385-391). Histological criteria included: abnormalities of mucosal architecture, extent of inflammation, erosion or ulceration, epithelial regeneration, and the percentage involvement by the disease process. The scoring was based on the findings of the observers by examining three sections from each colon per animal. Total score for colitis (Total Colitis Index) were added, resulting in a combined histological score range from 0 to 60.

Other endpoint measures of stool consistency score (i.e., 0—normal stools, 1, soft but still formed stools, 2—very soft stools, 3—diarrhoea), faecal occult blood score (i.e., 0-negative hemoccult, 1—positive hemoccult, 2—blood traces in stool visible, 3—rectal bleeding), colon weight, colon macroscopic damage score (a composite of adhesions, strictures, ulcers/inflammations and wall thickness) and myeloperoxidase levels in the colon tissue were also assessed. The data from each of these endpoints was baseline subtracted using the equivalent measures from sham treated mice (i.e., no TNBS) and reduction in any measure calculated as a relative reduction. Synergy was calculated using the statistical methods described elsewhere herein.

### Collagen-Induced Arthritis

Female Lewis rats were challenged with porcine type-II collagen with Freund's adjuvant on Day 1 (0.2 mg/0.2 mL/rat) by subcutaneous injection at the base of the tail to induce arthritis. A booster injection at 0.1 mg/0.1 mL/rat) was administered on Day 7. On Day 16, rats were allocated into groups of six based on hind paw volume, with animals allocated into groups to provide a similar distribution of hind paw volumes.

Vehicle and active agents (i.e., CBD and hydroxychloroquine) alone or in combination were administered intraperitoneally (IP) once a day starting from Day 17 to Day 30, i.e., for a total of 14 consecutive days.

Disease was assessed by measuring hind paw volume with plethysmometer and using a qualitative severity score system as outlined in Table 32 on Day 1, 7, 10, 14, 16, 18, 20, 22, 24, 26, 28 and 30.

Post-termination on Day 30, blood was collected from all rats and analysed for levels of the inflammatory cytokines IL-1β, IL-6 and TNF-α by ELISA. Both hind paws were harvested, weighed and formalin-fixed for histopathology. Tissue was assessed to evaluate cartilage and bone destruction by pannus formation and mononuclear cell infiltration in synovial tissues according to the scoring matrices set out in Tables 33 and 34. A total histology score, being the sum of the pannus formation and mononuclear cell infiltration score, was also calculated.

For synergy analysis, data were baseline subtracted using sham treated mice (i.e., no collagen injection), before normalising the values relative to vehicle control. Synergy was calculated using the statistical methods described elsewhere herein.

### In Vitro Analysis of the Anti-Inflammatory Activity of the Combination of CBD and Hydroxychloroquine

The anti-inflammatory activity the combination of CBD and hydroxychloroquine was assessed by determining cytokine release from human peripheral blood mononuclear cells (PBMCs) stimulated with bacterial lipopolysaccharide (LPS) using a Luminex based assay. A 96-well microtitre plate-based checkerboard assay with seven concentrations (including the no drug control) of CBD and hydroxychloroquine were assessed in combination was used to determine the drug-drug interaction. Briefly, frozen PMBCs from two independent donors were thawed, diluted in culture medium, seeded into 96-well microtitre plates and incubated at 37° C., 5% CO₂ for 1 hour prior to the addition of test compounds. After the addition of the test compounds the cells were returned to the incubator for 1 hour, after which LPS was added to the wells. Plates were incubated at 37° C., 5% CO₂ for 24 hours. After 24 hours, cell culture supernatants were removed and analysed for cytokine levels using Luminex methodology according to manufacturers' instructions. All values had the vehicle background subtracted prior to further analysis. Three plates were set up in parallel using the PBMCs from the same donor. Inhibition of cytokine release was determined relative to the no treatment control (i.e., that was stimulated with LPS) and averaged across the three replicates. A cytotoxicity assay using AlmarBlue with the identical plate set up was performed in parallel.

### Statistical Analysis

Drug synergy was determined using the Bliss Independence method where the predicted effect of the drug combination is calculated using the equation:

E_{pred A+B}=(E_{A}+E_{B})−(E_{A}E_{B})

Synergy values for each combination of drug concentrations were calculated using the Excess Over Bliss method (the difference between the observed and predicted inhibition) described by, e.g., Liu et al. (2018, Statistics in Biopharmaceutical Research, 10: 112-122, where a value greater than 0 is indicative of synergy and the higher the value the stronger the synergy.

### Dose Conversion

Doses of CBD and hydroxychloroquine used in in vivo models of the inflammatory conditions exemplified herein may be converted (i.e., theoretically extrapolated) to an appropriate human starting dose using methods known in the art, illustrative examples of which include the U.S. Department of Health and Human Services, Food and Drug Administration (FDA) Guidance for Industry—Estimating the Maximum Safe Starting Dose in Initial Clinical Trials for Therapeutics in Adult Healthy Volunteers (2005, available at http://www.fda.gov/72309/download). The derived starting dose can be tested for safety and efficacy using routine methods, such as first-in-human clinical trials for new therapeutic modalities in adult healthy volunteers.

### Example 1—Exemplary Composition for the Treatment of Sepsis-Associated ARDS

Exemplary loading dose compositions according to the present disclosure comprise the following ingredients:

| Active Agent | Dose Range |
|---|---|
| CBD | 500 mg-1000 mg |
| Hydroxychloroquine | 400 mg |

Exemplary maintenance dose compositions according to the present disclosure comprise the following ingredients:

| Active Agent | Dose Range |
|---|---|
| CBD | 500 mg-1000 mg |
| Hydroxychloroquine | 200 mg |

Both the loading dose and maintenance dose compositions according to the present disclosure are formulated for oral administration according to a twice daily dosage regimen.

### Example 2—Exemplary Methods for the In Vitro Treatment of Sepsis-Associated ARDS

The in vitro treatment of ARDS according to the present disclosure will be assessed using classic rodent model systems of sepsis-associated ARDS (e.g., as reviewed by Matute-Bello et al., 2008, American Journal of Physiology—Lung Cellular and Molecular Physiology, 295: L379-L399) to evaluate the pharmacodynamics of CBD and hydroxychloroquine, or compositions comprising CBD and/or hydroxychloroquine. Plasma concentration of the active agents, reduction in markers of acute inflammation and increased pulmonary function are the key experimental endpoints for these methods.

### Example 3—Exemplary Methods for the Treatment of Sepsis-Associated ARDS

An exemplary method for the treatment of sepsis-associated ARDS according to the present disclosure is as follows:
Composition of Example 1: Loading dose—oral administration or co-administration of active agents to the subject twice per day on the first day of treatment.
   Maintenance dose—oral administration or co-administration of active agents to the subject twice per day from the second day of treatment.

The duration of therapy is acute, expected to be between 3-4 weeks.

Subjects are randomized into four groups to assess the efficacy and safety of CBD and hydroxychloroquine, or compositions comprising CBD and/or hydroxychloroquine for the treatment of sepsis-associated ARDS as follows:
Group 1: Loading dose: 500 mg-1000 mg CBD+400 mg hydroxychloroquine, twice per day.
   Maintenance dose: 500 mg-1000 mg CBD+200 mg hydroxychloroquine, twice per day.
Group 2: Loading dose: 500 mg-1000 mg CBD+placebo, twice per day.
   Maintenance dose: 500 mg-1000 mg CBD+placebo, twice per day.
Group 3: Loading dose: Placebo+400 mg hydroxychloroquine, twice per day.
   Maintenance dose: Placebo+200 mg hydroxychloroquine, twice per day.
Group 4: Placebo+placebo

Efficacy of each trial group is assessed based on a target efficacy for clinical significance (i.e., reduction in markers of acute inflammation/reduction of LIS>20%).

ARDS is an inflammatory disorder associated with the release of pro-inflammatory cytokines, together with accumulation of immune cells within the pulmonary tissues. The production of pro-inflammatory transcription factors (e.g., NF-κβ) by such immune cells are key drivers of pathogenesis. It is expected that the administration of CBD and hydroxychloroquine will simultaneously reduce inflammation and promote immune system regulatory pathways that may reverse pulmonary tissue damage in patients with ARDS to effectively treat ARDS and ameliorate associated symptoms. Without being bound by theory or by a particular mode of application, the anti-inflammatory activity of CBD likely synergises with the anti-inflammatory activity and promotion of the resolution of inflammation mediated by the hydroxychloroquine to reduce the acute pulmonary inflammatory response to treat ARDS.

### Example 4—Intraperitoneal Administration of CBD or Hydroxychloroquine Modulates the Immune Response to LPS-Induced Sepsis In Vivo

CBD and hydroxychloroquine were evaluated for their ability to modulate the immune response to LPS-induced sepsis in mice at five different dose levels following intraperitoneal administration.

Administration of 1, 5, 10, 25, 50 and 100 mg/kg CBD showed a dose-dependent increase on the LPS-induced D-dimer, IL-1β and IL-10 levels compared to vehicle control. Moreover, when compared to the vehicle group, CBD at 100 mg/kg IP showed significant (p<0.05) increase on IL-1β and IL-10 levels in the study (Table 8).

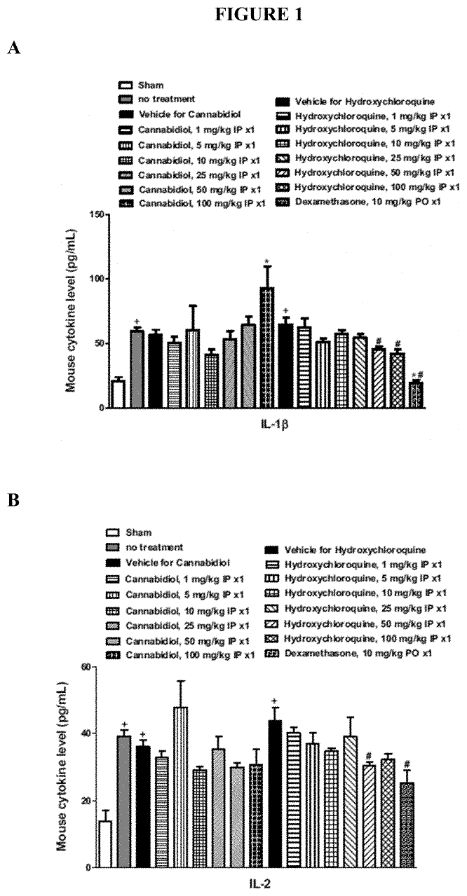

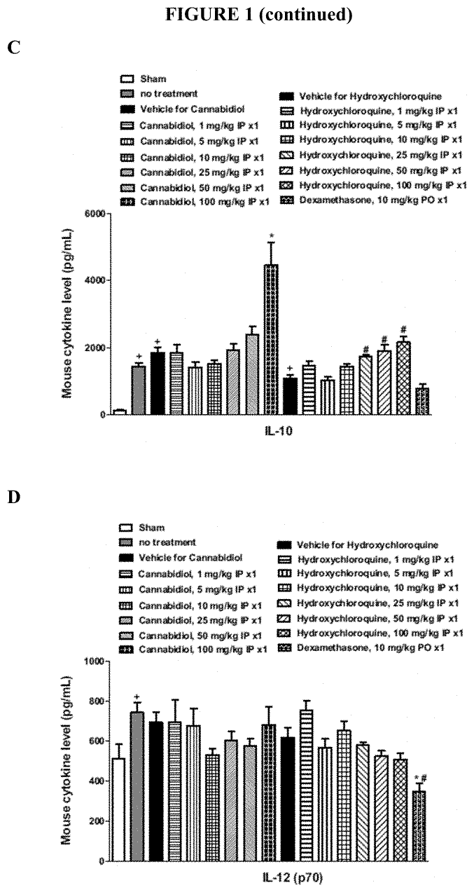

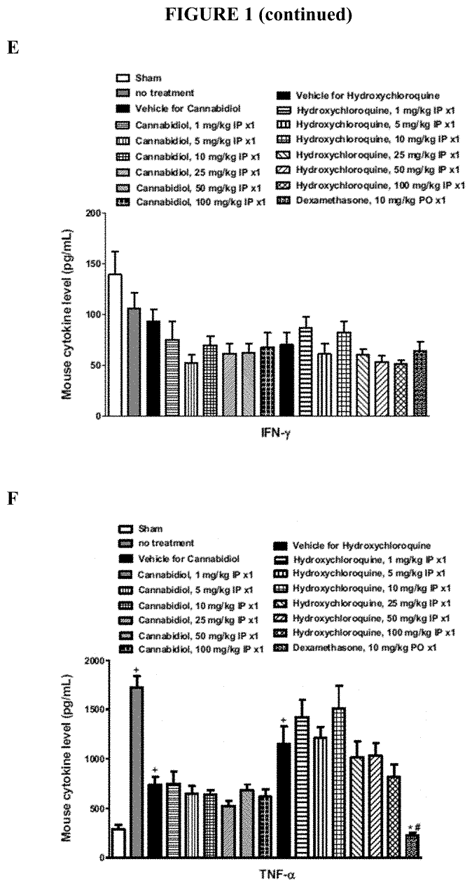

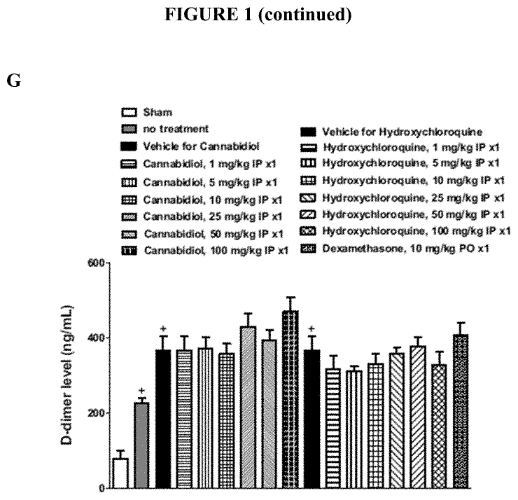

A moderate reduction in levels of IL-1β, IL-2, IL-12(p70), IFN-γ and TNF-α was observed at the 10 mg/kg dose of CBD compared to the vehicle group (Table 7). Moderate reductions in IFN-γ and TNF-α or IFN-γ alone were also observed at 25 and 5 mg/kg CBD doses, respectively (FIG. 1, Table 7). The reduction in cytokine levels at intermediate doses, but not low or high doses, is indicative of a bell-shaped dose response curve for CBD in the reduction of LPS-induced cytokine levels.

Administration of 1, 5, 10, 25, 50 and 100 mg/kg hydroxychloroquine also showed a dose-dependent increase on D-dimer level and significant (p<0.05) increase on IL-10 levels compared to the vehicle group. Moreover, when compared to the vehicle group, hydroxychloroquine given at 50 and 100 mg/kg produced significant (p<0.05) decreases in IL-1β and IL-2 levels and a moderate decrease on IL-12(p70), TNF-α and IFN-γ levels in the in vivo model of LPS-induced sepsis (FIG. 1, Table 9).

As expected, the positive control, dexamethasone at 10 mg/kg PO significantly (p<0.05) reduced the levels of levels of IL-1β, IL-2, IL-12(p70) and TNF-α compared to the vehicle group in the study (FIG. 1, Table 10).

Taken together, these data demonstrate that the anti-inflammatory dose response curves for CBD and hydroxychloroquine are likely acting via different mechanisms. Drugs with different methods of action are optimal candidates for the design of synergistic combination drugs that perform better overall that their constituent parts.

### Example 5—Oral Administration of a Combination of CBD and Hydroxychloroquine Modulates the Immune Response to LPS-Induced Sepsis In Vivo

CBD and hydroxychloroquine, alone and in combination, were evaluated for their ability to modulate the immune response to LPS-induced sepsis in mice at two different dose levels following oral administration.

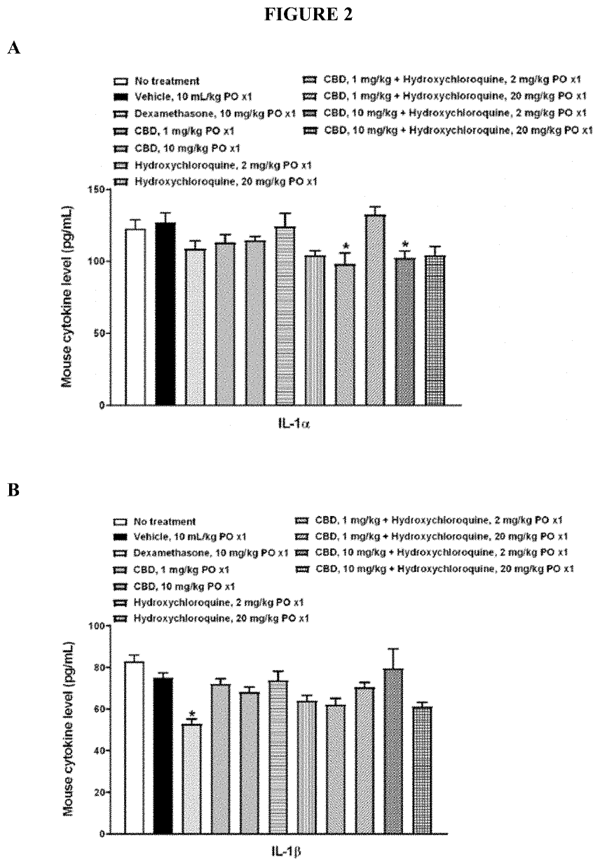

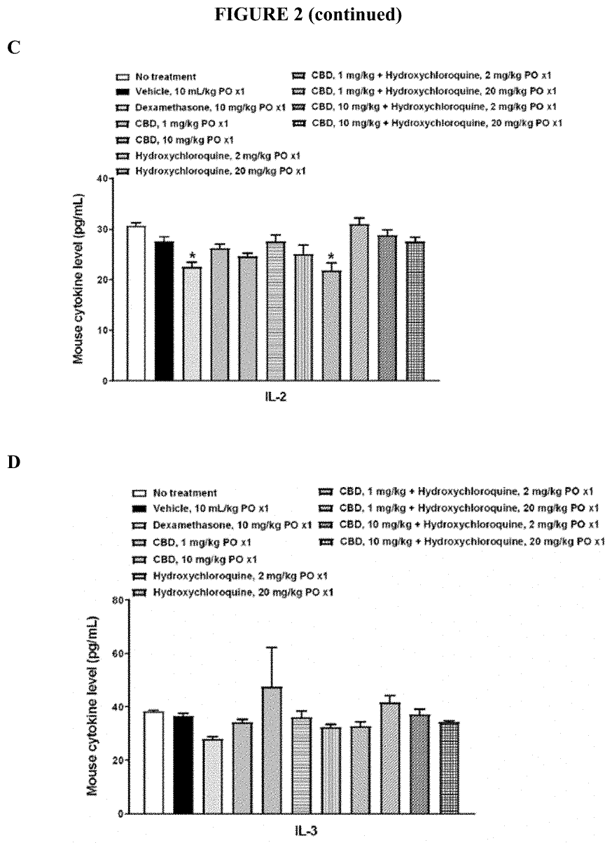

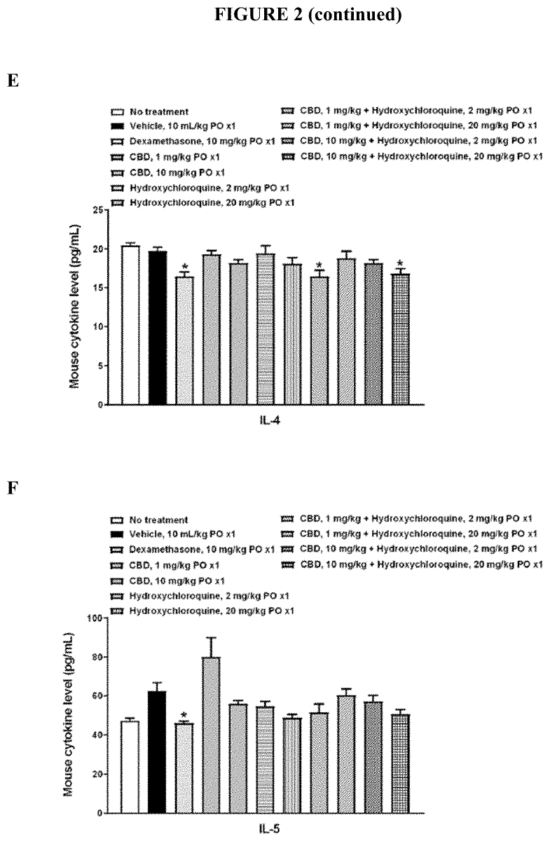

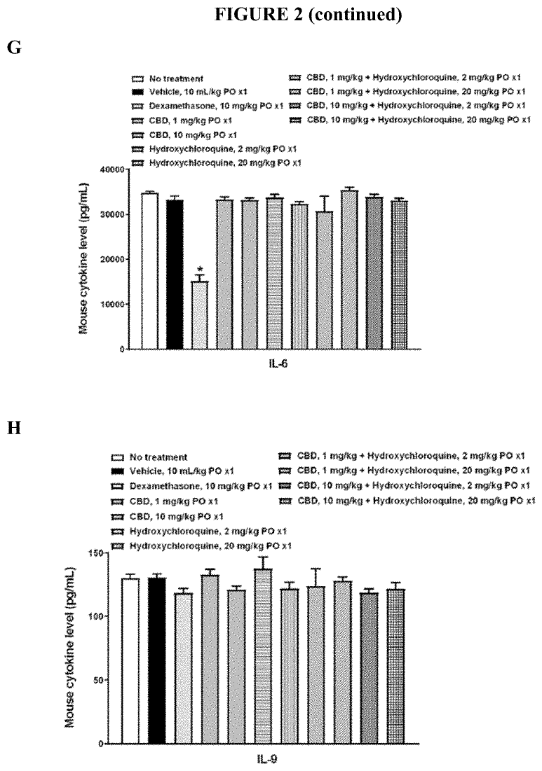

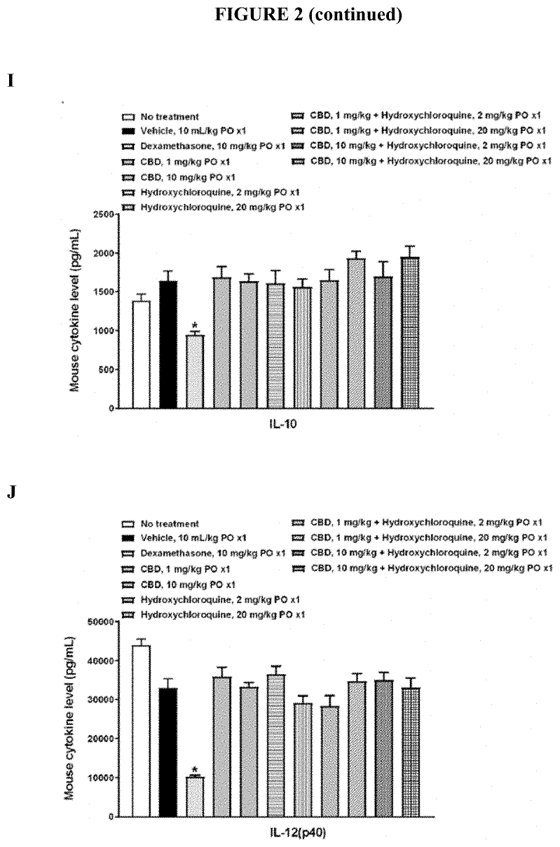

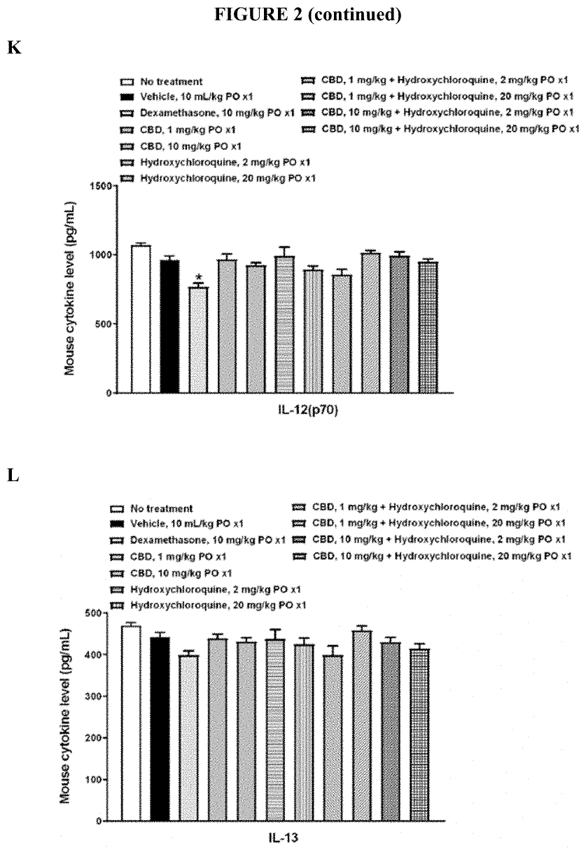

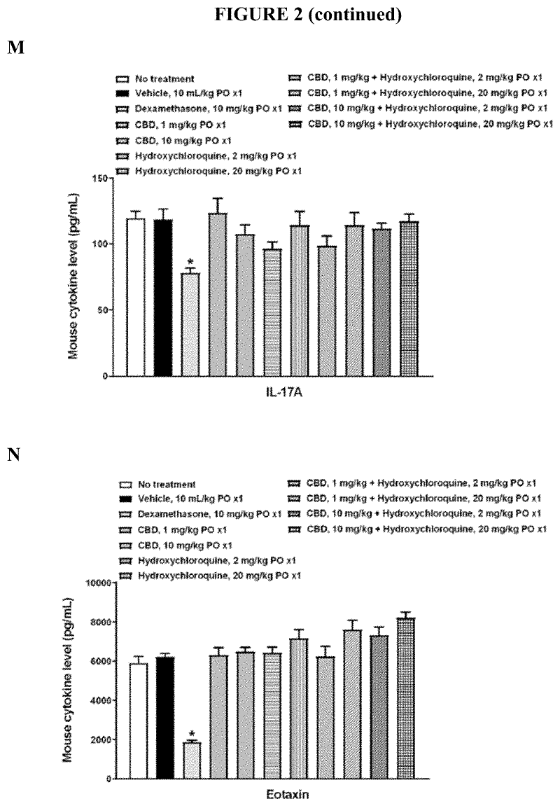

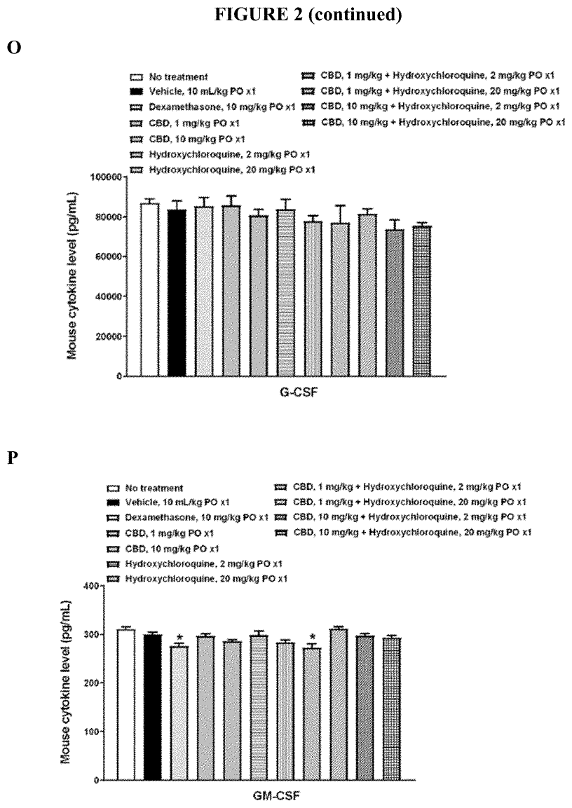

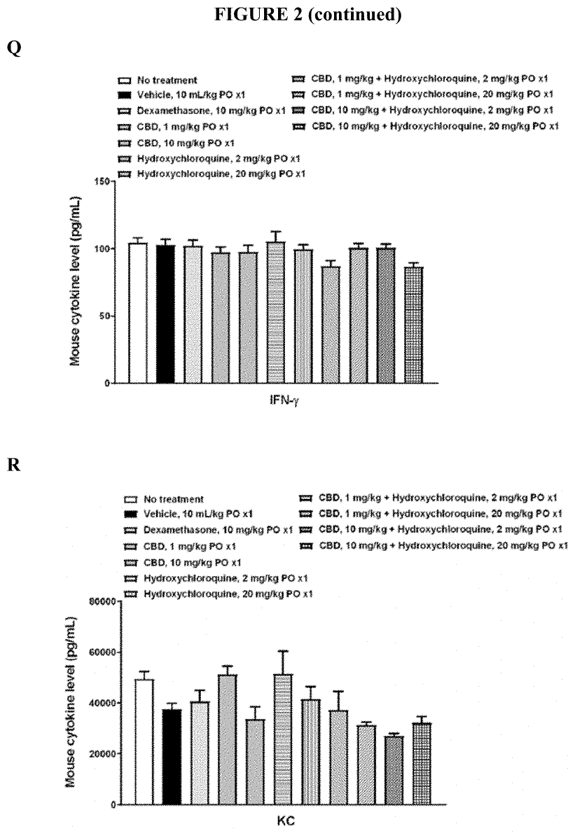

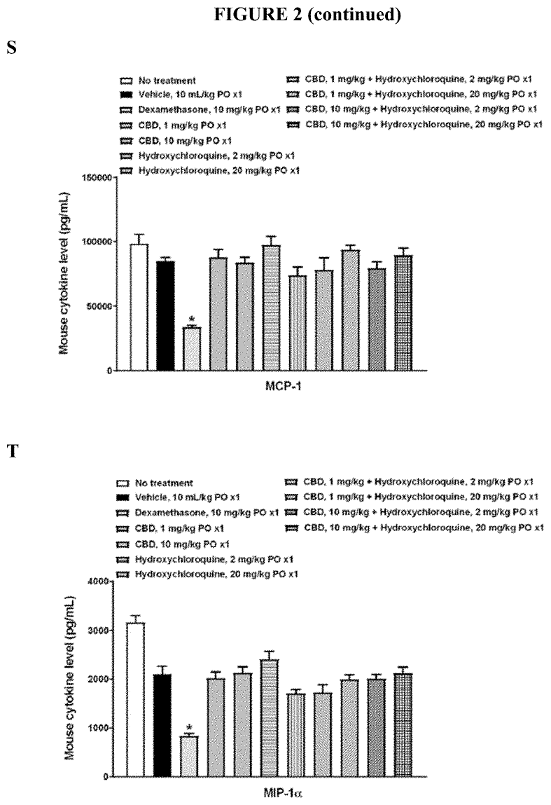

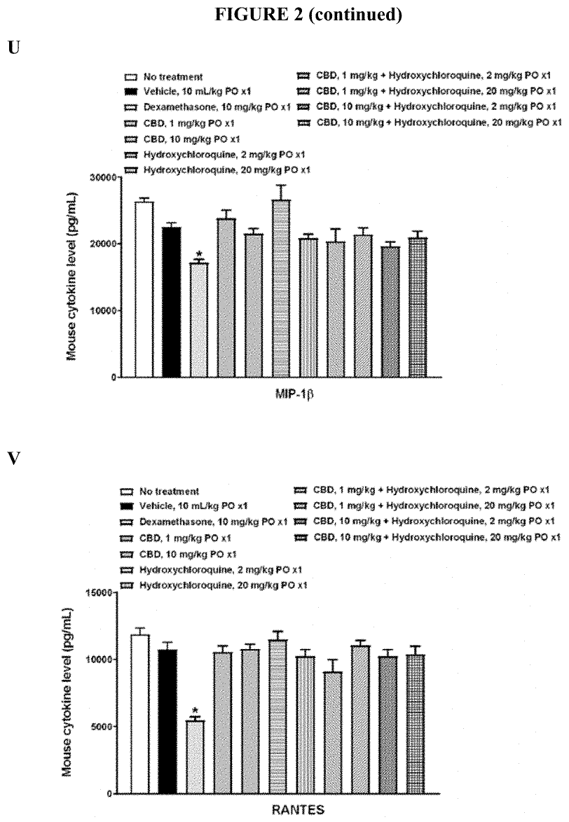

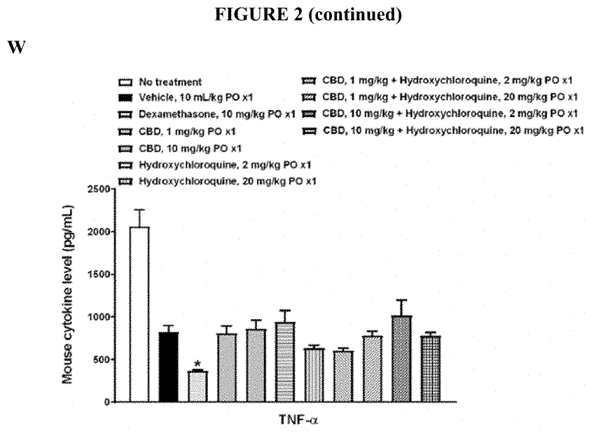

CBD given at 10 mg/kg or hydroxychloroquine given at 20 mg/kg by oral gavage (PO) alone showed moderate suppression on the LPS-induced IL-1α, IL-1β, IL-2, IL-4, IL-5, and IL-9 release compared to the vehicle group. CBD at 1 mg/kg or hydroxychloroquine at 2 mg/kg PO alone had little or no effect on biomarker levels (FIG. 2; Tables 13 and 14).

CBD given at 1 mg/kg in combination with hydroxychloroquine given at 2 mg/kg by PO showed significant (p<0.05) reduction in IL-1α, IL-2, IL-4 and GM-CSF levels and moderate effect in IL-1β, IL-5, IL-9, IL-13 and IFN-γ compared to the vehicle group (FIG. 2; Tables 15 and 17).

CBD given at 10 mg/kg in combination with hydroxychloroquine given at 2 mg/kg by PO showed significant (p<0.05) effect on IL-1α level and moderate effect on IL-4, IL-5, IL-9 and IL-13 levels compared to the vehicle group (Tables 16 and 18). Moreover, when compared to the vehicle group, CBD at 10 mg/kg in combination with hydroxychloroquine at 20 mg/kg PO produced significant (p<0.05) effect on IL-4 level and moderate effect on IL-1α, IL-1β, IL-5, IL-9, IL-13 and IFN-γ in LPS-induced sepsis model (FIG. 2; Tables 16 and 18).

As expected, the positive control, dexamethasone at 10 mg/kg PO significantly (p<0.05) reduced the levels of levels of IL-1β, IL-2, IL-4, IL-5, IL-6, IL-10, IL-12(p40), IL-12(p70), IL-17A, Eotaxin, GM-CSF, MCP-1, MIP-1α, MIP-1β, RANTES, and TNF-α compared to the vehicle group in the study (FIG. 2; Tables 11 and 12).

### Example 6—Combination of CBD and Hydroxychloroquine Synergise to Modulate an Inflammatory Response In Vitro

CBD and hydroxychloroquine act synergistically to inhibit LPS-induced production of the inflammatory cytokines IL-1p, IL-6, TNF-α, IL-1α, and MIP-1α at multiple drug concentrations. PBMCs from Donor 1 were treated with 5 μg/mL CBD in combination with 0.63, 1.25, 2.5, 5 and 10 μg/mL hydroxychloroquine, the results of which are shown in Table 19. PBMCs from Donor 2 were treated with 5 μg/mL CBD in combination with 0.63, 1.25, 2.5, 5, 10 and/or 20 μg/mL hydroxychloroquine, the results of which are shown in Table 20.

Values presented herein are expressed as inhibition relative to the untreated control, i.e. a value of 0 is no inhibition and a value of 1 is complete inhibition of release of the relevant cytokine. Cell viability was greater than 84% in all of the treatments listed in Tables 19 and 20.

### Example 7—Combination of CBD and Hydroxychloroquine Synergise to Modulate an Inflammatory Response In Vivo

CBD and hydroxychloroquine act synergistically to inhibit the LPS-induced production of inflammatory cytokines IL-1β, IL-6, IL-12(p70), IFN-γ, and/or TNF-α at multiple drug concentrations (Tables 21-24).

### Example 8—Combination of CBD and Hydroxychloroquine Synergise to Modulate the Immune Response in an In Vivo Model of Pulmonary Inflammation

Mice were challenged with LPS intratracheally to trigger pulmonary inflammation in a manner recapitulates clinical aspects of pulmonary inflammation observed inflammatory respiratory conditions such as COPD (see, e.g., Hakannsson et al., 2012, Pulmonary Pharmacology & Therapeutics, 25: 399-406).

CBD and hydroxychloroquine act synergistically to inhibit the production of inflammatory cytokines IL-1β, IL-6, TNF-α, CXCL-1 and MCP-1 at multiple concentrations, as measured from the BALF of mice with LPS-induced pulmonary inflammation (Tables 25-26).

BALF collected from mice were also analysed for white blood cell (WBC) count using an automatic haematology analyser. Cell counts were normalised using sham treated mice, and analysed relative to the highest values across the groups (Table 27). These data demonstrate that the combination of CBD and hydroxychloroquine reduced total WBC counts and neutrophil levels to a greater extent than either CBD or hydroxychloroquine alone.

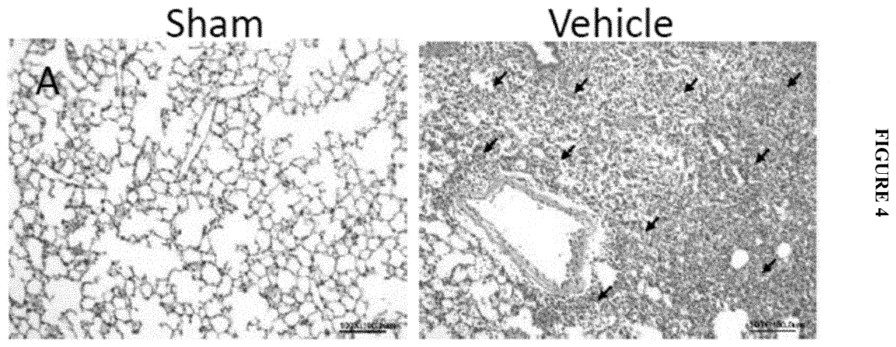

The combination of CBD and hydroxychloroquine also outperformed either CBD or hydroxychloroquine alone at equivalent doses in reducing inflammatory lesions in the lungs of mice with LPS-induced pulmonary inflammation (Tables 30 and 31; FIG. 4).

### Example 9—Combination of CBD and Hydroxychloroquine Synergise to Treat Inflammatory Bowel Disease In Vivo

CBD and hydroxychloroquine act synergistically to reduce the myeloperoxidase (MPO) levels in the colon tissue, stool consistency score and macroscopic damage score (Table 27).

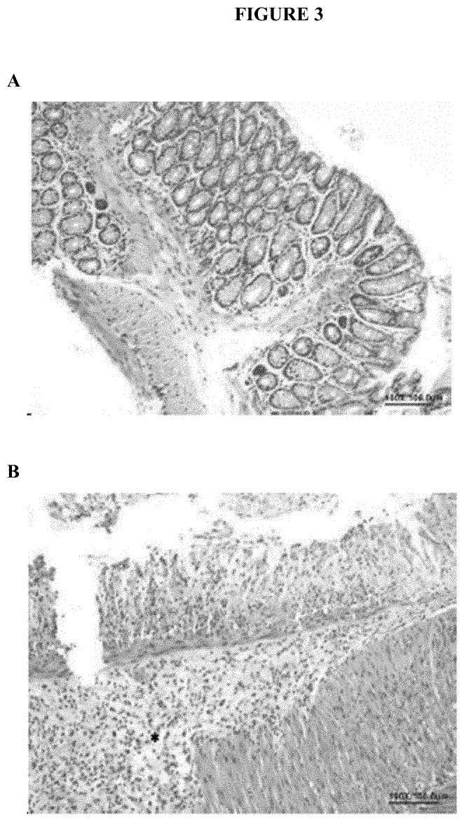

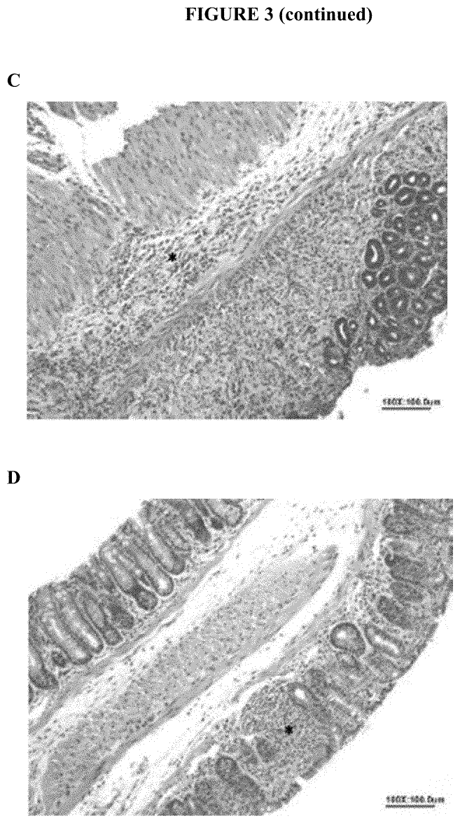

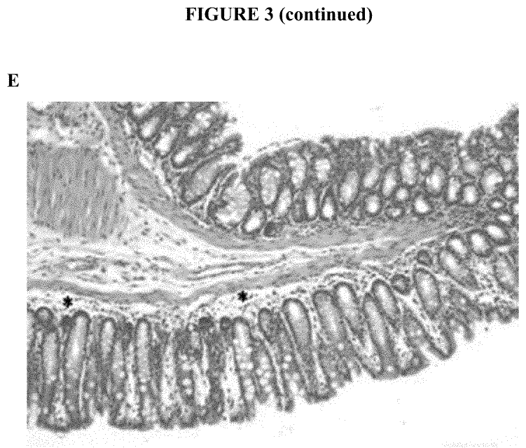

The combination of CBD and hydroxychloroquine was shown to outperform each drug alone in relation to a reduction in Total Colitis Index, as assessed by histological analysis of changes to the colitis of the distant colon of mice with TNBS-induced colitis (Tables 28). Representative stained sections are presented in FIG. 3. The combination of CBD and hydroxychloroquine also improved colon weight to body weight ratio (Table 28), as compared to the administration of either drug as a single agent.

### Example 10—Combination of CBD and Hydroxychloroquine Synergise to Treat Arthritis In Vivo

CBD and hydroxychloroquine at doses of 1 mg/kg and 2.5 mg/kg, respectively, act synergistically to reduce clinical score and paw volume at Day 24, together with pannus formation and total histological score at endpoint (i.e., Day 30). The Excess Over Bliss scores for clinical score, paw volume, pannus formation and total histological score were 0.05, 0.26, 0.30 and 0.03, respectively (Table 35).

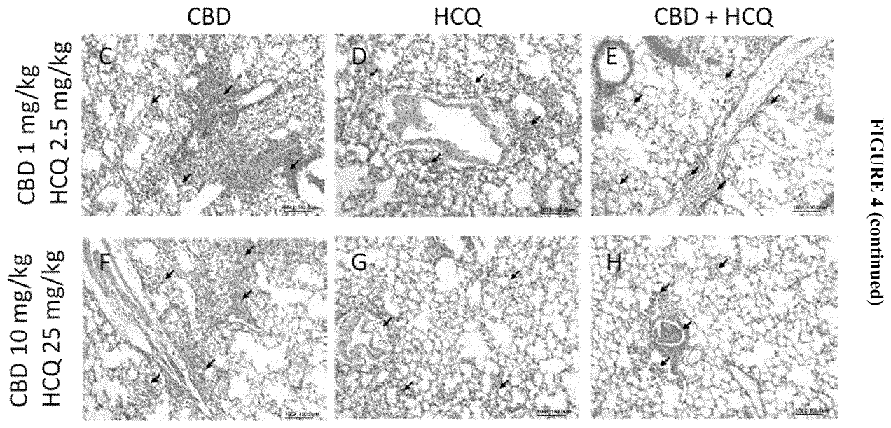

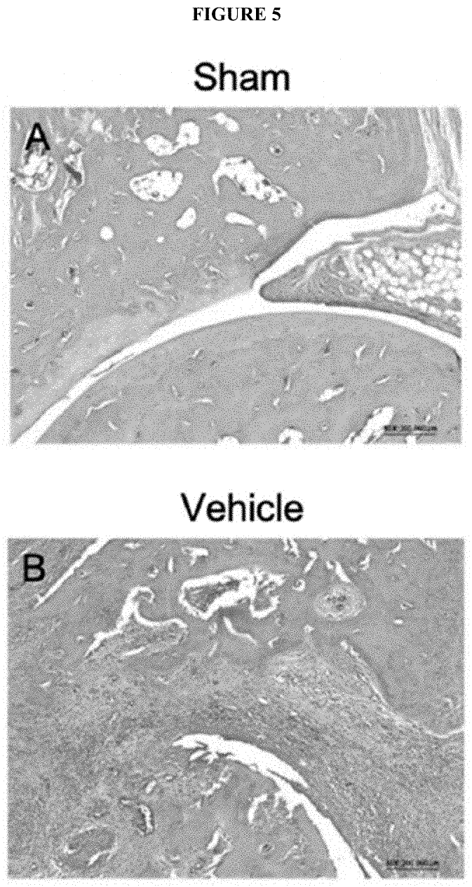

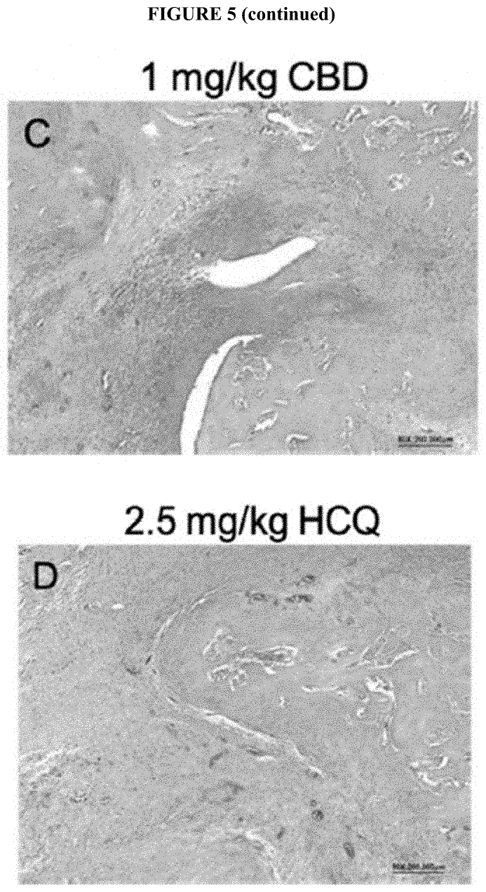

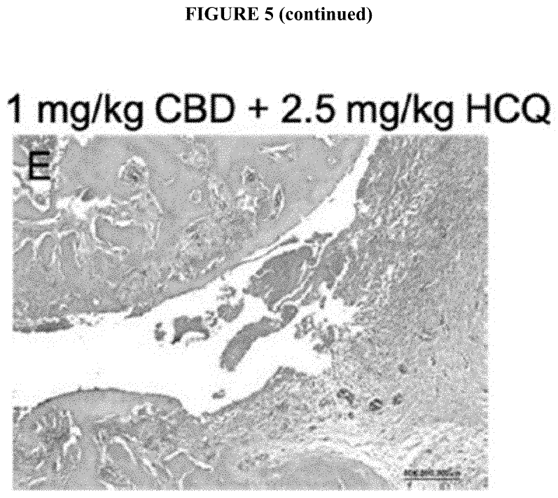

The combination of 1 mg/kg CBD and 2.5 mg/kg hydroxychloroquine was shown to outperform each drug alone in reducing levels of the inflammatory cytokines IL-10 and IL-6 in serum (Table 36; FIG. 5).

Hydroxychloroquine has been used for the treatment of rheumatoid arthritis in the form of hydroxychloroquine sulfate. However, long-term use of hydroxychloroquine has been associated with ocular toxicity and cardiac effects (e.g., cardiomyopathy and QT prolongation). Clinically, the most important predictor of ocular toxicity and cardiac effects in rheumatoid arthritis patients is the cumulative dose of hydroxychloroquine. To understand the capacity of CBD to permit the reduction of hydroxychloroquine, while retaining therapeutic effect, the results obtained using 1 mg/kg CBD in combination with 2.5 mg/kg hydroxychloroquine (i.e., low dose HCQ) was compared to that 25 mg/kg hydroxychloroquine alone (i.e., high dose HCQ). The combination of 1 mg/kg CBD and low dose HCQ was more, or similarly effective in reducing arthritis across all assessments, with the exception of mononuclear cell infiltration (Table 37). These data indicate that the combination of CBD with hydroxychloroquine allows for a ten-fold reduction in the dose of hydroxychloroquine without sacrificing therapeutic efficacy.

### SUMMARY

Collectively, these data demonstrate that the combination of CBD and hydroxychloroquine synergise to significantly modulate the inflammatory response in vitro and in vivo. The methods described herein have been reduced to practice in methods for modulating an inflammatory response, methods for the treatment of an inflammatory condition, such as sepsis-induced ARDS, pulmonary inflammation that is characteristic of inflammatory respiratory conditions such as COPD, asthma and CF, inflammatory bowel disease and arthritis. In this context, the combination of CBD and hydroxychloroquine can be used to modulate inflammatory mediators of sepsis-induced ARDS and pulmonary inflammation, including a reduction in IL-1β, which is known to be elevated in both the bronchoalveolar lavage fluid and in the circulating plasma ARDS patients (see, e.g., Meduri et al., 2009, Chest, 136: 1631-1643). Further, the combination of CBD and hydroxychloroquine has been shown to synergise to the extent that makes it possible to reduce the dose of hydroxychloroquine to a level that may minimise adverse effects associated with long-term use, while still eliciting a therapeutic effect.

TABLE 4
Study design for intraperitoneal administration of active agents

| | | | | Dosage | | |
|---|---|---|---|---|---|---|
| | | | Conc. | mL/ | mg/ | Mice |
| Group | Test Article | Route | mg/mL | kg | kg | (male) |
| 1 | Sham | NA | NA | NA | NA | 10 |
| 2 | No treatment | NA | NA | NA | NA | 10 |
| 3 | Vehicle for CBD | IP | NA | 10 | NA, x 1 | 10 |
| 4 | CBD | IP | 0.1 | 10 | 1, x 1 | 10 |
| 5 | CBD | IP | 0.5 | 10 | 5, x 1 | 10 |
| 6 | CBD | IP | 1 | 10 | 10, x 1 | 10 |
| 7 | CBD | IP | 2.5 | 10 | 25, x 1 | 10 |
| 8 | CBD | IP | 5 | 10 | 50, x 1 | 10 |
| 9 | CBD | IP | 10 | 10 | 100, x 1 | 10 |
| 10 | Vehicle for | IP | NA | 10 | NA, x 1 | 10 |
| | Hydroxychloroquine | | | | | |
| 11 | Hydroxychloroquine | IP | 0.1 | 10 | 1, x 1 | 10 |
| 12 | Hydroxychloroquine | IP | 0.5 | 10 | 5, x 1 | 10 |
| 13 | Hydroxychloroquine | IP | 1 | 10 | 10, x 1 | 10 |
| 14 | Hydroxychloroquine | IP | 2.5 | 10 | 25, x 1 | 10 |
| 15 | Hydroxychloroquine | IP | 5 | 10 | 50, x 1 | 10 |
| 16 | Hydroxychloroquine | IP | 10 | 10 | 100, x 1 | 10 |
| 17 | Dexamethasone | PO | 1 | 10 | 10, x 1 | 10 |

TABLE 5
Study design for oral administration of active agents

| | | | | Dosage | | |
|---|---|---|---|---|---|---|
| | | | Conc. | mL/ | mg/ | Mice |
| Group | Test Article | Route | mg/mL | kg | kg | (male) |
| 1 | No treatment^{b} | NA | NA | NA | NA | 10 |
| 2 | Vehicle^{c} | PO | NA | 10 | NA, x 1 | 10 |
| 3 | Dexamethasone^{d} | PO | 1 | 10 | 10, x 1 | 10 |
| 4 | CBD^{c} | PO | 0.1 | 10 | 1, x 1 | 10 |
| 5 | CBD^{c} | PO | 1 | 10 | 10, x 1 | 10 |
| 6 | Hydroxychloroquine^{c} | PO | 0.2 | 10 | 2, x 1 | 10 |
| 7 | Hydroxychloroquine^{c} | PO | 2 | 10 | 20, x 1 | 10 |
| 8 | CBD low + | PO + | 0.1 + | 10 + | 1, x 1 + | 10 |
| | Hydroxychloroquine | PO | 0.2 | 10 | 2, x 1 | |
| | low^{c} | | | | | |
| 9 | CBD low + | PO + | 0.1 + | 10 + | 1, x 1 + | 10 |
| | Hydroxychloroquine | PO | 2 | 10 | 20, x 1 | |
| | high^{c} | | | | | |
| 10 | CBD high + | PO + | 1 + | 10 + | 10, x 1 + | 10 |
| | Hydroxychloroquine | PO | 0.2 | 10 | 2, x 1 | |
| | low^{c} | | | | | |
| 11 | CBD high + | PO + | 1 + | 10 + | 10, x 1 + | 10 |
| | Hydroxychloroquine | PO | 2 | 10 | 20, x 1 | |
| | high^{c} | | | | | |

^{a}Male C57BL/6J mice weighing 18 to 20 g are used in the study.
^{b}LPS challenge, no other treatment
^{c}Vehicle for test articles (TAs): (sesame oil for CBD and PBS for Hydrosychloroquine). Both test articles CBD and Hydroxychloroquine are formulated in sesame oil or PBS for dosing.
Vehicle and TAs, alone or in combination, are administered orally (PO) at 1 hr before LPS (100 μg/mouse) injected intravenously (IV).
^{d}Dexamethasone: vehicle = 1% Tween 80/Water, is dosed PO at 1 hr prior to LPS.
^{e}At 2 hrs after LPS injection, blood is collected from all mice by cardiac puncture, sera processed and assayed for biomarkers using 23-plex for IL-1α, IL-1β, IL-2, IL-3, IL-4, IL-5, IL0-6, IL-9, IL-10, IL-12p40, IL-12p70, IL-13, IL-17A, Eotaxin, G-CSF, GM-CSF, IFN-γ, KC, MCP-1(MCAF), MIP-1α, MIP-1β, RANTES, and TNF-α.

TABLE 6
Results from immune marker panel of mice with LPS-induced sepsis
following IP administration of CBD or hydroxychloroquine

| | | | | | | [symbol] | | | | | |
|---|---|---|---|---|---|---|---|---|---|---|---|
| Gr. | Treatment | Route | Dose | No. | [symbol] | [symbol] | [symbol] | [symbol] | [symbol] | [symbol] | [symbol] |
| 1 | [symbol] | NA | NA | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 2 | no | NA | NA | 1 | [symbol] | [symbol] | [symbol] | [symbol] | [symbol] | [symbol] | [symbol] |
| | treatment | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 3 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 4 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |

Mean ± SEM values of biomarkers were determined, and one-way ANOVA followed by Dunnett's test was applied for comparison between the vehicle and treated groups. Differences are considered significant at [symbol] p < 0.05, vs [symbol] *p < 0.05, vs vehicle for CBD (Group 2); [symbol] p < 0.05, vs vehicle for Hydroxychloroquine (Group 10). [symbol]
indicates data missing or illegible when filed

TABLE 7
Results from immune marker panel of mice with
LPS-induced sepsis following IP administration
of CBD or hydroxychloroquine (continued)

| | | | | | | [symbol] | | | | | |
|---|---|---|---|---|---|---|---|---|---|---|---|
| Gr. | Treatment | Route | Dose | No. | [symbol] | [symbol] | [symbol] | [symbol] | [symbol] | [symbol] | [symbol] |
| 5 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 6 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 7 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 8 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |

Mean ± SEM values of biomarkers were determined, and one-way ANOVA followed by Dunnett's test was applied for comparison between the vehicle and treated groups. Differences are considered significant at [symbol] p < 0.05, vs [symbol] *p < 0.05, vs vehicle for CBD (Group 2); [symbol] p < 0.05, vs vehicle for Hydroxychloroquine (Group 10). [symbol]
indicates data missing or illegible when filed

TABLE 8
Results from immune marker panel of mice with
LPS-induced sepsis following IP administration
of CBD or hydroxychloroquine (continued)

| | | | | | | [symbol] | | | | | |
|---|---|---|---|---|---|---|---|---|---|---|---|
| Gr. | Treatment | Route | Dose | No. | [symbol] | [symbol] | [symbol] | [symbol] | [symbol] | [symbol] | [symbol] |
| 9 | [symbol] | [symbol] | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 10 | Vehicle for | [symbol] | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 11 | [symbol] | | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 12 | [symbol] | [symbol] | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |

Mean ± SEM values of biomarkers were determined, and one-way ANOVA followed by Dunnett's test was applied for comparison between the vehicle and treated groups. Differences are considered significant at [symbol] p < 0.05, vs [symbol] *p < 0.05, vs vehicle for CBD (Group 2); [symbol] p < 0.05, vs vehicle for Hydroxychloroquine (Group 10). [symbol]
indicates data missing or illegible when filed

TABLE 9
Results from immune marker panel of mice with
LPS-induced sepsis following IP administration
of CBD or hydroxychloroquine (continued)

| | | | | | | [symbol] | | | | | |
|---|---|---|---|---|---|---|---|---|---|---|---|
| Gr. | Treatment | Route | Dose | No. | [symbol] | [symbol] | [symbol] | [symbol] | [symbol] | [symbol] | [symbol] |
| 13 | [symbol] | [symbol] | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 14 | [symbol] | [symbol] | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 15 | [symbol] | [symbol] | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |
| 16 | [symbol] | [symbol] | [symbol] | 1 | [symbol] | [symbol] | [symbol] | [symbol] | [symbol] | [symbol] | [symbol] |
| | Hydroxy- | | | 2 | [symbol] | [symbol] | [symbol] | [symbol] | [symbol] | [symbol] | [symbol] |
| | chloro- | | | 3 | [symbol] | [symbol] | [symbol] | [symbol] | [symbol] | [symbol] | [symbol] |
| | quine | | | 4 | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |

Mean ± SEM values of biomarkers were determined, and one-way ANOVA followed by Dunnett's test was applied for comparison between the vehicle and treated groups. Differences are considered significant at [symbol] p < 0.05, vs [symbol] *p < 0.05, vs vehicle for CBD (Group 2); [symbol] p < 0.05, vs vehicle for Hydroxychloroquine (Group 10). [symbol]
indicates data missing or illegible when filed

TABLE 10
Results from immune marker panel of mice with LPS-induced sepsis
following IP administration of dexamethasone (positive control)

| | | | | | | [symbol] | | | | | |
|---|---|---|---|---|---|---|---|---|---|---|---|
| Gr. | Treatment | Route | Dose | No. | [symbol] | [symbol] | [symbol] | [symbol] | [symbol] | [symbol] | [symbol] |
| 17 | [symbol] | PO | [symbol] | 1 | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] |

Mean ± SEM values of biomarkers were determined, and one-way ANOVA followed by Dunnett's test was applied for comparison between the vehicle and treated groups. Differences are considered significant at [symbol] p < 0.05, vs [symbol] *p < 0.05, vs vehicle for CBD (Group 2); [symbol] p < 0.05, vs vehicle for Hydroxychloroquine (Group 10). [symbol]
indicates data missing or illegible when filed

TABLE 11
Results from immune marker panel of mice with LPS-induced sepsis
following oral administration of positive and negative controls

| | | | | | [symbol] | | | | | | | | | | | | |
|---|---|---|---|---|---|---|---|---|---|---|---|---|---|---|---|---|---|
| Gr. | Treatment | Route | Dose | No. | [symbol] | [symbol] | [symbol] | [symbol] | [symbol] | [symbol] | [symbol] | [symbol] | [symbol] | [symbol] | [symbol] | [symbol] | [symbol] |
| 1 | No | NA | NA | 1 | [symbol] | [symbol] | [symbol] | [symbol] | [symbol] | [symbol] | [symbol] | [symbol] | [symbol] | [symbol] | [symbol] | [symbol] | [symbol] |
| | treatment | | | 2 | [symbol] | [symbol] | [symbol] | [symbol] | [symbol] | [symbol] | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 2 | [symbol] | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 3 | [symbol] | PO | [symbol] | 1 | [symbol] | [symbol] | [symbol] | [symbol] | [symbol] | [symbol] | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 12
Results from immune marker panel of mice with LPS-induced sepsis following
oral administration of positive and negative controls (continued)

| | | | | | [symbol] | | | | | | | | | |
|---|---|---|---|---|---|---|---|---|---|---|---|---|---|---|
| Gr. | Treatment | Route | Dose | No. | Eotaxin | G-CSF | GM-CSF | IFN [symbol] | KC | M [symbol] | N [symbol] | [symbol] | RANTES | TNF [symbol] |
| 1 | No | NA | NA | 1 | [symbol] | [symbol] | [symbol] | [symbol] | [symbol] | [symbol] | [symbol] | [symbol] | [symbol] | [symbol] |
| | treatment | | | 2 | [symbol] | [symbol] | [symbol] | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] |
| 2 | Vehicle | PO + | [symbol] + | 1 | [symbol] | [symbol] | [symbol] | [symbol] | [symbol] | [symbol] | [symbol] | [symbol] | [symbol] | [symbol] |
| | [symbol] + | PO | [symbol] | 2 | [symbol] | [symbol] | [symbol] | [symbol] | [symbol] | [symbol] | [symbol] | [symbol] | [symbol] | [symbol] |
| | [symbol] | | | 3 | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] |
| 3 | [symbol] | PO | [symbol] | 1 | [symbol] | [symbol] | [symbol] | [symbol] | [symbol] | [symbol] | [symbol] | [symbol] | [symbol] | [symbol] |
| | | | | 2 | [symbol] | [symbol] | [symbol] | [symbol] | [symbol] | [symbol] | [symbol] | [symbol] | [symbol] | [symbol] |
| | | | | 3 | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 13
Results from immune marker panel of mice with LPS-induced sepsis
following oral administration of CBD or hydroxychloroquine

| | | | | | [symbol] | | | | | | | | | | | | |
|---|---|---|---|---|---|---|---|---|---|---|---|---|---|---|---|---|---|
| Gr. | Treatment | Route | Dose | No. | [symbol] | [symbol] | [symbol] | [symbol] | [symbol] | [symbol] | [symbol] | [symbol] | [symbol] | [symbol] | [symbol] | [symbol] | [symbol] |
| 4 | [symbol] + | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | Vehicle | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | [symbol] | | | 3 | [symbol] | [symbol] | [symbol] | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 5 | [symbol] + | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | Vehicle | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | [symbol] | | | 3 | [symbol] | [symbol] | [symbol] | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 6 | [symbol] | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | Hydroxychloroquine | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | [symbol] + | | | 3 | [symbol] | [symbol] | [symbol] | [symbol] | [symbol] | [symbol] | [symbol] | [symbol] | [symbol] | [symbol] | [symbol] | [symbol] | [symbol] |
| | Vehicle | | | 4 | [symbol] | [symbol] | [symbol] | [symbol] | [symbol] | [symbol] | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 14
Results from immune marker panel of mice with LPS-induced sepsis following
oral administration of CBD or hydroxychloroquine (continued)

| | | | | | [symbol] | | | | | | | | | |
|---|---|---|---|---|---|---|---|---|---|---|---|---|---|---|
| Gr. | Treatment | Route | Dose | No. | Eotaxin | G-CSF | GM-CSF | IFN [symbol] | KC | M [symbol] | N [symbol] | [symbol] | RANTES | TNF [symbol] |
| 4 | [symbol] + | PO + | [symbol] + | 1 | [symbol] | [symbol] | [symbol] | [symbol] | [symbol] | [symbol] | [symbol] | [symbol] | [symbol] | [symbol] |
| | Vehicle | PO | [symbol] | 2 | [symbol] | [symbol] | [symbol] | [symbol] | [symbol] | [symbol] | [symbol] | [symbol] | [symbol] | [symbol] |
| | [symbol] | | | 3 | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] |
| 5 | [symbol] + | PO + | [symbol] + | 1 | [symbol] | [symbol] | [symbol] | [symbol] | [symbol] | [symbol] | [symbol] | [symbol] | [symbol] | [symbol] |
| | Vehicle | PO | [symbol] | 2 | [symbol] | [symbol] | [symbol] | [symbol] | [symbol] | [symbol] | [symbol] | [symbol] | [symbol] | [symbol] |
| | [symbol] | | | 3 | [symbol] | [symbol] | [symbol] | [symbol] | [symbol] | [symbol] | [symbol] | [symbol] | [symbol] | [symbol] |
| | | | | 4 | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] |
| 6 | [symbol] | PO + | [symbol] + | 1 | [symbol] | [symbol] | [symbol] | [symbol] | [symbol] | [symbol] | [symbol] | [symbol] | [symbol] | [symbol] |
| | Hydroxychloroquine | PO | [symbol] | 2 | [symbol] | [symbol] | [symbol] | [symbol] | [symbol] | [symbol] | [symbol] | [symbol] | [symbol] | [symbol] |
| | [symbol] + | | | 3 | [symbol] | [symbol] | [symbol] | [symbol] | [symbol] | [symbol] | [symbol] | [symbol] | [symbol] | [symbol] |
| | Vehicle | | | 4 | [symbol] | [symbol] | [symbol] | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 15
Results from immune marker panel of mice with LPS-induced sepsis following
oral administration of combination of CBD and hydroxychloroquine

| | | | | | [symbol] | | | | | | | | | | | | |
|---|---|---|---|---|---|---|---|---|---|---|---|---|---|---|---|---|---|
| Gr. | Treatment | Route | Dose | No. | [symbol] | [symbol] | [symbol] | [symbol] | [symbol] | [symbol] | [symbol] | [symbol] | [symbol] | [symbol] | [symbol] | [symbol] | [symbol] |
| 7 | [symbol] | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | Hydroxychloroquine | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | [symbol] + | | | 3 | [symbol] | [symbol] | [symbol] | [symbol] | [symbol] | [symbol] | [symbol] | [symbol] | [symbol] | [symbol] | [symbol] | [symbol] | [symbol] |
| | Vehicle | | | 4 | [symbol] | [symbol] | [symbol] | [symbol] | [symbol] | [symbol] | [symbol] | [symbol] | [symbol] | [symbol] | [symbol] | [symbol] | [symbol] |
| | [symbol] | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 8 | [symbol] + | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | [symbol] | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | Hydroxychloroquine | | | 3 | [symbol] | [symbol] | [symbol] | [symbol] | [symbol] | [symbol] | [symbol] | [symbol] | [symbol] | [symbol] | [symbol] | [symbol] | [symbol] |
| | [symbol] | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 9 | [symbol] + | PO + | [symbol] + | 1 | [symbol] | [symbol] | [symbol] | [symbol] | [symbol] | [symbol] | [symbol] | [symbol] | [symbol] | [symbol] | [symbol] | [symbol] | [symbol] |
| | [symbol] | PO | [symbol] | 2 | [symbol] | [symbol] | [symbol] | [symbol] | [symbol] | [symbol] | [symbol] | [symbol] | [symbol] | [symbol] | [symbol] | [symbol] | [symbol] |
| | Hydroxychloroquine | | | 3 | [symbol] | [symbol] | [symbol] | [symbol] | [symbol] | [symbol] | [symbol] | [symbol] | [symbol] | [symbol] | [symbol] | [symbol] | [symbol] |
| | [symbol] | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] | [symbol] | [symbol] | [symbol] | [symbol] | [symbol] | [symbol] | [symbol] | [symbol] | [symbol] | [symbol] |
| | | | | 6 | [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 16
Results from immune marker panel of mice with LPS-induced sepsis following
oral administration of combination of CBD and hydroxychloroquine (cont.)

| | | | | | [symbol] | | | | | | | | | | | | |
|---|---|---|---|---|---|---|---|---|---|---|---|---|---|---|---|---|---|
| Gr. | Treatment | Route | Dose | No. | [symbol] | [symbol] | [symbol] | [symbol] | [symbol] | [symbol] | [symbol] | [symbol] | [symbol] | [symbol] | [symbol] | [symbol] | [symbol] |
| 10 | [symbol] + | PO + | [symbol] + | 1 | 87. [symbol] | [symbol] | [symbol] | 37.2 | 18.3 | [symbol] | [symbol] | [symbol] | [symbol] | [symbol] | [symbol] | [symbol] | [symbol] |
| | [symbol] | PO | [symbol] | 2 | [symbol] | [symbol] | [symbol] .3 | [symbol] .9 | [symbol] | [symbol] | [symbol] | [symbol] | [symbol] | [symbol] | [symbol] | [symbol] | [symbol] |
| | Hydroxychloroquine | | | 3 | [symbol] .2 | [symbol] | [symbol] | 2 [symbol] | [symbol] | [symbol] | [symbol] | [symbol] | [symbol] | [symbol] | [symbol] | [symbol] | [symbol] |
| | [symbol] | | | 4 | [symbol] | [symbol] | 27. [symbol] | [symbol] | [symbol] | [symbol] | [symbol] | [symbol] | [symbol] | [symbol] | [symbol] | [symbol] | [symbol] |
| | | | | 5 | [symbol] | [symbol] | [symbol] .2 | 36 [symbol] | 18 [symbol] | [symbol] | [symbol] | [symbol] | [symbol] | [symbol] | [symbol] | [symbol] | [symbol] |
| | | | | 6 | [symbol] | [symbol] | 2 [symbol] | 37 [symbol] | [symbol] | [symbol] | [symbol] | [symbol] | [symbol] | [symbol] | [symbol] | [symbol] | [symbol] |
| | | | | 7 | [symbol] | [symbol] | [symbol] .2 | 27 [symbol] | [symbol] | [symbol] .7 | [symbol] | [symbol] | [symbol] | [symbol] | [symbol] | [symbol] | [symbol] |
| | | | | 8 | [symbol] .4 | [symbol] | [symbol] | [symbol] | [symbol] | [symbol] | [symbol] | [symbol] | [symbol] | [symbol] | [symbol] | [symbol] | [symbol] |
| | | | | 9 | [symbol] | [symbol] | [symbol] .2 | [symbol] | 28 [symbol] | [symbol] .8 | [symbol] | [symbol] | [symbol] | [symbol] | [symbol] | [symbol] | [symbol] |
| | | | | 10 | [symbol] | [symbol] | 27 [symbol] | [symbol] | [symbol] | [symbol] | [symbol] | [symbol] | [symbol] | [symbol] | [symbol] | [symbol] | [symbol] |
| | | | | Mean | [symbol] | [symbol] | [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |
| 11 | [symbol] + | PO + | [symbol] + | 1 | [symbol] .5 | [symbol] .2 | 2 [symbol] .1 | [symbol] | [symbol] | [symbol] | [symbol] | [symbol] | [symbol] | [symbol] | [symbol] | [symbol] | [symbol] .8 |
| | [symbol] | PO | [symbol] | 2 | [symbol] | [symbol] | [symbol] | [symbol] | [symbol] .2 | [symbol] | [symbol] | [symbol] | [symbol] | [symbol] | [symbol] | [symbol] | [symbol] |
| | Hydroxychloroquine | | | 3 | [symbol] | [symbol] .2 | [symbol] .3 | [symbol] | [symbol] | [symbol] | [symbol] | [symbol] | [symbol] | [symbol] | [symbol] | [symbol] | [symbol] |
| | [symbol] | | | 4 | [symbol] | [symbol] | [symbol] | [symbol] | [symbol] | [symbol] | [symbol] | [symbol] | [symbol] | [symbol] | [symbol] | [symbol] | [symbol] |
| | | | | 5 | [symbol] | [symbol] .8 | 2 [symbol] | [symbol] | [symbol] | 4 [symbol] | [symbol] | [symbol] | [symbol] | [symbol] | [symbol] | [symbol] | [symbol] |
| | | | | 6 | 6 [symbol] | [symbol] | [symbol] | [symbol] | [symbol] | [symbol] | [symbol] | [symbol] | [symbol] | [symbol] | [symbol] | [symbol] | [symbol] |
| | | | | 7 | [symbol] | [symbol] | 2 [symbol] | [symbol] | [symbol] | [symbol] | [symbol] | [symbol] | [symbol] | [symbol] | [symbol] | [symbol] | [symbol] |
| | | | | 8 | [symbol] | [symbol] | [symbol] | [symbol] | [symbol] .7 | [symbol] | [symbol] | [symbol] | [symbol] | [symbol] | [symbol] | [symbol] | [symbol] |
| | | | | 9 | [symbol] .6 | [symbol] .2 | 2 [symbol] | [symbol] | [symbol] | [symbol] | [symbol] | [symbol] | [symbol] | [symbol] | [symbol] | [symbol] | [symbol] |
| | | | | 10 | [symbol] | [symbol] | [symbol] .3 | [symbol] | [symbol] | [symbol] | [symbol] | [symbol] | [symbol] | [symbol] | [symbol] | [symbol] | [symbol] |
| | | | | Mean | [symbol] | [symbol] .2 | 27 [symbol] | [symbol] | [symbol] | [symbol] | [symbol] | [symbol] | [symbol] | [symbol] | [symbol] | [symbol] | [symbol] |
| | | | | SEM | [symbol] | [symbol] | [symbol] | [symbol] | [symbol] | [symbol] | [symbol] | [symbol] | [symbol] | [symbol] | [symbol] | [symbol] | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 17
Results from immune marker panel of mice with LPS-induced sepsis following
oral administration of combination of CBD and hydroxychloroquine (cont.)

| | | | | | [symbol] | | | | | | | | | |
|---|---|---|---|---|---|---|---|---|---|---|---|---|---|---|
| Gr. | Treatment | Route | Dose | No. | Eotaxin | G-CSF | GM-CSF | IFN [symbol] | KC | M [symbol] | N [symbol] | [symbol] | RANTES | TNF [symbol] |
| 7 | [symbol] | PO + | [symbol] + | 1 | [symbol] .5 | [symbol] | 3 [symbol] | [symbol] | [symbol] .2 | [symbol] .2 | [symbol] | [symbol] | [symbol] | 8 [symbol] |
| | Hydroxychloroquine | PO | [symbol] | 2 | [symbol] | [symbol] | 2 [symbol] .9 | [symbol] | 2 [symbol] | [symbol] | [symbol] | [symbol] | [symbol] .7 | [symbol] .2 |
| | [symbol] + | | | 3 | [symbol] | [symbol] .8 | [symbol] | [symbol] | [symbol] | [symbol] | [symbol] | [symbol] | [symbol] | [symbol] .7 |
| | Vehicle | | | 4 | [symbol] .8 | 7 [symbol] | [symbol] | [symbol] | [symbol] .8 | [symbol] | [symbol] | [symbol] .1 | [symbol] .8 | [symbol] .4 |
| | [symbol] | | | 5 | [symbol] .1 | [symbol] .8 | 2 [symbol] | 18 [symbol] | 2 [symbol] | [symbol] | [symbol] .2 | [symbol] .2 | [symbol] .8 | [symbol] |
| | | | | 6 | 8 [symbol] | [symbol] .2 | 2 [symbol] .7 | [symbol] .8 | [symbol] | [symbol] .2 | [symbol] | 3 [symbol] | [symbol] | [symbol] .9 |
| | | | | 7 | [symbol] | [symbol] .8 | 2 [symbol] .2 | [symbol] | [symbol] .2 | [symbol] | [symbol] .2 | 1 [symbol] | [symbol] | [symbol] |
| | | | | 8 | [symbol] | [symbol] | [symbol] | [symbol] | [symbol] .9 | [symbol] | [symbol] | [symbol] | [symbol] | [symbol] |
| | | | | 9 | [symbol] .1 | [symbol] .2 | 2 [symbol] .2 | [symbol] | [symbol] | [symbol] | [symbol] | [symbol] .2 | [symbol] | [symbol] .7 |
| | | | | 10 | [symbol] .2 | [symbol] 7.7 | [symbol] | [symbol] | [symbol] | [symbol] .1 | [symbol] .4 | 2 [symbol] .2 | [symbol] | [symbol] |
| | | | | Mean | [symbol] | [symbol] .4 | [symbol] | [symbol] | [symbol] | 7 [symbol] .8 | [symbol] | [symbol] | 10 [symbol] | [symbol] .7 |
| | | | | SEM | [symbol] | [symbol] | [symbol] | 3. [symbol] | [symbol] .1 | [symbol] .7 | 7 [symbol] | [symbol] .8 | [symbol] .8 | [symbol] |
| 8 | [symbol] + | PO + | [symbol] + | 1 | [symbol] | [symbol] .8 | [symbol] .8 | [symbol] | [symbol] | [symbol] | [symbol] | 2 [symbol] | [symbol] | [symbol] |
| | [symbol] | PO | [symbol] | 2 | [symbol] | [symbol] | [symbol] | [symbol] | [symbol] | [symbol] | [symbol] | [symbol] | [symbol] | [symbol] |
| | Hydroxychloroquine | | | 3 | [symbol] | [symbol] | 2 [symbol] | [symbol] | [symbol] | [symbol] | [symbol] | [symbol] | [symbol] | [symbol] |
| | [symbol] | | | 4 | [symbol] | [symbol] | [symbol] | [symbol] | [symbol] | [symbol] | [symbol] | [symbol] | [symbol] | [symbol] |
| | | | | 5 | [symbol] .8 | 7 [symbol] | [symbol] | [symbol] | [symbol] | [symbol] | [symbol] | [symbol] | [symbol] | [symbol] |
| | | | | 6 | [symbol] .2 | [symbol] .7 | 3 [symbol] | [symbol] | [symbol] .7 | [symbol] | [symbol] | [symbol] | [symbol] .8 | [symbol] |
| | | | | 7 | 67 [symbol] | 79 [symbol] | [symbol] .2 | [symbol] | [symbol] | [symbol] | [symbol] | 3 [symbol] | [symbol] | [symbol] |
| | | | | 8 | [symbol] | [symbol] | [symbol] | [symbol] | [symbol] .7 | [symbol] | [symbol] | 2 [symbol] | [symbol] .2 | [symbol] .8 |
| | | | | 9 | [symbol] | 8 [symbol] | [symbol] | [symbol] | [symbol] .3 | [symbol] | [symbol] .4 | 2 [symbol] .7 | [symbol] .2 | [symbol] |
| | | | | 10 | [symbol] | [symbol] | [symbol] | [symbol] | [symbol] | [symbol] | [symbol] | [symbol] | [symbol] | [symbol] |
| | | | | Mean | 6 [symbol] .7 | [symbol] | [symbol] | [symbol] | [symbol] | [symbol] | [symbol] | [symbol] | [symbol] | [symbol] |
| | | | | SEM | [symbol] | [symbol] | 7. [symbol] | [symbol] .2 | 7 [symbol] .3 | [symbol] | [symbol] | [symbol] .3 | [symbol] | [symbol] |
| 9 | [symbol] + | PO + | [symbol] + | 1 | [symbol] | [symbol] 2.3 | 2 [symbol] | [symbol] | 27 [symbol] .3 | [symbol] | [symbol] | [symbol] | [symbol] | [symbol] |
| | [symbol] | PO | [symbol] | 2 | [symbol] .2 | [symbol] .3 | [symbol] | [symbol] | [symbol] | [symbol] | [symbol] .6 | [symbol] | [symbol] | [symbol] |
| | Hydroxychloroquine | | | 3 | [symbol] | [symbol] | [symbol] | [symbol] | [symbol] | [symbol] | [symbol] | [symbol] | [symbol] | [symbol] |
| | [symbol] | | | 4 | [symbol] | [symbol] | 3 [symbol] | [symbol] | [symbol] | [symbol] | [symbol] | [symbol] | [symbol] | 8 [symbol] .3 |
| | | | | 5 | [symbol] | 8 [symbol] | [symbol] | [symbol] | [symbol] | [symbol] | [symbol] | [symbol] | [symbol] | 7 [symbol] |
| | | | | 6 | [symbol] | [symbol] | [symbol] | [symbol] | [symbol] | [symbol] | [symbol] | [symbol] | [symbol] | [symbol] .8 |
| | | | | 7 | [symbol] | [symbol] | [symbol] | [symbol] | [symbol] | [symbol] | [symbol] | [symbol] | [symbol] | [symbol] |
| | | | | 8 | [symbol] | [symbol] .7 | [symbol] | [symbol] .2 | [symbol] | [symbol] .8 | [symbol] | [symbol] | [symbol] .4 | [symbol] |
| | | | | 9 | [symbol] .2 | 8 [symbol] | [symbol] | [symbol] | [symbol] | [symbol] | [symbol] | [symbol] | [symbol] | [symbol] |
| | | | | 10 | [symbol] | [symbol] .3 | [symbol] | [symbol] | [symbol] | [symbol] .7 | [symbol] | [symbol] | [symbol] | [symbol] |
| | | | | Mean | 7 [symbol] | [symbol] | 3 [symbol] .8 | [symbol] | [symbol] | [symbol] | [symbol] .9 | [symbol] | [symbol] | [symbol] |
| | | | | SEM | [symbol] | [symbol] | 3.7 | 2.2 | [symbol] | 3 [symbol] .9 | [symbol] | [symbol] .8 | [symbol] .7 | [symbol] |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 18
Results from immune marker panel of mice with LPS-induced sepsis following
oral administration of combination of CBD and hydroxychloroquine (cont.)

| | | | | | [symbol] | | | | | | | | | |
|---|---|---|---|---|---|---|---|---|---|---|---|---|---|---|
| Gr. | Treatment | Route | Dose | No. | Eotaxin | G-CSF | GM-CSF | IFN [symbol] | KC | M [symbol] | N [symbol] | [symbol] | RANTES | TNF [symbol] |
| 10 | [symbol] + | PO + | [symbol] + | 1 | 7 [symbol] .7 | 7 [symbol] | [symbol] | [symbol] | [symbol] | [symbol] | [symbol] | [symbol] | [symbol] .3 | [symbol] |
| | [symbol] | PO | [symbol] | 2 | [symbol] .3 | [symbol] | 2 [symbol] | [symbol] | [symbol] .8 | [symbol] | [symbol] | [symbol] .5 | [symbol] | [symbol] .9 |
| | Hydroxychloroquine | | | 3 | 7 [symbol] | 4 [symbol] | [symbol] | [symbol] | [symbol] .2 | [symbol] | [symbol] | 2 [symbol] | [symbol] | [symbol] |
| | [symbol] | | | 4 | [symbol] .9 | 68 [symbol] .4 | 2 [symbol] | [symbol] | [symbol] | [symbol] | [symbol] .3 | [symbol] .3 | [symbol] .3 | [symbol] |
| | | | | 5 | [symbol] .9 | [symbol] .8 | 28 [symbol] | [symbol] | [symbol] | [symbol] | [symbol] .3 | [symbol] .7 | [symbol] .3 | [symbol] |
| | | | | 6 | [symbol] | [symbol] .2 | [symbol] | [symbol] | [symbol] .8 | [symbol] | [symbol] | [symbol] | [symbol] | [symbol] .2 |
| | | | | 7 | [symbol] .7 | 7 [symbol] | [symbol] | [symbol] | [symbol] .8 | [symbol] | 2 [symbol] | [symbol] | [symbol] .2 | [symbol] .2 |
| | | | | 8 | [symbol] | [symbol] .1 | [symbol] | [symbol] | [symbol] .7 | [symbol] .5 | 2 [symbol] | [symbol] | [symbol] .7 | [symbol] .3 |
| | | | | 9 | 7 [symbol] | [symbol] .8 | [symbol] .9 | [symbol] | [symbol] | [symbol] | [symbol] .3 | [symbol] | [symbol] .5 | 8 [symbol] |
| | | | | 10 | 9 [symbol] | [symbol] .7 | [symbol] .9 | [symbol] | [symbol] | [symbol] | [symbol] | [symbol] | [symbol] | [symbol] |
| | | | | Mean | 7 [symbol] .7 | [symbol] | 3 [symbol] | [symbol] .7 | [symbol] | [symbol] | [symbol] | [symbol] | [symbol] | [symbol] |
| | | | | SEM | [symbol] | [symbol] | [symbol] .8 | 2.7 | [symbol] | [symbol] | [symbol] | [symbol] | [symbol] | [symbol] |
| 11 | [symbol] + | PO + | [symbol] + | 1 | [symbol] .2 | [symbol] | 2 [symbol] | [symbol] .2 | [symbol] | [symbol] | [symbol] | [symbol] | [symbol] | [symbol] |
| | [symbol] | PO | [symbol] | 2 | [symbol] | [symbol] | 3 [symbol] | [symbol] .3 | [symbol] | [symbol] | [symbol] | [symbol] | [symbol] | [symbol] .5 |
| | Hydroxychloroquine | | | 3 | 7 [symbol] | [symbol] | 3 [symbol] | [symbol] .3 | [symbol] .8 | [symbol] | [symbol] | [symbol] | [symbol] | [symbol] |
| | [symbol] | | | 4 | [symbol] | [symbol] 7.8 | [symbol] | [symbol] | [symbol] | [symbol] | [symbol] .7 | [symbol] .8 | [symbol] .8 | [symbol] |
| | | | | 5 | [symbol] | [symbol] | 2 [symbol] | [symbol] | [symbol] | [symbol] | [symbol] | [symbol] | [symbol] | [symbol] |
| | | | | 6 | 8 [symbol] | [symbol] | 3 [symbol] | 2 [symbol] .3 | [symbol] | [symbol] | [symbol] | [symbol] | [symbol] | [symbol] .7 |
| | | | | 7 | [symbol] | [symbol] | [symbol] .3 | [symbol] .4 | [symbol] | [symbol] | [symbol] .7 | [symbol] | [symbol] | [symbol] |
| | | | | 8 | 7 [symbol] .9 | [symbol] | 2 [symbol] .9 | [symbol] | [symbol] | [symbol] | [symbol] | [symbol] | [symbol] | [symbol] |
| | | | | 9 | [symbol] .8 | [symbol] | 2 [symbol] | [symbol] | [symbol] | [symbol] | [symbol] .7 | [symbol] | [symbol] | [symbol] |
| | | | | 10 | 7 [symbol] .4 | [symbol] | [symbol] .2 | [symbol] .4 | [symbol] .8 | [symbol] | 2 [symbol] | [symbol] | [symbol] .3 | [symbol] |
| | | | | Mean | [symbol] | [symbol] | [symbol] | [symbol] .5 | [symbol] | [symbol] | 2 [symbol] 2 | 2 [symbol] | [symbol] | [symbol] |
| | | | | SEM | 37 [symbol] | [symbol] | 4.8 | 2.8 | 2 [symbol] | [symbol] | [symbol] | [symbol] .2 | [symbol] | 28.3 |

Mean ± SEM values of biomarker were determined, and one-way ANOVA followed by Dunnett's test was applied for comparison between the vehicle and treated groups. Differences are considered significant at *p < 0.05, vs vehicle control. [symbol]
indicates data missing or illegible when filed

TABLE 19
Synergistic inhibition of IL-1β, IL-6, TNF-α, IL-1α, and MIP-
1α release by CBD and HCQ in human PBMCs (Donor 1)

| | CBD | | HCQ | | | Ob- | |
|---|---|---|---|---|---|---|---|
| Cytokine | (μg/mL) | FIE | (μg/mL) | FIE | Predicted | served | EOB |
| IL-1β | 5.00 | 0.38 | 0.63 | 0.08 | 0.43 | 0.72 | 0.29 |
| | | | 1.25 | 0.07 | 0.42 | 0.82 | 0.40 |
| | | | 2.50 | 0.08 | 0.43 | 0.88 | 0.45 |
| | | | 5.00 | 0.17 | 0.48 | 0.91 | 0.43 |
| | | | 10.00 | 0.68 | 0.80 | 0.92 | 0.12 |
| | | | 20.00 | 0.81 | 0.88 | 0.98 | 0.10 |
| IL-6 | 5.00 | 0.08 | 0.63 | 0.12 | 0.19 | 0.26 | 0.07 |
| | | | 1.25 | 0.00 | 0.08 | 0.57 | 0.49 |
| | | | 2.50 | 0.10 | 0.17 | 0.43 | 0.26 |
| | | | 5.00 | 0.08 | 0.15 | 0.41 | 0.25 |
| | | | 10.00 | 0.00 | 0.08 | 0.81 | 0.73 |
| | | | 20.00 | 0.12 | 0.19 | 0.93 | 0.74 |
| TNF-α | 5.00 | 0.05 | 0.63 | 0.00 | 0.06 | 0.34 | 0.28 |
| | | | 1.25 | 0.08 | 0.13 | 0.46 | 0.33 |
| | | | 2.50 | 0.05 | 0.11 | 0.57 | 0.47 |
| | | | 5.00 | 0.02 | 0.07 | 0.63 | 0.56 |
| | | | 10.00 | 0.04 | 0.10 | 0.62 | 0.52 |
| | | | 20.00 | 0.30 | 0.34 | 0.90 | 0.57 |
| IL-1α | 5.00 | 0.40 | 0.63 | 0.03 | 0.41 | 0.66 | 0.25 |
| | | | 1.25 | 0.01 | 0.40 | 0.80 | 0.40 |
| | | | 2.50 | 0.12 | 0.47 | 0.85 | 0.38 |
| | | | 5.00 | 0.24 | 0.54 | 0.89 | 0.35 |
| | | | 10.00 | 0.50 | 0.70 | 0.88 | 0.18 |
| | | | 20.00 | 0.54 | 0.72 | 0.96 | 0.24 |
| MIP-1α | 5.00 | 0.12 | 2.50 | 0.00 | 0.12 | 0.26 | 0.14 |
| | | | 20.00 | 0.00 | 0.12 | 0.42 | 0.30 |

FIE—Fractional inhibitory effect;
EOB—Excess over Bliss

TABLE 20
Synergistic inhibition of IL-1β, IL-6, TNF-α, IL-1α, and MIP-
1α release by CBD and HCQ in human PBMCs (Donor 2)

| | CBD | | HCQ | | | Ob- | |
|---|---|---|---|---|---|---|---|
| Cytokine | (μg/mL) | FIE | (μg/mL) | FIE | Predicted | served | EOB |
| IL-1β | 5.00 | 0.13 | 0.63 | 0.06 | 0.19 | 0.34 | 0.15 |
| | | | 2.50 | 0.07 | 0.19 | 0.50 | 0.31 |
| IL-6 | 5.00 | 0.09 | 10.00 | 0.11 | 0.19 | 0.60 | 0.41 |
| | | | 20.00 | 0.47 | 0.52 | 0.83 | 0.31 |
| TNF-α | 5.00 | 0.07 | 5.00 | 0.02 | 0.09 | 0.18 | 0.09 |
| | | | 10.00 | 0.18 | 0.24 | 0.57 | 0.33 |
| IL-1α | 5.00 | 0.28 | 0.63 | 0.07 | 0.33 | 0.51 | 0.18 |
| | | | 1.25 | 0.11 | 0.36 | 0.61 | 0.24 |
| | | | 2.50 | 0.19 | 0.42 | 0.62 | 0.21 |
| | | | 5.00 | 0.49 | 0.63 | 0.74 | 0.11 |
| | | | 10.00 | 0.68 | 0.77 | 0.89 | 0.13 |
| | | | 20.00 | 0.63 | 0.73 | 0.92 | 0.19 |
| MIP-1α | 5.00 | 0.00 | 10.00 | 0.00 | 0.00 | 0.24 | 0.24 |
| | | | 20.00 | 0.00 | 0.00 | 0.62 | 0.62 |

FIE—Fractional inhibitory effect;
EOB—Excess over Bliss

TABLE 21
Synergistic inhibition of inflammatory cytokine release in vivo
following administration of 10 mg/kg CBD and 25 mg/kg HCQ

| | IL-6 | IL-12(p70) | IFN-γ | TNF-α |
|---|---|---|---|---|
| CBD 10 mg/kg | 0.14 | 0.00 | 0.00 | 0.00 |
| HCQ 25 mg/kg | 0.17 | 0.56 | 0.57 | 0.33 |
| Predicted CBD 10 mg/kg HCQ | 0.28 | 0.56 | 0.57 | 0.33 |
| 25 mg/kg | | | | |
| Observed CBD 10 mg/kg HCQ | 0.40 | 0.79 | 0.72 | 0.46 |
| 25 mg/kg | | | | |
| EOB | 0.12 | 0.24 | 0.15 | 0.14 |

EOB—Excess over Bliss

TABLE 22
Synergistic inhibition of inflammatory cytokine release in vivo
following administration of 10 mg/kg CBD and 100 mg/kg HCQ

| | | | IL- | | |
|---|---|---|---|---|---|
| | IL-1β | IL-6 | 12(p70) | IFN-γ | TNF-α |
| CBD 10 mg/kg | 0.34 | 0.14 | 0.00 | 0.00 | 0.00 |
| HCQ 100 mg/kg | 0.01 | 0.22 | 0.72 | 0.74 | 0.55 |
| Predicted CBD 10 mg/kg HCQ | 0.35 | 0.33 | 0.72 | 0.74 | 0.55 |
| 100 mg/kg | | | | | |
| CBD 10 mg/kg HCQ 100 mg/kg | 0.65 | 0.35 | 0.87 | 0.83 | 0.83 |
| EOB | 0.30 | 0.03 | 0.15 | 0.09 | 0.28 |

EOB—Excess over Bliss

TABLE 23
Synergistic inhibition of inflammatory cytokine release in vivo
following administration of 25 mg/kg CBD and 25 mg/kg HCQ

| | IL-6 |
|---|---|
| CBD 25 mg/kg | 0.03 |
| HCQ 25 mg/kg | 0.17 |
| Predicted CBD 25 mg/kg HCQ 25 | 0.19 |
| mg/kg | |
| CBD 25 mg/kg HCQ 25 mg/kg | 0.33 |
| EOB | 0.15 |

EOB—Excess over Bliss

TABLE 24
Synergistic inhibition of inflammatory cytokine release in vivo
following administration of 25 mg/kg CBD and 100 mg/kg HCQ

| | IL-6 |
|---|---|
| CBD 25 mg/kg | 0.03 |
| HCQ 100 mg/kg | 0.22 |
| Predicted CBD 25 mg/kg HCQ 25 | 0.24 |
| mg/kg | |
| CBD 25 mg/kg HCQ 100 mg/kg | 0.36 |
| EOB | 0.13 |

EOB—Excess over Bliss

TABLE 25
Synergistic inhibition of inflammatory cytokine release
in an in vivo model of pulmonary inflammation following
administration of 1 mg/kg CBD and 25 mg/kg HCQ

| | IL-1β | IL-6 | MCP-1 | TNF-α |
|---|---|---|---|---|
| CBD 1 mg/kg | 0.03 | 0.15 | 0.07 | 0.23 |
| HCQ 25 mg/kg | 0.20 | 0.08 | 0.09 | 0.14 |
| CBD 1 mg/kg HCQ 25 mg/kg | 0.34 | 0.40 | 0.38 | 0.39 |
| Predicted CBD 1 mg/kg HCQ 25 mg/kg | 0.22 | 0.21 | 0.15 | 0.34 |
| EOB | 0.12 | 0.19 | 0.23 | 0.05 |

EOB—Excess over Bliss

TABLE 26
Synergistic inhibition of inflammatory cytokine release
in an in vivo model of pulmonary inflammation following
administration of 10 mg/kg CBD and 25 mg/kg HCQ

| | CXCL-1 |
|---|---|
| CBD 10 mg/kg | 0.14 |
| HCQ 25 mg/kg | 0.35 |
| CBD 10 mg/kg HCQ 25 mg/kg | 0.51 |
| Predicted CBD 10 mg/kg HCQ 25 mg/kg | 0.44 |
| EOB | 0.06 |

EOB—Excess over Bliss

TABLE 27
Relative reduction in white blood cell counts
in an in vivo model of pulmonary inflammation

| | WBC | Neutrophil |
|---|---|---|
| CBD 10 mg/kg | 0.27 | 0.25 |
| HCQ 2.5 mg/kg | 0.11 | 0.10 |
| CBD 10 mg/kg HCQ 2.5 mg/kg | 0.32 | 0.30 |

TABLE 28
Synergistic activity of 1 mg/kg CBD and 2.5
mg/kg HCQ in an in vivo model of colitis

| | | Stool consistency | Macroscopic |
|---|---|---|---|
| | MPO | score | damage score |
| CBD 1 mg/kg | 0.34 | 0.17 | 0.10 |
| HCQ 2.5 mg/kg | 0.00 | 0.00 | 0.05 |
| Predicted CBD + HCQ | 0.34 | 0.17 | 0.15 |
| Observed CBD + HCQ | 0.46 | 0.33 | 0.17 |
| EOB | 0.12 | 0.17 | 0.02 |

EOB—Excess over Bliss

TABLE 29
Relative reduction in Total Colitis Index and
Colon to Body Weight Ratio of 1 mg/kg CBD and
2.5 mg/kg HCQ in an in vivo model of colitis

| | Colon to body weight ratio | Colitis index |
|---|---|---|
| CBD 1 mg/kg | 0.373 | 0.25 |
| HCQ 2.5 mg/kg | 0.287 | 0.27 |
| CBD + HCQ | 0.393 | 0.46 |

TABLE 30
Relative reduction in pulmonary lesion score of 1 mg/kg CBD and
2.5 mg/kg HCQ in an in vivo model of pulmonary inflammation

| | Relative reduction in lesion score |
|---|---|
| CBD 1 mg/kg | 0.18 |
| HCQ 2.5 mg/kg | 0.21 |
| CBD 1 mg/kg + HCQ 2.5 mg/kg | 0.31 |

TABLE 31
Relative reduction in pulmonary lesion score of 10 mg/kg CBD and
25 mg/kg HCQ in an in vivo model of pulmonary inflammation

| | Relative reduction in lesion score |
|---|---|
| CBD 10 mg/kg | 0.29 |
| HCQ 25 mg/kg | 0.37 |
| CBD 10 mg/kg HCQ 25 mg/kg | 0.46 |

TABLE 32
Disease severity score matrix for in vivo model of arthritis

| Score | Condition |
|---|---|
| 0 | Normal |
| 1 | Mild, but definite redness and swelling of the ankle or wrist, or |
| | apparent redness and swelling limited to individual digits, |
| | regardless of the number of affected digits |
| 2 | Moderate redness and swelling of ankle of wrist |
| 3 | Severe redness and swelling of the entire paw including digits |
| 4 | Maximally inflamed limb with involvement of multiple joints |

TABLE 33
Cartilage and bone destruction by pannus formation
scoring matrix in vivo model of arthritis

| Score | Condition |
|---|---|
| 0 | no change |
| 1 | Mild change- pannus formation within cartilage |
| 2 | Moderate change- pannus invasion into cartilage/subchondral bone |
| 3 | Severe change- pannus invasion into the subchondral bone |

TABLE 34
Mononuclear cell infiltration scoring
matrix for in vivo model of arthritis

| Score | Condition |
|---|---|
| 0 | no infiltration |
| 1 | Mild infiltration |
| 2 | Moderate infiltration |
| 3 | Severe infiltration |

TABLE 35
Synergistic activity of 1 mg/kg CBD and 2.5
mg/kg HCQ in an in vivo model of arthritis

| | Clinical | | | |
|---|---|---|---|---|
| | score Day | Paw volume Day | Pannus | Total histology |
| | 24 | 24 | score | score |
| CBD | 0.21 | 0.04 | 0.07 | 0.11 |
| HCQ | 0.18 | 0.04 | 0.07 | 0.15 |
| Pred CBD + | 0.35 | 0.07 | 0.13 | 0.24 |
| HCQ | | | | |
| Obs CBD + | 0.40 | 0.34 | 0.43 | 0.28 |
| HCQ | | | | |
| EOB | 0.05 | 0.26 | 0.30 | 0.03 |

EOB—Excess over Bliss

TABLE 36
Relative reduction in serum cytokine levels of 1 mg/kg
CBD and 2.5 mg/kg HCQ in an in vivo model of arthritis

| | IL-1β | IL-6 |
|---|---|---|
| CBD | 0.31 | 0.50 |
| HCQ | 0.37 | 0.51 |
| CBD + HCQ | 0.25 | 0.36 |

TABLE 37
Comparison of high dose HCQ with low dose HCQ
in combination with 1 mg/kg CBD in reducing disease
severity in an in vivo model of arthritis

| | | CBD 1 mg/kg + |
|---|---|---|
| | HCQ 25 mg/kg | HCQ 2.5 mg/kg |
| Clinical score Day 24 | 0.21 | 0.40 |
| Clinical score Day 30 | 0.26 | 0.31 |
| Paw volume Day 24 | 0.10 | 0.34 |
| Paw volume Day 30 | 0.20 | 0.23 |
| Pannus score | 0.28 | 0.43 |
| Mononuclear cell infiltration | 0.24 | 0.15 |
| Total histology score | 0.26 | 0.28 |
| IL-1B | 0.25 | 0.39 |
| IL-6 | 0.36 | 0.72 |

1. A method for the treatment or prevention of an inflammatory condition, the method comprising administering to a subject in need thereof an effective amount of cannabidiol (CBD) or a pharmaceutically acceptable salt thereof, and an effective amount of hydroxychloroquine or a pharmaceutically acceptable salt thereof.

2. The method of claim 1, wherein the inflammatory condition is selected from the group consisting of an inflammatory respiratory condition, inflammatory bowel disease and arthritis.

3. The method of claim 2, wherein the inflammatory respiratory condition is selected from the group consisting of chronic obstructive pulmonary disease (COPD), asthma, bronchitis, cystic fibrosis (CF) and acute respiratory distress syndrome (ARDS).

4. The method of claim 3, wherein the ARDS is sepsis-associated ARDS.

5. The method of claim 4, wherein the sepsis-associated ARDS is caused by a bacterial, fungal, or viral infection.

6. The method of claim 1, wherein the hydroxychloroquine is administered at a dose of from about 1 mg to about 1000 mg.

7. The method of claim 1, wherein the CBD is a synthetic compound.

8. The method of claim 7, wherein the CBD is administered at a dose of from about 1 mg to about 1500 mg.

9. The method of claim 1, wherein the CBD and hydroxychloroquine are administered twice per day.

10. The method of claim 1, wherein the CBD and hydroxychloroquine are orally administered.

11. The method of claim 1, wherein the CBD and hydroxychloroquine are sequentially administered.

12. The method of claim 1, wherein the CBD and hydroxychloroquine are simultaneously administered.

13. The method of claim 1, wherein the CBD and hydroxychloroquine are administered to the subject for a period of at least one week.

14. The method of claim 1, wherein the CBD and hydroxychloroquine are administered to the subject for a period of between 1 to 10 weeks.

15. (canceled)

16. A composition comprising CBD or a pharmaceutically acceptable salt thereof, and hydroxychloroquine or a pharmaceutically acceptable salt thereof.

17. The composition of claim 16, wherein the composition further comprises one or more pharmaceutically acceptable carriers, diluents and excipients.

18. The composition of claim 16, wherein the composition comprises about 1 mg to about 1000 mg hydroxychloroquine per dose.

19. The composition of claim 16, wherein the composition comprises about 1 mg to about 1500 mg CBD per dose.

20. The composition of claim 16, wherein said composition is used to treat prevent an inflammatory condition.

21. The composition of claim 20, wherein the inflammatory condition is selected from the group consisting of an inflammatory respiratory condition, inflammatory bowel disease and arthritis.

22. The composition of claim 21, wherein the inflammatory respiratory condition is selected from selected from the group consisting of chronic obstructive pulmonary disease (COPD), asthma, cystic fibrosis (CF) and acute respiratory distress syndrome (ARDS).

23. The composition of claim 22, wherein the inflammatory respiratory condition is ARDS.

24. The composition of claim 23, wherein the ARDS is sepsis-associated ARDS.

25. The composition of claim 24, wherein the sepsis-associated ARDS is caused by a bacterial, fungal, or viral infection.

26. A kit comprising CBD or a pharmaceutically acceptable salt thereof, and hydroxychloroquine or a pharmaceutically acceptable salt thereof.

27. The kit of claim 26, wherein the CBD and hydroxychloroquine are in separate unit dosage forms for a loading dose and a maintenance dose.

28. A method of modulating an immune response comprising administering to a subject in need thereof an effective amount of CBD or a pharmaceutically acceptable salt thereof, and hydroxychloroquine or a pharmaceutically acceptable salt thereof.
